(19) United States
(12) Patent Application Publication
(10) Pub. No.: US 2017/0275330 A1
(43) Pub. Date: Sep. 28, 2017

(54) POLYMORPHIC FORMS OF AN OXYSTEROL AND METHODS OF MAKING THEM

(71) Applicant: Warsaw Orthopedic, Inc., Warsaw, IN (US)

(72) Inventors: Roger E. Harrington, Collierville, TN (US); Jerbrena C. Jacobs, Hernando, MS (US)

(21) Appl. No.: 15/082,695

(22) Filed: Mar. 28, 2016

(51) Int. Cl. C07J 9/00

(52) U.S. Cl. CPC C07J 9/00

(57) ABSTRACT

Compositions and methods for preparing an OXY133 polymorph are provided. This compositions and methods include subjecting a slurry of OXY133 to conditions sufficient to convert OXY133 to the OXY133 polymorph Form A, polymorph Form B, polymorph Form C, polymorph Form D, polymorph Form E, polymorph Form F, polymorph Form G, polymorph Form H, polymorph Form I or a mixture thereof. A polymorph of OXY133 is also provided and that polymorph can be polymorph Form A, polymorph Form B, polymorph Form C, polymorph Form D, polymorph Form E, polymorph Form F, polymorph Form G, polymorph Form H, polymorph Form I or a mixture thereof. Pharmaceutical compositions including OXY133 polymorphs are also provided.

### BACKGROUND

Different biological substances are commonly employed to promote bone growth in medical applications including fracture healing and surgical management of bone disorders including spinal disorders. Spine fusion is often performed by orthopedic surgeons and neurosurgeons alike to address degenerative disc disease and arthritis affecting the lumbar and cervical spine. Historically, autogenous bone grafting, commonly taken from the iliac crest of the patient, has been used to augment fusion between vertebral levels.

One protein that is osteogenic and commonly used to promote spine fusion is recombinant human bone morphogenetic protein-2 (rhBMP-2). Its use has been approved by the US Food and Drug Administration (FDA) for single-level anterior lumbar interbody fusion. The use of rhBMP-2 has increased significantly since this time and indications for its use have expanded to include posterior lumbar spinal fusion as well as cervical spine fusion.

Oxysterols form a large family of oxygenated derivatives of cholesterol that are present in the circulation, and in human and animal tissues. Oxysterols have been found to be present in atherosclerotic lesions and play a role in various physiologic processes, such as cellular differentiation, inflammation, apoptosis, and steroid production. Some naturally occurring oxysterols have robust osteogenic properties and can be used to grow bone. The most potent osteogenic naturally occurring oxysterol, 20(S)-hydroxycholesterol, is both osteogenic and anti-adipogenic when applied to multipotent mesenchymal cells capable of differentiating into osteoblasts and adipocytes.

One such oxysterol is OXY133 or (3S,5S,6S,8R,9S,10R,13S,14S,17S) 17-((S)-2-hydroxyoctan-2-yl)-10, 13-dimethylhexadecahydro-1H-cyclopenta[a]phenanthrene-3,6-diol, which exhibits the following structures:

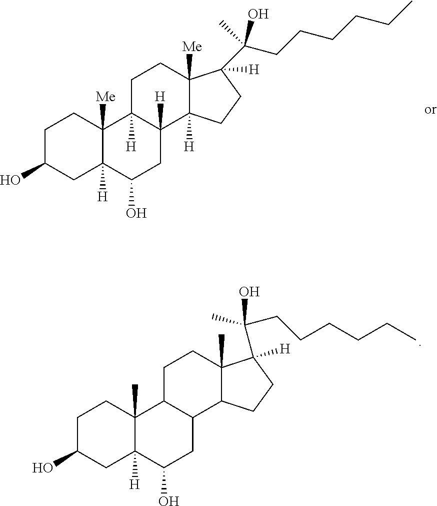

There is a need to develop different polymorphic forms of OXY133. There is also a need for providing a robust, reproducible and scalable process for the production of OXY133 monohydrate.

### SUMMARY

In some embodiments, compositions and methods for preparing an OXY133 polymorph are provided. These compositions and methods include subjecting a slurry of OXY133 to conditions sufficient to convert OXY133 to an OXY133 polymorph which comprises, consists essentially of or consists of polymorph Form A, polymorph Form B, polymorph Form C, polymorph Form D, polymorph Form E, polymorph Form F, polymorph Form G, polymorph Form H, polymorph Form I or a mixture thereof. In various aspects, the conditions comprise dissolving a slurry of OXY133 in a solvent and precipitating the OXY133 polymorph by adding an anti-solvent at a temperature sufficient to precipitate the OXY133 polymorph. OXY133 useful for preparing the polymorphs described in this disclosure comprises at least one of (i) anhydrous OXY133 or OXY133 polymorph Form B; (ii) an OXY133 polymorph other than polymorph Form B; (iii) a hydrate of OXY133; or (iv) a solvate of OXY133.

In other embodiments, the conditions to convert OXY133 to an OXY133 polymorph comprise mixing OXY133 with: (i) an isopropanol solvent, and a water anti-solvent in a ratio from about 1:1 volume by volume (v/v) to about 1:2 v/v at a temperature from about 0° C. to about 20° C. to obtain OXY133 polymorph Form A or OXY133 monohydrate; (ii) a tetrahydrofuran solvent, and a water anti-solvent in a ratio of about 1:2 v/v at a temperature from about 10° C. to about 35° C. to obtain OXY133 polymorph Form A or OXY133 monohydrate; (iii) a tetrahydrofuran/acetone solvent, and a water anti-solvent at a temperature of about 35° C. to obtain OXY133 polymorph Form A or OXY133 monohydrate; (iv) an acetone solvent, and a water anti-solvent in a ratio of about 1:1 v/v at a temperature of about 15° C. to about 25° C. to obtain OXY133 polymorph Form A or OXY133 monohydrate; (v) an acetone solvent, and a water anti-solvent in a ratio of about 1:1 v/v at a temperature of about 30° C. to about 60° C. to obtain OXY133 polymorph Form C; (vi) a methanol solvent, and a water anti-solvent in a ratio of about 1:1 v/v at a temperature of about 20° C. to about 70° C. to obtain OXY133 polymorph Form D; (vii) water at a temperature from about 20° C. to about 70° C. to obtain OXY133 polymorph Form E; (viii) an acetone solvent, and a water anti-solvent at a temperature from about 5° C. to about 15° C. to obtain OXY133 polymorph Form F; (ix) an isopropanol solvent, and a water anti-solvent in a ratio of about 1:2 v/v at a temperature of about 40° C. to obtain OXY133 polymorph Form G; (x) an isopropanol solvent, and a water anti-solvent in a ratio of about 1:2 at a temperature of about −10° C. to obtain OXY133 polymorph Form H; (xi) a methanol/acetone solvent, and a water anti-solvent at temperature of about 20° C. to obtain OXY133 polymorph Form I; or (xii) acetone recrystallization at about 20° C. to obtain OXY133 polymorph Form I.

In other embodiments, OXY133 is prepared by reacting a diol having the formula:

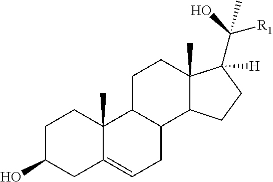

with borane, hydrogen peroxide and tetrahydrofuran to form an oxysterol or a pharmaceutically acceptable salt, hydrate or solvate thereof having the formula:

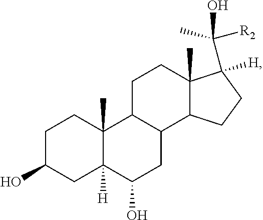

wherein R₁ and R₂ comprise a hexyl group and the diol comprises (3S,8S,9S,10R,13R,14S,17R)-10,13-dimethyl-17-[(S)-2-hydroxyoctan-yl]-2,3,4,7,8,9,11,12,14,15,16,17-dodecahydro-1H-cyclopenta[a]phenanthren-3-ol (OXY133).

Other aspects of this disclosure are directed to providing a method for preparing OXY133 monohydrate or OXY133 polymorph Form A, the method comprises, consists essentially of, or consists of, slurrying OXY133 in a solvent system under conditions sufficient to convert OXY133 to an OXY133 monohydrate or polymorph Form A. In some aspects, the slurrying step comprises dissolving OXY133 in a solvent and precipitating the OXY133 monohydrate by adding an anti-solvent. In various embodiments, a solvent useful for dissolving OXY133 comprises, consists essentially of, or consists of, isopropanol, tetrahydrofuran, tetrahydrofuran/acetone or acetone and the anti-solvent comprises, consists essentially of, or consists of water. In some embodiments, the slurrying step occurs at a stirring temperature from about from about 0° C. to about 20° C. In other embodiments, OXY133 polymorph Form A is formed when the solvent is isopropanol, the anti-solvent is water in a ratio of 1:2 v/v at a temperature from about 0° C. to about 20° C.

In yet other embodiments, OXY133 polymorph Form A can be obtained when the solvent for dissolving the OXY133 slurry is tetrahydrofuran, the anti-solvent is water in a ratio of 1:2 v/v at a temperature from about 10° C. to about 35° C. In some aspects, the water content of OXY133 monohydrate comprises, consists essentially of, or consists of, a range from about 3.25%, 3.30%, 3.35%, 3.40%, 3.45%, 3.50%, 3.55%, 3.60%, 3.65%, 3.70%, 3.75%, 3.80%, 3.85%, 3.90%, 3.95%, 4.00%, 4.05% to about 4.1% by weight. In other aspects, OXY133 monohydrate obtained by the methods of this disclosure has a yield of from about 85% to about 94% by weight. In yet other aspects, OXY133 monohydrate or OXY133 polymorph Form A obtained by the methods of this disclosure includes drying at about 20° C., which can be accomplished in a vacuum or a freeze dryer.

In other embodiments, this disclosure provides a method for isolating OXY133 monohydrate, the method comprising heating a mixture of anhydrous OXY133 with isopropanol at a temperature from about 25° C. to about 35° C., cooling the mixture to about 5° C., and precipitating OXY133 monohydrate from the cooled mixture by adding water to the mixture at a ratio of isopropanol to water of 1:2 v/v. In some aspects, the OXY133 monohydrate is dried at a temperature of about 20° C. The yield of OXY133 monohydrate obtained by methods described in this disclosure are from about 85% to about 94% by weight.

In some embodiments, this disclosure also provides an OXY133 polymorph which comprises, consists essentially of, or consists of, polymorph Form A, polymorph Form B, polymorph Form C, polymorph Form D, polymorph Form E, polymorph Form F, polymorph Form G, polymorph Form H, polymorph Form I or a mixture thereof. In an embodiment, the OXY133 polymorph is OXY133 polymorph Form A or OXY133 monohydrate. In another embodiment, the OXY133 polymorph Form A is from about 85%, 86%, 87%, 88%, 89%, 90′%, 91%, 92%, 93% to about 94% by weight of OXY133.

Other aspects of this disclosure provide a pharmaceutical composition which include an OXY133 polymorph selected from polymorph Form A, polymorph Form B, polymorph Form C, polymorph Form D, polymorph Form E, polymorph Form F, polymorph Form G, polymorph Form H, polymorph Form I or a mixture thereof and pharmaceutically acceptable excipients. In some embodiments, the pharmaceutical composition includes OXY133 polymorph Form A or OXY133 monohydrate and pharmaceutically acceptable excipients.

In various embodiments, the OXY133 polymorph included in the pharmaceutical composition comprises (i) Form A that produces an X-ray powder diffraction pattern (XRPD) comprising one or more of the following reflections: 16.4, 17.91 and 20.94±0.2 degree 2θ (ii) Form B that produces an X-ray powder diffraction pattern comprising one or more of the following reflections: 13.3, 16.1, and 18.82±0.2 degree 2θ; or a mixture thereof. In other embodiments, the pharmaceutical composition of this disclosure includes an OXY133 polymorph, wherein the XRPD of OXY133 polymorph Form A further comprises one or more of the following reflections: 6.1, 12.3, and 18.6±0.2 degree 2θ; the XRPD of OXY133 polymorph Form B further comprises one or more of the following reflections: 5.9, 11.9, and 17.96±0.2 degree 2θ; or a mixture thereof.

In some embodiments, compositions and methods for preparing an OXY133 polymorph are provided. These compositions and methods include subjecting a slurry of OXY133 to conditions sufficient to convert OXY133 to OXY133 monohydrate or polymorph Form A. In some embodiments, the OXY133 monohydrate or OXY133 polymorph Form A produces an X-ray powder diffraction (XRPD) pattern comprising, consisting essentially of or consisting of one or more of the following reflections: 16.4, 17.91 and 20.94±0.2 degree 2θ. In other embodiments, the XRPD of OXY133 polymorph Form A or OXY133 monohydrate further comprises, consists essentially of or consists of one or more of the following reflections: 6.1, 12.3, 18.6±0.2 degree 2θ. In yet other embodiments, the methods of this disclosure provides OXY133 polymorph Form B which produces an X-ray powder diffraction (XRPD) pattern comprising, consisting essentially of or consisting of one or more of the following reflections: 13.3, 16.1 and 18.82±0.2 degree 2θ. In other embodiments, the XRPD of OXY133 polymorph Form B further comprises, consists essentially of or consists of one or more of the following reflections: 5.9, 11.9 and 17.96±0.2 degree 2θ.

In various aspects, the conditions comprise dissolving a slurry of OXY133 in a solvent and precipitating the OXY133 polymorph by adding an anti-solvent at a temperature sufficient to precipitate the OXY133 polymorph. OXY133 useful for preparing the polymorphs described in this disclosure comprises at least one of (i) anhydrous OXY133 or OXY133 polymorph Form B; (ii) an OXY133 polymorph other than polymorph Form B; (iii) a hydrate of OXY133; or (iv) a solvate of OXY133.

In other embodiments, the conditions to convert OXY133 to an OXY133 polymorph comprise mixing OXY133 with: (i) an isopropanol solvent, and a water anti-solvent in a ratio from about 1:1 volume by volume (v/v) to about 1:2 v/v at a temperature from about 0° C. to about 20° C. to obtain OXY133 polymorph Form A or OXY133 monohydrate; (ii) a tetrahydrofuran solvent, and a water anti-solvent in a ratio of about 1:2 v/v at a temperature from about 10° C. to about 35° C. to obtain OXY133 polymorph Form A or OXY133 monohydrate; (iii) a tetrahydrofuran/acetone solvent, and a water anti-solvent at a temperature of about 35° C. to obtain OXY133 polymorph Form A or OXY133 monohydrate; or (iv) an acetone solvent, and a water anti-solvent in a ratio of about 1:1 v/v at a temperature of about 15° C. to about 25° C. to obtain OXY133 polymorph Form A or OXY133 monohydrate.

In other embodiments, this disclosure provides a method for isolating OXY133 monohydrate, the method comprising heating a mixture of anhydrous OXY133 with isopropanol at a temperature from about 25° C. to about 35° C., cooling the mixture to about 5° C., and precipitating OXY133 monohydrate from the cooled mixture by adding water to the mixture at a ratio of isopropanol to water of 1:2 v/v. In some aspects, the OXY133 monohydrate is dried at a temperature of about 20° C. The yield of OXY133 monohydrate obtained by methods described in this disclosure are from about 85% to about 94% by weight.

Other aspects of this disclosure provide a pharmaceutical composition which includes OXY133 polymorph Form A or OXY133 monohydrate. In some embodiments, the pharmaceutical composition includes OXY133 polymorph Form A or OXY133 monohydrate and pharmaceutically acceptable excipients.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

In part, other aspects, features, benefits and advantages of the embodiments will be apparent with regard to the following description, appended claims and accompanying drawings where:

FIG. 1 illustrates a step-wise reaction for synthesizing OXY133 with starting reactants comprising pregnenolone acetate, as shown in one embodiment of this disclosure. The pregnenolone is reacted with an organometallic compound to produce a sterol or diol having two hydroxyl groups. The sterol or diol is then reacted with borane and hydrogen peroxide and purified to produce OXY133;

FIGS. 2A, 2B, 2C, 2D, and 2E are XRPDs of OXY133 polymorph Forms A and B:

FIG. 3 is a graphic illustration of XRPDs of OXY133 polymorph Forms A, B, C, D, E, F, G, H and I;

FIG. 4 is an XRPD of OXY133 polymorph Form A and unknown;

FIG. 5 is an XRPD of OXY133 polymorph Form B;

FIGS. 6A, 6B, 6C, 6D, 6E, 6F, 6G, 6H, 6I, 6J, 6K, 6L, 6M, 6N, 60, 6P, 6Q, 6R, 6S, 6T, 6U, 6V, 6W and 6X are XRPDs of OXY133 polymorph Form A;

FIGS. 7A, 7B, 7C, 7D, 7E, 7F, 7G, 7H, 7I, 7J, 7K, 7L, 7M, 7N, 70, and 7P are XRPDs of OXY133 polymorph Form C;

FIGS. 8A and 8B are XRPDs of OXY133 polymorph Form D;

FIGS. 9A and 9B are XRPDs of OXY133 polymorph Form E;

FIGS. 10A, 10B, 10C are XRPDs of OXY133 polymorph Form F;

FIG. 11 is an XRPD of OXY133 polymorph Form G;

FIG. 12 is an XRPD of OXY133 polymorph Form H;

FIGS. 13A and 13B are XRPDs of OXY133 polymorph Form I;

FIG. 14 is an HPLC/CAD single injection report of OXY133 polymorph Form B;

FIGS. 15A, 15B, 15C, 15D, 15E, 15F, 15G, 15H and 15I are HPLC/CAD single injection reports of OXY133 polymorph Form A;

FIG. 16 is a HPLC/CAD single injection reports of OXY133 polymorph Form I;

FIG. 17 is a HPLC/CAD single injection report of OXY133 polymorph Form H;

FIG. 18 is a HPLC/CAD single injection report of OXY133 sample 2891-12-4;

FIG. 19 is a HPLC/CAD single injection report of OXY133 sample 2891-15-1;

FIG. 20 is a DSC-TGA thermogram of OXY133 polymorph Form B;

FIGS. 21A, 21B, 21C, 21D, 21E, 21F, 21G, 21H, and 21I are DSC-TGA thermograms of OXY133 polymorph Form A;

FIG. 22 is a DSC-TGA thermogram of OXY133 polymorph Form C;

FIG. 23 is a DSC-TGA thermogram of OXY133 polymorph Form G;

FIG. 24 is a DSC-TGA thermogram of OXY133 polymorph Form H; and

FIG. 25 is a DSC-TGA thermogram of OXY133 polymorph Form A and unknown.

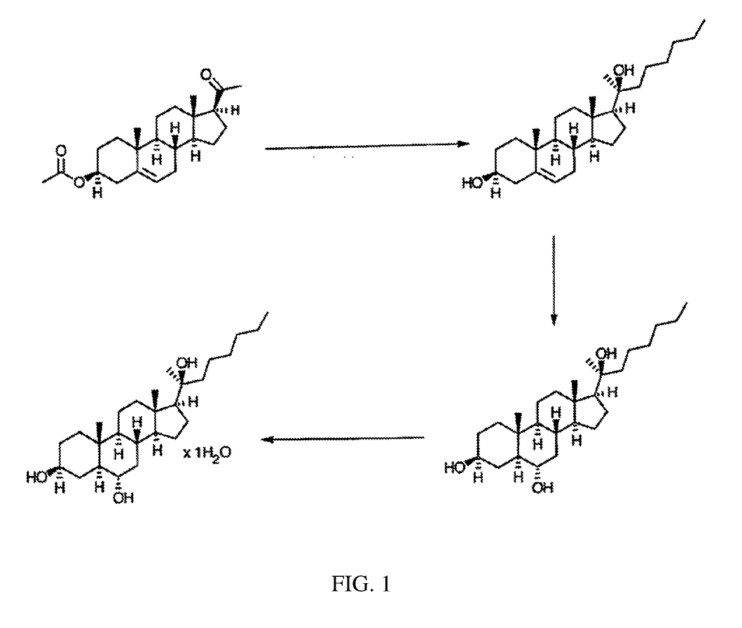

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the present application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub ranges subsumed therein. For example, a range of “1 to 10” includes any and all sub ranges between (and including) the minimum value of 1 and the maximum value of 10, that is, any and all sub ranges having a minimum value of equal to or greater than 1 and a maximum value of equal to or less than 10, e.g., 5.5 to 10.

### Definitions

It is noted that, as used in this specification and the appended claims, the singular forms “a,” “an,” and “the,” include plural referents unless expressly and unequivocally limited to one referent. Thus, for example, reference to “an alkanolamine” includes one, two, three or more alkanolamines.

The term “anti-solvent,” as used herein, refers to a solvent in which a compound is substantially insoluble. An anti-solvent useful in this disclosure includes, but is not limited to, water.

The term “crystalline,” as used herein, means having a regularly repeating arrangement of molecules or external face planes.

The term “crystalline composition,” as used in herein, refers to a solid chemical compound or mixture of compounds that provides a characteristic pattern of peaks when analyzed by x-ray powder diffraction, this includes, but is not limited to, polymorphs, solvates, hydrates, co-crystals, or desolvated solvates.

The term “bioactive agent” as used herein is generally meant to refer to any substance that alters the physiology of a patient. The term “bioactive agent” may be used interchangeably herein with the terms “therapeutic agent,” “therapeutically effective amount,” and “active pharmaceutical ingredient”, “API” or “drug”. OXY133 is an example of a bioactive agent. Bioactive or pharmaceutical compositions are sometimes referred to herein as “pharmaceutical compositions” or “bioactive compositions” of the current disclosure. Sometimes the phrase “administration of OXY133” is used herein in the context of administration of this compound to a subject (e.g., contacting the subject with the compound, injecting the compound, administering the compound in a drug depot, etc.). It is to be understood that the compound for such a use can generally be in the form of a pharmaceutical composition or bioactive composition comprising the OXY133. It will be understood that unless otherwise specified a “drug” formulation may include more than one therapeutic agent, wherein exemplary combinations of therapeutic agents include a combination of two or more drugs. The term “drug” is also meant to refer to the “API” whether it is in a crude mixture or purified or isolated.

The term “biodegradable” includes compounds or components that will degrade over time by the action of enzymes, by hydrolytic action and/or by other similar mechanisms in the human body. In various embodiments, “biodegradable” includes that components can break down or degrade within the body to non-toxic components as cells (e.g., bone cells) infiltrate the components and allow repair of the defect. By “bioerodible” it is meant that the compounds or components will erode or degrade over time due, at least in part, to contact with substances found in the surrounding tissue, fluids or by cellular action. By “bioabsorbable” it is meant that the compounds or components will be broken down and absorbed within the human body, for example, by a cell or tissue. “Biocompatible” means that the compounds or components will not cause substantial tissue irritation or necrosis at the target tissue site and/or will not be carcinogenic.

The term “alkyl” as used herein, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkenyl” and/or “alkynyl” is used, as defined below. In some embodiments, the alkyl groups are (C1-C40) alkyl. In some embodiments, the alkyl groups are (C1-C6) alkyl.

The term “alkanyl” as used herein refers to a saturated branched, straight-chain or cyclic alkyl radical derived by the removal of one hydrogen atom from a single carbon atom of a parent alkane. Typical alkanyl groups include, but are not limited to, methanyl; ethenyl; propanyls such as propan-1-yl, propan-2-yl(isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. In some embodiments, the alkanyl groups are (C1-C40) alkanyl. In some embodiments, the alkanyl groups are (C1-C6) alkanyl.

The term “alkenyl” as used herein refers to an unsaturated branched, straight-chain or cyclic alkyl radical having at least one carbon-carbon double bond derived by the removal of one hydrogen atom from a single carbon atom of a parent alkene. The radical may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In some embodiments, the alkenyl group is (C2-C40) alkenyl. In some embodiments, the alkenyl group is (C2-C6) alkenyl.

The term “alkynyl” as used herein refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-3-yn-1-yl, etc.; and the like. In some embodiments, the alkynyl group is (C2-C40) alkynyl. In some embodiments, the alkynyl group is (C2-C6) alkynyl.

The term “alkyldiyl” as used herein refers to a saturated or unsaturated, branched, straight-chain or cyclic divalent hydrocarbon radical derived by the removal of one hydrogen atom from each of two different carbon atoms of a parent alkane, alkene or alkyne, or by the removal of two hydrogen atoms from a single carbon atom of a parent alkane, alkene or alkyne.

The two monovalent radical centers or each valency of the divalent radical center can form bonds with the same or different atoms. Typical alkyldiyls include, but are not limited to methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-, 1-diyl, prop-1-en-1,2-diyl, prop-2-en-1,2-diyl, prop-1-en-1,3-diyl cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,3-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. In some embodiments, the alkyldiyl group is (C1-C40) alkyldiyl. In some embodiments, the alkyldiyl group is (C1-C6) alkyldiyl. Also contemplated are saturated acyclic alkanyldiyl radicals in which the radical centers are at the terminal carbons, e.g., methandiyl (methano); ethan-1,2-diyl(ethano); propan-1,3-diyl(propano); butan-1,4-diyl(butano); and the like (also referred to as alkylenos, defined infra).

The term “alkyleno” as used herein refers to a straight-chain alkyldiyl radical having two terminal monovalent radical centers derived by the removal of one hydrogen atom from each of the two terminal carbon atoms of straight-chain parent alkane, alkene or alkyne. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, but[1,3]diyno, etc.; and the like. Where specific levels of saturation are intended, the nomenclature alkano, alkeno and/or alkyno is used. In some embodiments, the alkyleno group is (C1-C40) alkyleno. In some embodiments, the alkyleno group is (C1-C6) alkyleno.

The terms “heteroalkyl,” “heteroalkanyl,” “heteroalkenyl,” “heteroalkanyl,” “heteroalkyldiyl” and “heteroalkyleno” as used herein refer to alkyl, alkanyl, alkenyl, alkynyl, alkyldiyl and alkyleno radicals, respectively, in which one or more of the carbon atoms are each independently replaced with the same or different heteroatomic groups. Typical heteroatomic groups which can be included in these radicals include, but are not limited to, —O—, —S—, —O—O—, —S—S—, —O—S—, —NR′, ═N—N═, —N═N—, —N(O)N—, —N═N—NR′—, —PH—, —P(O)₂—, —O—P(O)₂—, —SH₂—, —S(O)₂—, or the like, where each R′ is independently hydrogen, alkyl, alkanyl, alkenyl, alkynyl, aryl, arylaryl, arylalkyl, heteroaryl, heteroarylalkyl or heteroaryl-heteroaryl as defined herein.

The term “aryl” as used herein refers to a monovalent aromatic hydrocarbon radical derived by the removal of one hydrogen atom from a single carbon atom of a parent aromatic ring system. Typical aryl groups include, but are not limited to, radicals derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In some embodiments, the aryl group is (C5-C14) aryl or a (C5-C10) aryl. Some preferred aryls are phenyl and naphthyl.

The term “aryldiyl” as used herein refers to a divalent aromatic hydrocarbon radical derived by the removal of one hydrogen atom from each of two different carbon atoms of a parent aromatic ring system or by the removal of two hydrogen atoms from a single carbon atom of a parent aromatic ring system. The two monovalent radical centers or each valency of the divalent center can form bonds with the same or different atom(s). Typical aryldiyl groups include, but are not limited to, divalent radicals derived from aceanthrylene, acenaphthylene, acephenanthrylene, anthracene, azulene, benzene, chrysene, coronene, fluoranthene, fluorine, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In some embodiments, the aryldiyl group is (C5-C14) aryldiyl or (C5-C10) aryldiyl. For example, some preferred aryldiyl groups are divalent radicals derived from benzene and naphthalene, especially phena-1,4-diyl, naphtha-2,6-diyl and naphtha-2,7-diyl.

The term “arydeno” as used herein refers to a divalent bridge radical having two adjacent monovalent radical centers derived by the removal of one hydrogen atom from each of two adjacent carbon atoms of a parent aromatic ring system. Attaching an aryleno bridge radical, e.g. benzeno, to a parent aromatic ring system, e.g. benzene, results in a fused aromatic ring system, e.g. naphthalene. The bridge is assumed to have the maximum number of non-cumulative double bonds consistent with its attachment to the resultant fused ring system. In order to avoid double-counting carbon atoms, when an aryleno substituent is formed by taking together two adjacent substituents on a structure that includes alternative substituents, the carbon atoms of the aryleno bridge replace the bridging carbon atoms of the structure. As an example, consider the following structure:

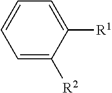

wherein R¹, when taken alone is hydrogen, or when taken together with R² is (C5-C14) aryleno; and R², when taken alone is hydrogen, or when taken together with R¹ is (C5-C14) aryleno.

When R¹ and R² are each hydrogen, the resultant compound is benzene. When R¹ taken together with R² is C6 aryleno (benzeno), the resultant compound is naphthalene. When R¹ taken together with R² is C10 aryleno (naphthaleno), the resultant compound is anthracene or phenanthrene. Typical aryleno groups include, but are not limited to, aceanthryleno, acenaphthyleno, acephenanthtyleno, anthraceno, azuleno, benzeno (benzo), chryseno, coroneno, fluorantheno, fluoreno, hexaceno, hexapheno, hexyleno, as-indaceno, s-indaceno, indeno, naphthalene (naphtho), octaceno, octapheno, octaleno, ovaleno, penta-2,4-dieno, pentaceno, pentaleno, pentapheno, peryleno, phenaleno, phenanthreno, piceno, pleiadeno, pyreno, pyranthreno, rubiceno, triphenyleno, trinaphthaleno, and the like. Where a specific connectivity is intended, the involved bridging carbon atoms (of the aryleno bridge) are denoted in brackets, e.g., [1,2]benzeno ([1,2]benzo), [1,2]naphthaleno, [2,3]naphthaleno, etc. Thus, in the above example, when R¹ taken together with R² is [2,3]naphthaleno, the resultant compound is anthracene. When R¹ taken together with R² is [1,2]naphthaleno, the resultant compound is phenanthrene. In a preferred embodiment, the aryleno group is (C5-C14), with (C5-C10) being even more preferred.

The term “arylaryl” as used herein refers to a monovalent hydrocarbon radical derived by the removal of one hydrogen atom from a single carbon atom of a ring system in which two or more identical or non-identical parent aromatic ring systems are joined directly together by a single bond, where the number of such direct ring junctions is one less than the number of parent aromatic ring systems involved. Typical arylaryl groups include, but are not limited to, biphenyl, triphenyl, phenyl-naphthyl, binaphthyl, biphenyl-naphthyl, and the like. When the number of carbon atoms comprising an arylaryl group is specified, the numbers refer to the carbon atoms comprising each parent aromatic ring. For example, (C1-C14) arylaryl is an arylaryl group in which each aromatic ring comprises from 5 to 14 carbons, e.g., biphenyl, triphenyl, binaphthyl, phenylnaphthyl, etc. In some instances, each parent aromatic ring system of an arylaryl group is independently a (C5-C14) aromatic or a (C1-C10) aromatic. Some preferred are arylaryl groups in which all of the parent aromatic ring systems are identical, e.g., biphenyl, triphenyl, binaphthyl, trinaphthyl, etc.

The term “biaryl” as used herein refers to an arylaryl radical having two identical parent aromatic systems joined directly together by a single bond. Typical biaryl groups include, but are not limited to, biphenyl, binaphthyl, bianthracyl, and the like. In some instances, the aromatic ring systems are (C5-C14) aromatic rings or (C5-C10) aromatic rings. One preferred biaryl group is biphenyl.

The term “arylalkyl” as used herein refers to an acyclic alkyl radical in which one of the hydrogen atoms bonded to a carbon atom, typically a terminal or spa carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In some embodiments, the arylalkyl group is (C6-C40) arylalkyl, e.g., the alkanyl, alkenyl or alkynyl moiety of the arylalkyl group is (C1-C26) and the aryl moiety is (C5-C14). In some preferred embodiments the arylalkyl group is (C6-C13), e.g., the alkanyl, alkenyl or alkynyl moiety of the arylalkyl group is (C1-C3) and the aryl moiety is (C5-C10).

The term “heteroaryl” as used herein refers to a monovalent heteroaromatic radical derived by the removal of one hydrogen atom from a single atom of a parent heteroaromatic ring system. Typical heteroaryl groups include, but are not limited to, radicals derived from acridine, arsindole, carbazole, f-carboline, chromane, chromene, cinnoline, furan, imidazole, indazole, indole, indoline, indolizine, isobenzofuran, isochromene, isoindole, isoindo line, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group is a 5-14 membered heteroaryl, with 5-10 membered heteroaryl being particularly preferred. Some preferred heteroaryl radicals are those derived from parent heteroaromatic ring systems in which any ring heteroatoms are nitrogens, such as imidazole, indole, indazole, isoindole, naphthyridine, pteridine, isoquinoline, phthalazine, purine, pyrazole, pyrazine, pyridazine, pyridine, pyrrole, quinazoline, quinoline, etc.

The term “heteroaryldiyl” refers to a divalent heteroaromatic radical derived by the removal of one hydrogen atom from each of two different atoms of a parent heteroaromatic ring system or by the removal of two hydrogen atoms from a single atom of a parent heteroaromatic ring system. The two monovalent radical centers or each valency of the single divalent center can form bonds with the same or different atom(s). Typical heteroaryldiyl groups include, but are not limited to, divalent radicals derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryldiyl group is 5-14 membered heteroaryldiyl or a 5-10 membered heteroaryldiyl. Some preferred heteroaryldiyl groups are divalent radicals derived from parent heteroaromatic ring systems in which any ring heteroatoms are nitrogens, such as imidazole, indole, indazole, isoindole, naphthyridine, pteridine, isoquinoline, phthalazine, purine, pyrazole, pyrazine, pyridazine, pyridine, pyrrole, quinazoline, quinoline, etc.

The term “heteroaryleno” as used herein refers to a divalent bridge radical having two adjacent monovalent radical centers derived by the removal of one hydrogen atom from each of two adjacent atoms of a parent heteroaromatic ring system. Attaching a heteroaryleno bridge radical, e.g. pyridino, to a parent aromatic ring system, e.g. benzene, results in a fused heteroaromatic ring system, e.g., quinoline. The bridge is assumed to have the maximum number of non-cumulative double bonds consistent with its attachment to the resultant fused ring system. In order to avoid double-counting ring atoms, when a heteroaryleno substituent is formed by taking together two adjacent substituents on a structure that includes alternative substituents, the ring atoms of the heteroaryleno bridge replace the bridging ring atoms of the structure. As an example, consider the following structure:

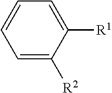

wherein R¹, when taken alone is hydrogen, or when taken together with R² is 5-14 membered heteroaryleno; and R², when taken alone is hydrogen, or when taken together with R¹ is 5-14 membered heteroaryleno.

When R¹ and R² are each hydrogen, the resultant compound is benzene. When R1 taken together with R² is a 6-membered heteroaryleno pyridino), the resultant compound is isoquinoline, quinoline or quinolizine. When R¹ taken together with R² is a 10-membered heteroaryleno (e.g., isoquinoline), the resultant compound is, e.g., acridine or phenanthridine. Typical heteroaryleno groups include, but are not limited to, acridino, carbazolo, β-carbolino, chromeno, cinnolino, furan, imidazolo, indazoleno, indoleno, indolizino, isobenzofurano, isochromeno, isoindoleno, isoquinolino, isothiazoleno, isoxazoleno, naphthyridino, oxadiazoleno, oxazoleno, perimidino, phenanthridino, phenanthrolino, phenazino, phthalazino, pteridino, purino, pyrano, pyrazino, pyrazoleno, pyridazino, pyridino, pyrimidino, pyrroleno, pyrrolizino, quinazolino, quinolino, quinolizino, quinoxalino, tetrazoleno, thiadiazoleno, thiazoleno, thiopheno, triazoleno, xantheno, or the like. Where a specific connectivity is intended, the involved bridging atoms (of the heteroaryleno bridge) are denoted in brackets, e.g., [1,2]pyridino, [2,3]pyridino, [3,4]pyridino, etc. Thus, in the above example, when R¹ taken together with R² is [1,2]pyridino, the resultant compound is quinolizine. When R¹ taken together with R2 is [2,3]pyridino, the resultant compound is quinoline. When R¹ taken together with R² is [3,4]pyridino, the resultant compound is isoquinoline. In preferred embodiments, the heteroaryleno group is 5-14 membered heteroaryleno or 5-10 membered heteroaryleno. Some preferred heteroaryleno radicals are those derived from parent heteroaromatic ring systems in which any ring heteroatoms are nitrogens, such as imidazolo, indolo, indazolo, isoindolo, naphthyridino, pteridino, isoquinolino, phthalazino, purino, pyrazolo, pyrazino, pyridazino, pyndmo, pyrrolo, quinazolino, quinolino, etc.

The term “heteroaryl-heteroaryl” as used herein refers to a monovalent heteroaromatic radical derived by the removal of one hydrogen atom from a single atom of a ring system in which two or more identical or non-identical parent heteroaromatic ring systems are joined directly together by a single bond, where the number of such direct ring junctions is one less than the number of parent heteroaromatic ring systems involved. Typical heteroaryl-heteroaryl groups include, but are not limited to, bipyridyl, tripyridyl, pyridylpurinyl, bipurinyl, etc. When the number of ring atoms are specified, the numbers refer to the number of atoms comprising each parent heteroaromatic ring systems. For example, 5-14 membered heteroaryl-heteroaryl is a heteroaryl-heteroaryl group in which each parent heteroaromatic ring system comprises from 5 to 14 atoms, e.g., bipyridyl, tripyridyl, etc. In some embodiments, each parent heteroaromatic ring system is independently a 5-14 membered heteroaromatic, more preferably a 5-10 membered heteroaromatic. Also preferred are heteroaryl-heteroaryl groups in which all of the parent heteroaromatic ring systems are identical. Some preferred heteroaryl-heteroaryl radicals are those in which each heteroaryl group is derived from parent heteroaromatic ring systems in which any ring heteroatoms are nitrogens, such as imidazole, indole, indazole, isoindole, naphthyridine, pteridine, isoquinoline, phthalazine, purine, pyrazole, pyrazine, pyridazine, pyridine, pyrrole, quinazoline, quinoline, etc.

The term “biheteroaryl” as used herein refers to a heteroaryl-heteroaryl radical having two identical parent heteroaromatic ring systems joined directly together by a single bond. Typical biheteroaryl groups include, but are not limited to, bipyridyl, bipurinyl, biquinolinyl, and the like. In some embodiments, the heteroaromatic ring systems are 5-14 membered heteroaromatic rings or 5-10 membered heteroaromatic rings. Some preferred biheteroaryl radicals are those in which the heteroaryl groups are derived from a parent heteroaromatic ring system in which any ring heteroatoms are nitrogens, such as biimidazolyl, biindolyl, biindazolyl, biisoindolyl, binaphthyridinyl, bipteridinyl, biisoquinolinyl, biphthalazinyl, bipurinyl, bipyrazolyl, bipyrazinyl, bipyridazinyl, bipyridinyl, bipyrrolyl, biquinazolinyl, biquinolinyl, etc.

The term “heteroarylalkyl” as used herein refers to an acyclic alkyl radical in which one of the hydrogen atoms bonded to a carbon atom, typically a terminal or sp2 carbon atom, is replaced with a heteroaryl radical. Where specific alkyl moieties are intended, the nomenclature heteroarylalkanyl, heteroarylakenyl and/or heterorylalkynyl is used. In some embodiments, the heteroarylalkyl group is a 6-20 membered heteroarylalkyl, e.g., the alkanyl, alkenyl or alkynyl moiety of the heteroarylalkyl is 1-6 membered and the heteroaryl moiety is a 5-14-membered heteroaryl. In some preferred embodiments, the heteroarylalkyl is a 6-13 membered heteroarylalkyl, e.g., the alkanyl, alkenyl or alkynyl moiety is 1-3 membered and the heteroaryl moiety is a 5-10 membered heteroaryl.

The term “substituted” as used herein refers to a radical in which one or more hydrogen atoms are each independently replaced with the same or different substituent(s). Typical substituents include, but are not limited to, —X, —R, —O—, ═O, —OR, —O—R, —SR, —S—, ═S, —NRR, ═NR, perhalo (C1-C6) alkyl, —CX3, —CF3, —CN, —OCN, —SCN, —NCO, —NCS, —NO, —NO2, ═N2, —N3, —S(O)2O—, —S(O)2OH, —S(O)2R, —C(O)R, —C(O)X, —C(S)R, —C(S)X, —C(O)OR, —C(O)O—, —C(S)OR, —C(O)SR, —C(S)SR, —C(O)NRR, —C(S)NRR and —C(NR)NRR, where each X is independently a halogen (e.g., —F or —Cl) and each R is independently hydrogen, alkyl, alkanyl, alkenyl, alkanyl, aryl, arylalkyl, arylaryl, heteroaryl, heteroarylalkyl or heteroaryl-heteroaryl, as defined herein. The actual substituent substituting any particular group will depend upon the identity of the group being substituted.

The term “solvate” as used herein refers to an aggregate that comprises one or more molecules of a compound of the disclosure with one or more molecules of solvent. Examples of solvents that form solvates include, but are not limited to, water, isopropanol, ethanol, methanol, DMSO, ethyl acetate, acetic acid, and ethanolamine. The term “hydrate” refers to the aggregate or complex where the solvent molecule is water. The solvent may be inorganic solvents such as for example water in which case the solvate may be a hydrate. Alternatively, the solvent may be an organic solvent, such as ethanol. Thus, the compounds of the present disclosure may exist as a hydrate, including a monohydrate, dihydrate, hemihydrate, sesquihydrate, trihydrate, tetrahydrate or the like, as well as the corresponding solvated forms. The compound of the disclosure may be true solvates, while in other cases, the compound of the disclosure may merely retain adventitious water or be a mixture of water plus some adventitious solvent.

The term “pharmaceutically acceptable excipient,” as used herein, includes any and all solvents, dispersion media, coatings, antibacterial and antifungal agents, isotonic and absorption delaying agents and the like. The use of such media and agents for pharmaceutical active substances is known in the art, such as in Remington: The Science and Practice of Pharmacy, 20^{th} ed.; Gennaro, A. R., Ed.; Lippincott Williams & Wilkins: Philadelphia, Pa., 2000. Except insofar as any conventional media or agent is incompatible with the active ingredient, its use in the therapeutic compositions is contemplated. Supplementary active ingredients can also be incorporated into the compositions.

The term “solution,” as used herein, refers to a mixture containing at least one solvent and at least one compound that is at least partially dissolved in the solvent.

The term “solvent,” as used herein, means a substance, typically a liquid, that is capable of completely or partially dissolving another substance, typically a solid. Solvents useful in this disclosure include, but are not limited to, water, acetone, methanol, tetrahydrofuran (THF), isopropanol (IPA) or mixtures thereof.

The term “oxysterol” as used herein is meant to encompass one or more forms of oxidized cholesterol. The oxysterols described herein are either independently or collectively active to bone growth in a patient, as described in WO 2013169399 A1, which is hereby incorporated by reference in its entirety.

The oxysterol, sterol or diol can be in a pharmaceutically acceptable salt. Some examples of potentially pharmaceutically acceptable salts include those salt-forming acids and bases that do not substantially increase the toxicity of a compound, such as, salts of alkali metals such as magnesium, potassium and ammonium, salts of mineral acids such as hydrochloride, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, or the like.

Pharmaceutically acceptable salts of oxysterol, sterol or diol include salts prepared from pharmaceutically acceptable non-toxic bases or acids including inorganic or organic bases, inorganic or organic acids and fatty acids. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethyl amine, tripropylamine, tromethamine, and the like. When the compound of the current application is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Fatty acid salts may also be used, e.g., fatty acid salts having greater than 2 carbons, greater than 8 carbons or greater than 16 carbons, such as butyric, caproic, caprylic, capric, lauric, mystiric, palmitic, stearic, arachidic or the like.

In some embodiments, in order to reduce the solubility of the oxysterol, sterol, or diol to assist in obtaining a controlled release depot effect, the oxysterol, sterol, or diol is utilized as the free base or utilized in a salt which has relatively lower solubility. For example, the present application can utilize an insoluble salt such as a fatty acid salt. Representative fatty acid salts include salts of oleic acid, linoleic acid, or fatty acid salts with between 8 to 20 carbons solubility, such as for example, palmeate or stearate.

The term “an OXY133 product” includes OXY133, as well as its polymorphs Forms A, B, C, D, E, F, G, H and I, and solvates or hydrates of OXY133, such as hydrates and those formed with organic solvents.

The term “impurity” is used herein to refer to an impurity of OXY133 or OXY133 monohydrate including diastereomer D1, diastereomer D2 or other OXY133 monohydrate impurity, for example C₂₇H₄₆O₂ diol used to synthesize OXY133 monohydrate or any combinations thereof.

A “therapeutically effective amount” or “effective amount” is such that when administered, the oxysterol (e.g., OXY133), sterol, diol, results in alteration of the biological activity, such as, for example, enhancing bone growth, etc. The dosage administered to a patient can be as single or multiple doses depending upon a variety of factors, including the drug's administered pharmacokinetic properties, the route of administration, patient conditions and characteristics (sex, age, body weight, health, size, etc.), and extent of symptoms, concurrent treatments, frequency of treatment and the effect desired. In some embodiments the formulation is designed for immediate release. In other embodiments the formulation is designed for sustained release. In other embodiments, the formulation comprises one or more immediate release surfaces and one or more sustained release surfaces.

A “depot” includes but is not limited to capsules, microspheres, microparticles, microcapsules, microfibers particles, nanospheres, nanoparticles, coating, matrices, wafers, pills, pellets, emulsions, liposomes, micelles, gels, or other pharmaceutical delivery compositions or a combination thereof. Suitable materials for the depot are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof.

The term “implantable” as utilized herein refers to a biocompatible device (e.g., drug depot) retaining potential for successful placement within a mammal. The expression “implantable device” and expressions of the like import as utilized herein refers to an object implantable through surgery, injection, or other suitable means whose primary function is achieved either through its physical presence or mechanical properties.

“Localized” delivery includes delivery where one or more drugs are deposited within a tissue, for example, a bone cavity, or in close proximity (within about 0.1 cm, or preferably within about 10 cm, for example) thereto. For example, the drug dose delivered locally from the drug depot may be, for example, 10%, 20%, 30%, 40%, 50%, 60%, 70%, 80%, 90%, 95%, 99%, 99.9% or 99.999% less than the oral dosage or injectable dose.

The term “mammal” refers to organisms from the taxonomy class “mammalian,” including but not limited to humans, other primates such as chimpanzees, apes, orangutans and monkeys, rats, mice, cats, dogs, cows, horses, etc.

The oxysterol can be “osteogenic,” where it can enhance or accelerate the ingrowth of new bone tissue by one or more mechanisms such as osteogenesis, osteoconduction and/or osteoinduction.

The term “slurry” or “re-slurry” refers to a crystallization technique wherein a product is dissolved in a solvent in which it has moderate to strong solubility. Subsequently, while stirring, an anti-solvent in which the product has poor solubility is slowly added until the product crystallizes out.

Compositions and methods for preparing OXY133 have been described in International Application No. PCT/2015/064526 filed on Dec. 8, 2015, the contents of which is incorporated herein by reference in its entirety.

New compositions and methods are provided to efficiently and safely make oxysterols including OXY133 and polymorphs of OXY133. Methods and compositions that can efficiently and safely generate OXY133, OXY133 polymorphs and that can be incorporated into pharmaceutical compositions including the same are also provided.

Any of the solid forms of OXY133 polymorphs described herein can be a component of a composition comprising OXY133. In some embodiments, these compositions comprise, consist essentially of or consist of at least one of the solid forms of OXY133 polymorphs described herein are substantially free of other solid forms of OXY133.

The section headings below should not be restricted and can be interchanged with other section headings.

### Oxysterols

The present disclosure includes an osteogenic oxysterol (e.g., OXY133), sterol, or diol and its ability to promote osteogenic differentiation in vitro. OXY133 is a particularly effective osteogenic agent. In various applications, OXY133 is useful in treating conditions that would benefit from localized stimulation of bone formation, such as, for example, spinal fusion, fracture repair, bone regenerative/tissue applications, augmentation of bone density in the jaw for dental implants, osteoporosis or the like. One particular advantage of OXY133 is that it provides greater ease of synthesis and improved time to fusion when compared to other osteogenic oxysterols. OXY133 is a small molecule that can serve as an anabolic therapeutic agent for bone growth, as well as a useful agent for treatment of a variety of other conditions.

One aspect of the application disclosure is a compound, named OXY133, having the formula:

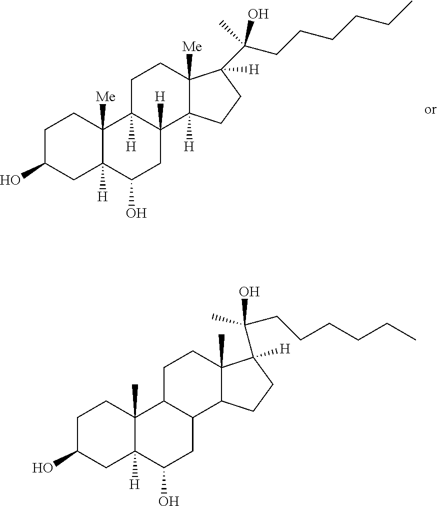

or a pharmaceutically acceptable salt, solvate or hydrate thereof. The OXY133 may be used as a bioactive or pharmaceutical composition comprising OXY133 or a pharmaceutically acceptable salt, solvate or hydrate thereof and a pharmaceutically acceptable carrier.

Another aspect of the disclosure is a method for inducing (stimulating, enhancing) a hedgehog (Hh) pathway mediated response, in a cell or tissue, comprising contacting the cell or tissue with a therapeutically effective amount of OXY133. The cell or tissue can be in vitro or in a subject, such as a mammal. The hedgehog (Hh) pathway mediated response involves the stimulation of osteoblastic differentiation, osteomorphogenesis, and/or osteoproliferation, the stimulation of hair growth and/or cartilage formation; the stimulation of neovasculogenesis, e.g. angiogenesis, thereby enhancing blood supply to ischemic tissues; or it is the inhibition of adipocyte differentiation, adipocyte morphogenesis, and/or adipocyte proliferation; or the stimulation of progenitor cells to undergo neurogenesis. The Hh mediated response can comprise the regeneration of any of a variety of types of tissues, for use in regenerative medicine. Another aspect of the disclosure is a method for treating a subject having a bone disorder, osteopenia, osteoporosis, or a bone fracture, comprising administering to the subject an effective amount of a bioactive composition or pharmaceutical composition comprising OXY133. The subject can be administered the bioactive composition or pharmaceutical composition at a therapeutically effective dose in an effective dosage form at a selected interval to, e.g., increase bone mass, ameliorate symptoms of osteoporosis, reduce, eliminate, prevent or treat atherosclerotic lesions, or the like. The subject can be administered the bioactive composition or pharmaceutical composition at a therapeutically effective dose in an effective dosage form at a selected interval to ameliorate the symptoms of osteoporosis. In some embodiments, a composition comprising OXY133 may include mesenchymal stem cells to induce osteoblastic differentiation of the cells at a targeted surgical area.

In various aspects, the OXY133 can be administered to a cell, tissue or organ by local administration. For example, the OXY133 can be applied locally with a cream or the like, or it can be injected or otherwise introduced directly into a cell, tissue or organ, or it can be introduced with a suitable medical device, such as a drug depot as discussed herein. In some embodiments, the OXY133 can be in an oral formulation, a topical patch, an intranasal or intrapulmonary formulation for inhalation.

In some embodiments, the dosage of OXY133, sterol, or diol is from approximately 10 pg/day to approximately 80 mg/day. Additional dosages of OXY133, sterol, or diol include from approximately 2.4 ng/day to approximately 50 mg/day; approximately 50 ng/day to approximately 2.5 mg/day; approximately 250 ng/day to approximately 250 mcg/day; approximately 250 ng/day to approximately 50 mcg/day; approximately 250 ng/day to approximately 25 mcg/day; approximately 250 ng/day to approximately 1 mcg/day; approximately 300 ng/day to approximately 750 ng/day or approximately 0.50 mcg/day to 500 ng/day. In various embodiments, the dose may be about 0.01 to approximately 10 mcg/day or approximately 1 ng/day to about 120 mcg/day.

In addition to the compound OXY133, sterol, or diol other embodiments of the disclosure encompass any and all individual stereoisomers at any of the stereocenters present in OXY133, including diastereomers, racemates, enantiomers, and other isomers of the compound. In embodiments of the disclosure, OXY133, sterol, oxysterol, diol may include all polymorphs, solvates or hydrates of the compound, such as hydrates and those formed with organic solvents.

The ability to prepare salts depends on the acidity or basicity of a compound. Suitable salts of the compound include, but are not limited to, acid addition salts, such as those made with hydrochloric, hydrobromic, hydroiodic, perchloric, sulfuric, nitric, phosphoric, acetic, propionic, glycolic, lactic pyruvic, malonic, succinic, maleic, fumaric, malic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; salts made with saccharin; alkali metal salts, such as sodium and potassium salts; alkaline earth metal salts, such as calcium and magnesium salts; and salts formed with organic or inorganic ligands, such as quaternary ammonium salts.

Additional suitable salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compounds.

In various embodiments, OXY133, sterol, or diol includes one or more biological functions. That is, OXY133, sterol, or diol can induce a biological response when contacted with a mesenchymal stem cell or a bone marrow stromal cell. For example, OXY133, sterol, or diol may stimulate osteoblastic differentiation. In some embodiments, a bioactive composition including OXY133 sterol, or diol may include one or more biological functions when administered to a mammalian cell, for example, a cell in vitro or a cell in a human or an animal. For example, such a bioactive composition may stimulate osteoblastic differentiation. In some embodiments, such a biological function can arise from stimulation of the hedgehog pathway.

### Methods of Making Intermediary Diol

In some embodiments, the current disclosure provides a method for the preparation of an intermediary diol used in the production of OXY133, as shown below. The diol may be used to promote bone growth as well. Previous methods of synthesis for OXY133 produce were inefficient and not suitable for scale up manufacturing. Some stereoisomers of OXY133 perform less optimally than others. The disclosed method is stereoselective and produces a high yield of the specific isomeric form of the diol shown below, which has been shown to produce an optimally effective isomeric form of OXY133.

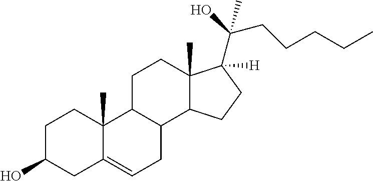 [SMILES: CCCCC[C@](C)(O)[C@H]1CCC2C3CC=C4C[C@@H](O)CC[C@]4(C)C3CC[C@@]21C]

Disclosed are multiple embodiments of reactions to synthesize the intermediary diol. The diol synthesized has the IUPAC designation (3S,8S,9S,10R,13R,14S,17R)-10,13-dimethyl-17-[(S)-2-hydroxyoctan-yl]-2,3,4,7,8,9,11,12,14,15,16,17-dodecahydro-1H-cyclopenta[a]phenanthren-3-ol. Generally, the method of synthesizing the diol includes reacting pregnenolone, pregnenolone acetate or a pregnenolone derivative with an organometallic reagent to facilitate alkylation of the C17 position, as shown below:

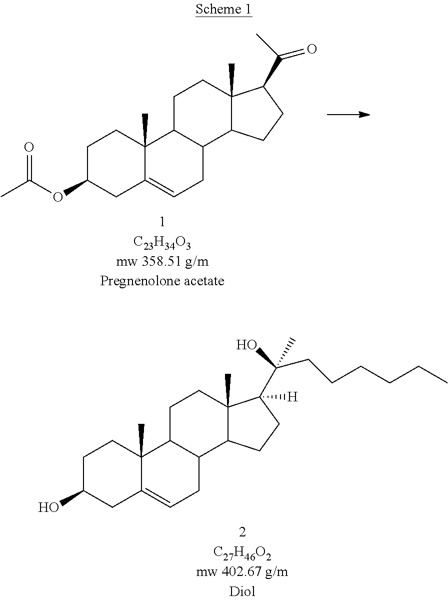 [SMILES: CC(=O)O[C@H]1CC[C@@]2(C)C(=CCC3C2CC[C@@]2(C)C3CC[C@@H]2C(C)=O)C1 ; CCCCCC[C@](C)(O)[C@H]1CCC2C3CC=C4C[C@@H](O)CC[C@]4(C)C3CC[C@@]21C]

In one embodiment, as shown above in Scheme 1, pregnenolone acetate (formula 1) may be alkylated by an organometallic reagent to synthesize the intermediary diol, shown above as formula 2. In some embodiments, pregnenolone acetate is reacted with a Grignard reagent to facilitate alkylation of the C17 position on the pregnenolone acetate molecule. In some embodiments, n-hexylmagnesium chloride is used as the organometallic reagent.

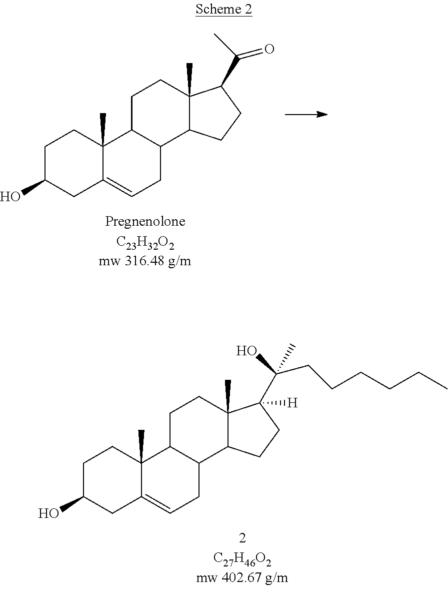 [SMILES: CC(=O)[C@H]1CCC2C3CC=C4C[C@@H](O)CC[C@]4(C)C3CC[C@@]21C ; CCCCCC[C@](C)(O)[C@H]1CCC2C3CC=C4C[C@@H](O)CC[C@]4(C)C3CC[C@@]21C]

In some embodiments, as shown above as Scheme 2, pregnenolone is reacted with a Grignard reagent such as n-hexylmagnesium chloride to facilitate alkylation of the C17 position of the pregnenolone molecule to form the intermediary diol shown as formula 2.

The method of synthesizing the intermediary diol (formula 2) or (3S,8S,9S,10R,13R,14S,17R)-10,13-dimethyl-17-[(S)-2-hydroxyoctan-yl]-2,3,4,7,8,9,11,12,14,15,16,17-dodecahydro-1H-cyclopenta[a]phenanthren-3-ol is stereoselective and produces a high yield of the diol. For example, in some embodiments, the yield of the desired stereoisomer of the diol is between about 60% and about 70%. In some embodiments, the yield of the desired stereoisomer of the diol is between about 50% and about 60%. However, it is contemplated that the percent yield may be higher or lower than these amounts. For example, the percent yield of formula 2 as shown above may be about 20%, 25%, 30%, 35%, 40%, 45%, 50%, 55%, 60%, 65%, 70%, 75%, 80%, 85%, 90% or 95%. In some embodiments, the percent yield may be above 95%.

In various embodiments, the alkylation reaction is carried out in a polar organic solvent, such as tetrahydrofuran. However, the reaction may be carried out in a variety of polar organic solvents. For example, the reaction may be carried out in diethyl ether, ethyl ether, dimethyl ether or the like.

In some embodiments, pregnenolone or pregnenolone acetate is used as a starting reactant. However, in other embodiments, derivatives of pregnenolone acetate may be used. For example, other specific examples of compounds which could be used in the present disclosure include: pregnenolone sulfate, pregnenolone phosphate, pregnenolone formate, pregnenolone hemioxalate, pregnenolone hemimalonate, pregnenolone hemiglutarate, 20-oxopregn-5-en-3β-yl carboxymethyl ether, 3β-hydroxypregn-5-en-20-one sulfate, 3-hydroxy-19-norpregna-1,3,5(10)-trien-20-one, 3-hydroxy-19-norpregna-1,3,5(10),6,8-pentaen-20-one, 17α-isopregnenolone sulfate, 17-acetoxypregnenolone sulfate, 21-hydroxypregnenolone sulfate, 20β-acetoxy-33-hydroxypregn-5-ene-sulfate, pregnenolone sulfate 20-ethyleneketal, pregnenolone sulfate 20-carboxymethyloxime, 20-deoxypregnenolone sulfate, 21-acetoxy-17-hydroxypregnenolone sulfate, 17-propyloxypregnenolone sulfate, 17-butyloxypregnenolone sulfate, 21-thiol esters of pregnenolone sulfate, pyridinium, imidazolium, 6-methylpregnenolone sulfate, 6,16α-dimethylpregnenolone sulfate, 3β-hydroxy-6-methylpregna-5,16-dien-20-one sulfate, 33-hydroxy-6,16-dimethylpregna-5,16-dien-20-one sulfate, 3jβ-hydroxypregna-5,16-dien-20-one sulfate, diosgenin sulfate, 3β-hydroxyandrost-5-en-17β-carboxylic acid methyl ester sulfate, 3a hydroxy-5β-pregnan-20-one formate, 3α-hydroxy-5β-pregnan-20-one hemioxalate, 3α-hydroxy-5β-pregnan-20-one hemimalonate, 3α-hydroxy-5β-pregnan-20-one hemisuccinate, 3α-hydroxy-5β-pregnan-20-one hemiglutarate, estradiol-3-formate, estradiol-3-hemioxalate, estradiol-3-hemimalonate, estradiol-3-hemisuccinate, estradiol-3-hemiglutarate, estradiol-17-methyl ether, estradiol-17-formate, estradiol-17-hemioxalate, estradiol-17-hemimalonate, estradiol-17-hemisuccinate, estradiol-17-hemiglutarate, estradiol-3-methyl ether, 17-deoxyestrone, and 17β-hydroxyestra-1,3,5(10)-trien-3-yl carboxymethyl ether.

In some embodiments, the organometallic comprises n-hexylmagnesium chloride. However, in some embodiments, the alkylation reaction may be carried out with the use of an alkyllithium, such as, for example, n-hexyllithium. In various embodiments, the organometallic includes an alkyl halide. For example, the organometallic reagent may have the following formula:

R—Mg—X,

where Mg comprises magnesium, X comprises chlorine, bromine, fluorine, iodine, or astatine and R comprises an alkyl, a heteroalkyl, an alkanyl, a heteroalkanyl, an alkenyl, a heteroalkenyl, an alkynyl, a heteroalkanyl, an alkyldiyl, a heteroalkyldiyl, an alkyleno, a heteroalkyleno, an aryl, an aryldiyl, an arydeno, an arylaryl, a biaryl, an arylalkyl, a heteroaryl, a heteroaryldiyl, a heteroaryleno, a heteroaryl-heteroaryl, a biheteroaryl, a heteroarylalkyl or combinations thereof. In some embodiments, the R substituent comprises a (C1-C20) alkyl or heteroalkyl, a (C₂-C₂₀) aryl or heteroaryl, a (C₆-C₂₆) arylalkyl or heteroalkyl and a (C₅-C₂₀) arylalkyl or heteroaryl-heteroalkyl, a (C₄-C₁₀) alkyldiyl or heteroalkyldiyl, or a (C₄-C₁₀) alkyleno or heteroalkyleno. The R substituent may be cyclic or acyclic, branched or unbranched, substituted or unsubstituted, aromatic, saturated or unsaturated chains, or combinations thereof. In some embodiments, the R substituent is an aliphatic group. In some embodiments, the R substituent is a cyclic group. In some embodiments, the R substituent is a hexyl group.

Alternatively, the organometallic may comprise the formula:

R-Li,

where Li comprises lithium and R comprises an alkyl, a heteroalkyl, an alkanyl, a heteroalkanyl, an alkenyl, a heteroalkenyl, an alkynyl, a heteroalkanyl, an alkyldiyl, a heteroalkyldiyl, an alkyleno, a heteroalkyleno, an aryl, an aryldiyl, an arydeno, an arylaryl, a biaryl, an arylalkyl, a heteroaryl, a heteroaryldiyl, a heteroaryleno, a heteroaryl-heteroaryl, a biheteroaryl, a heteroarylalkyl or combinations thereof. In some embodiments, the R substituent comprises a (C₁-C₂₀) alkyl or heteroalkyl, a (C₂-C₂₀) aryl or heteroaryl, a (C₆-C₂₆) arylalkyl or heteroalkyl and a (C₅-C₂₀) arylalkyl or heteroaryl-heteroalkyl, a (C₄-C₁₀) alkyldiyl or heteroalkyldiyl, or a (C₄-C₁₀) alkyleno or heteroalkyleno. The R substituent may be cyclic or acyclic, branched or unbranched, substituted or unsubstituted, aromatic, saturated or unsaturated chains, or combinations thereof. In some embodiments, the R substituent is an aliphatic group.

In some embodiments, the R substituent is a cyclic group. In some embodiments, the R substituent is a hexyl group.

In some embodiments, the alkylation reaction is exothermic and the reaction vessel may be temperature controlled to maintain optimal reaction kinetics. In some embodiments, the exothermic reaction releases about 1000 BTU per pound of solution. Due to the strongly exothermic nature of the reaction, the Grignard reagent therefore can be added slowly so that volatile components, for example ethers, are not vaporized due to the reaction heat. In some embodiments, the reaction vessel may be cooled by internal cooling coils. The cooling coils may be supplied with a coolant by means of an external gas/liquid refrigeration unit. In some embodiments, an internal temperature of the reaction vessel is maintained at less than 15° C., 10° C., 5° C. or 1° C. In some embodiments, the reaction vessel is maintained at about 0° C. during the alkylation reaction to form the intermediary diol of formula 2.

In various embodiments, the diol of formula 2 is synthesized along with byproducts and can be purified. For example, the resulting diol of formula 2 may be a byproduct of a diastereomeric mixture. In various embodiments, the diol of formula 2 may be isolated and purified. That is, the diol of formula 2 can be isolated and purified to the desired purity, e.g., from about 95% to about 99.9% by filtration, centrifugation, distillation, which separates volatile liquids on the basis of their relative volatilities, crystallization, recrystallization, evaporation to remove volatile liquids from non-volatile solutes, solvent extraction to remove impurities, dissolving the composition in a solvent in which other components are soluble therein or other purification methods. The diol may be purified by contacting it with organic and/or inorganic solvents, for example, tetrahydrofuran, water, diethyl ether, dichloromethane, ethyl acetate, acetone, n,n-dimethylformamide, acetonitrile, dimethyl sulfoxide, ammonia, t-butanol, n-propanol, ethanol, methanol, acetic acid, or a combination thereof.

In various embodiments, the alkylation step and the purification step take place in the same reaction vessel.

In some embodiments, the diol is quenched with aqueous ammonium chloride or acetic acid to reduce the amount of anions present and neutralize the reaction and separated from the resulting organic layer. The separated residue is recovered by evaporation and purified by silica gel column chromatography.

The diol may be anhydrous or in the monohydrate form. However, in other embodiments the purified diol may be crystallized in other hydrous forms, such as, for example, a dihydrate, a hemihydrate, a sesquihydrate, a trihydrate, a tetrahydrate and the like, as well as the corresponding solvated forms. In other embodiments, the purified diol is crystallized as a co-crystal or a pharmaceutically acceptable salt.

### Methods of Making OXY133

In some embodiments, the current disclosure provides a method for the preparation of an OXY133, as shown below. Previous methods of synthesis for OXY133 produce diastereomeric mixtures of OXY133 intermediates which require purification methods to separate. As discussed above to form the intermediary diol, the disclosed method is stereoselective and produces a high yield of the specific isomeric forms of OXY133. The formula of OXY133 is shown below.

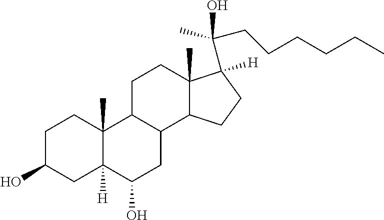 [SMILES: CCCCCC[C@](C)(O)[C@H]1CCC2C3C[C@H](O)[C@H]4C[C@@H](O)CC[C@]4(C)C3CC[C@@]21C]

Disclosed are multiple embodiments of reactions to synthesize OXY133. OXY133 has the IUPAC designation (3S,5S,6S,8R,9S,10R,13S,14S,17S)-17-((S)-2-hydroxyoctan-2-yl)-10,13-dimethylhexadecahydro-1H-cyclopenta[a]phenanthrene-3,6-diol. OXY133 has previously been synthesized through a complex process not suitable for scale-up as shown below:

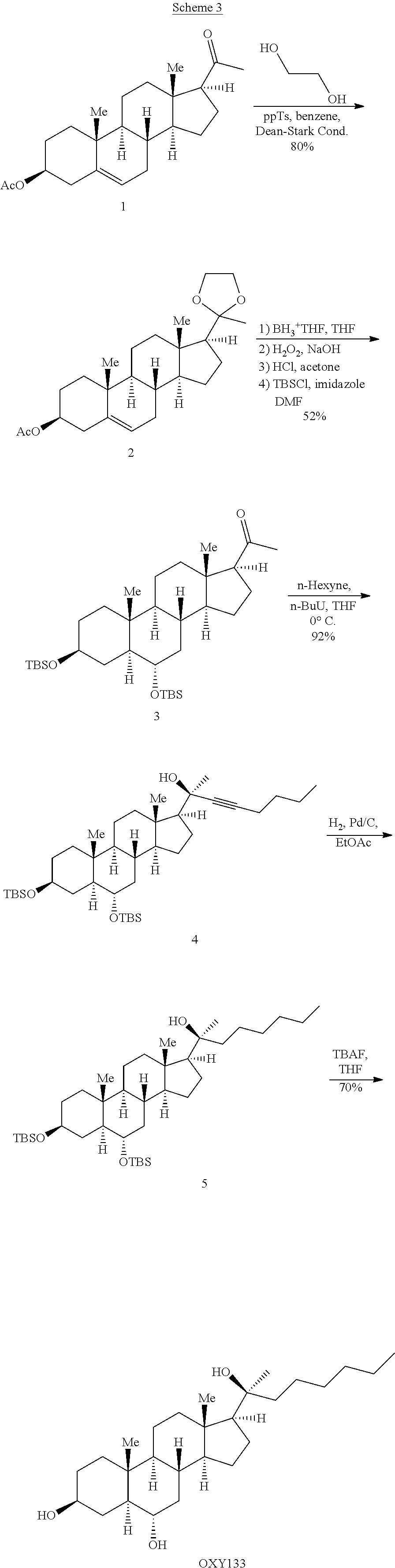

However, the reaction has difficulty being carried out in a single container. The reaction shown above involves more reagents to carry out reaction steps (e.g., blocking and deprotection groups and steps) which have an adverse environmental impact. Additionally, the known methods involve reagents that are expensive and often difficult to obtain. Further, the method shown in Scheme 3 gives relatively low yields, has more degradation products, impurities and creates many toxic byproducts.

Generally, the method of synthesizing OXY133 as disclosed herein includes reacting the diol synthesized as described herein with borane in the reaction shown below:

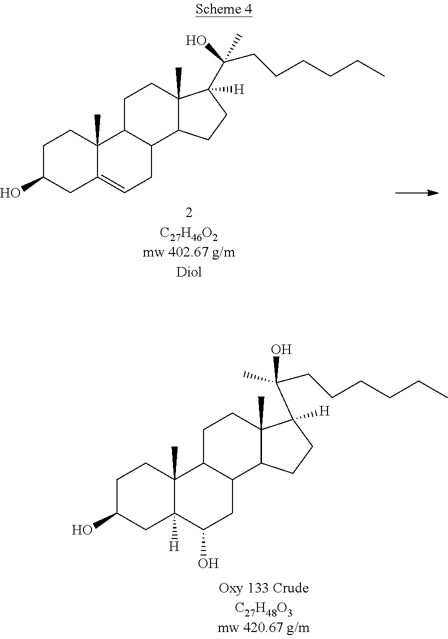 [SMILES: CCCCCC[C@](C)(O)[C@H]1CCC2C3CC=C4C[C@@H](O)CC[C@]4(C)C3CC[C@@]21C ; CCCCCC[C@](C)(O)[C@H]1CCC2C3C[C@H](O)[C@H]4C[C@@H](O)CC[C@]4(C)C3CC[C@@]21C]

In some embodiments, crude and unpurified OXY133 is produced through a hydroboration and oxidation reaction of the intermediary diol having formula 2 in reaction scheme 4. Borane compounds that can be used in the reaction include BH₃, B₂H₆., BH₃S(CH₃)₂ (BMS), borane adducts with phosphines and amines, e.g., borane triethylamine; monosubstituted boranes of the form RBH₂ where R=alkyl and halide, monoalkyl boranes (e.g., IpcBH2, monoisopinocampheylborane), monobromo- and monochloro-borane, complexes of monochloroborane and 1,4-dioxane, disubstituted boranes including bulky boranes, such as for example, dialkylborane compounds such as diethylborane, bis-3-methyl-2-butylborane (disiamylborane), 9-borabycyclo[3,3,1]nonane (9-BBN), disiamylborane (Sia2BH), dicyclohexylborane, Chx2BH, trialkylboranes, dialkylhalogenoboranes, dimesitylborane (C₆H₂Me₃)₂BH, alkenylboranes, pinacolborane, or catecholborane or a combination thereof.

Briefly, a hydroboration and oxidation reaction is a two-step reaction. The boron and hydrogen add across the double bond of an alkene to form a complex with the alkene. Thus the boration phase of the reaction is stereoselective and regioselective. The oxidation phase of the reaction involves basic aqueous hydrogen peroxide to furnish a hydroxyl substituent in place of the boron. See Vollhart, K P, Schore, N E, 2007, Organic Chemistry: Structure and Function, Fifth Ed., New York, N.Y., Custom Publishing Company. Thus, the intermediary diol having formula 2 is reacted with borane and hydrogen peroxide to form crude OXY133. In some embodiments, the step of forming crude OXY133 takes place in the same reaction vessel as the alkylation reaction. In other embodiments, the step of forming crude OXY133 takes place in a different reaction vessel as the alkylation reaction.

The hydroboration-oxidation step of the synthesis of OXY133, like the step of forming the intermediary diol, is stereoselective and produces a high yield. For example, in some embodiments, the percent yield of crude OXY133 may be higher or lower than these amounts. For example, the percent yield of formula 2 as shown above may be about 20%, 25%, 30%, 35%, 40%, 45%, 50%, 55%, 60%, 65%, 70%, 75%, 80%, 85%, 90% or 95%. In some embodiments, the percent yield may be above 95%.

In various embodiments, the hydroboration-oxidation reaction is carried out in a polar organic solvent, such as tetrahydrofuran. However, the reaction may be carried out in a variety of polar organic solvents. For example, the reaction may be carried out in diethyl ether, ethyl ether, dimethyl ether or the like.

In some embodiments, the hydroboration-oxidation reaction is exothermic and the reaction vessel can be temperature controlled to maintain optimal reaction kinetics. Specifically, the oxidation phase is extremely exothermic. Due to the strongly exothermic nature of the reaction, the hydrogen peroxide therefore can be added slowly so that volatile components, for example ethers, are not vaporized due to the reaction heat. In some embodiments, the reaction vessel may be cooled by internal cooling coils. The cooling coils may be supplied with a coolant by means of an external gas/liquid refrigeration unit. In some embodiments, an internal temperature of the reaction vessel is maintained at less than 10° C., 5° C., 1° C. or 0° C. In some embodiments, the reaction vessel is maintained at about −5° C. during the hydroboration-oxidation reaction.

In certain embodiments the diol can have a percent crystallinity of a salt, hydrate, solvate or crystalline form of diol to be at least 10%, at least 20%, at least 30%, at least 40%, at least 50%, at least, 60%, at least 70%, at least 80%, at least 90%, at least 95%, or at least 99%. In some embodiments, the percent crystallinity can be substantially 100%, where substantially 100% indicates that the entire amount of diol appears to be crystalline as best can be determined using methods known in the art. Accordingly, therapeutically effective amounts of diol can include amounts that vary in crystallinity. These include instances where an amount of the crystallized diol in a solid form is subsequently dissolved, partially dissolved, or suspended or dispersed in a liquid.

### Purification of OXY133

In some embodiments, the crude OXY133 can be separated from the reaction mixture prior to purification. In some embodiments, an organic solvent such as dichloromethane is added to the crude OXY133 reaction mixture and the resulting organic layer is separated. Once separated, the crude OXY133 exists as a semi-solid viscous mass. The crude OXY133 may be dissolved by any suitable means (e.g., dichloromethane, etc.) and placed into a silica gel column with an organic solvent, such as methanol-ethyl acetate, to solvate the crude OXY133. In some embodiments, the crude OXY133 may be crystallized or recrystallized. In some embodiments, purified OXY133 is formed by recrystallizing the crude OXY133 in a 3:1 mixture of acetone/water, as shown below:

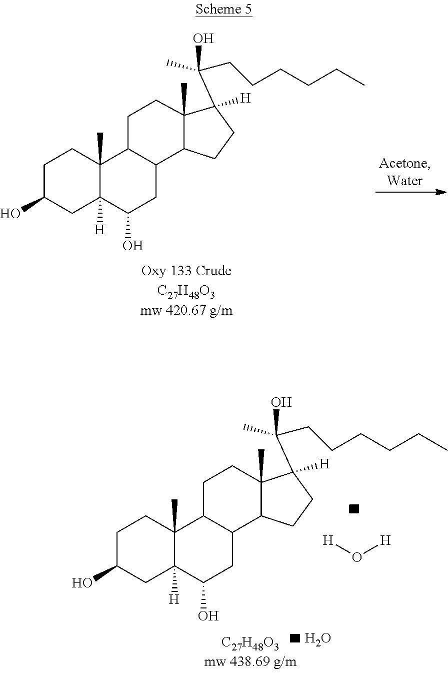

As shown above, upon crystallization, the purified OXY133 forms a hydrate. However, it can be in the anhydrous form, which can be obtained by removing water in ways known in the art. In some embodiments, the percent crystallinity of any of the crystalline forms of OXY133 described herein can vary with respect to the total amount of OXY133.

In certain embodiments the OXY133 can have a percent crystallinity of a salt, hydrate, solvate or crystalline form of OXY133 to be at least 10%, at least 20%, at least 30%, at least 40%, at least 50%, at least, 60%, at least 70%, at least 80%, at least 90%, at least 95%, or at least 99%. In some embodiments, the percent crystallinity can be substantially 100%, where substantially 100% indicates that the entire amount of OXY133 appears to be crystalline as best can be determined using methods known in the art. Accordingly, therapeutically effective amounts of OXY133 can include amounts that vary in crystallinity. These include instances where an amount of the crystallized OXY133 in a solid form is subsequently dissolved, partially dissolved, or suspended or dispersed in a liquid.

In one embodiment, the purified OXY133 is crystallized as a monohydrate. However, in other embodiments the purified OXY133 may be crystallized in other hydrous forms, such as, for example, a dihydrate, a hemihydrate, a sesquihydrate, a trihydrate, a tetrahydrate and the like, as well as the corresponding solvated forms. In other embodiments, the purified OXY133 is crystallized as a co-crystal or a pharmaceutically acceptable salt.

In some embodiments, the reaction mixture containing the crude OXY133 may be solidified by mixing with heptanes. The product may subsequently be filtered and suspended in methylene chloride. In some embodiments, the crude OXY133 may be filtered from the suspension and crystallized with the use of acetone and water or other organic or inorganic solvents (e.g., diethyl ether, dichloromethane, ethyl acetate, acetone, n,n-dimethylformamide, acetonitrile, dimethyl sulfoxide, ammonia, t-butanol, n-propanol, ethanol, methanol, acetic acid or a combination thereof).

In various embodiments, the crude OXY133 may be isolated and purified by any other traditional means. That is, the crude OXY133 can be isolated and purified to the desired purity, e.g., from about 95% to about 99.9% by filtration, centrifugation, distillation to separate volatile liquids on the basis of their relative volatilities, crystallization, recrystallization, evaporation to remove volatile liquids from non-volatile solutes, solvent extraction to remove impurities, dissolving the composition in a solvent in which other components are soluble therein or other purification methods. In various embodiments, the hydroboration-oxidation step and the purification step take place in the same reaction vessel. In various embodiments, the alkylation step, the hydroboration-oxidation step and the purification step take place in the same reaction vessel.

The method of synthesizing the intermediary diol (formula 2) is stereoselective and produces a high yield of OXY133. For example, in some embodiments, the yield of the purified OXY133 is between about 200/% and about 99%. In some embodiments, the yield of the purified OXY133 is between about 20% and about 80%. In some embodiments, the yield of the purified OXY133 is between about 25% and about 70% or about 28%. However, it is contemplated that the percent yield may be higher or lower than these amounts.

In some embodiments, the purified OXY133 is formed in crystal form via crystallization, which separates the OXY133 from the liquid feed stream by cooling the liquid feed stream or adding precipitants which lower the solubility of byproducts and unused reactants in the reaction mixture so that the OXY133 forms crystals. In some embodiments, the solid crystals are then separated from the remaining liquor by filtration or centrifugation. The crystals can be resolubilized in a solvent and then recrystallized and the crystals are then separated from the remaining liquor by filtration or centrifugation to obtain a highly pure sample of OXY133. In some embodiments, the crystals can then be granulated to the desired particle size.

In some embodiments, the crude OXY33 can be purified where the purified OXY133 is formed in crystallized form in a solvent and then removed from the solvent to form a high purity OXY133 having a purity of from about 95% to about 97% or from about 98% to about 99.99%. In some embodiments, the OXY133 can be recovered via filtration or vacuum filtration before or after purification.

### Methods to Making Crystal Polymorphic Forms of OXY133

In certain embodiments, OXY133 anhydrous (polymorph Form B or Form B) can be converted to OXY133 monohydrate (polymorph Form A or Form A) via re-slurry in acetone/water solvent systems. This conversion was slow in that it took about 48 hours and was dependent on the use of the anhydrous OXY133 as the input material.

In other embodiments, several other crystalline forms were produced in various solvent systems when the temperature of the slurry was heated above 30° C. Most of these particular crystalline forms could not be converted to the OXY133 monohydrate via re-slurry.

In order to stop the conversion of OXY133 to polymorph forms other than OXY133 monohydrate (Form A), new solvent systems that would allow for the dissolution of OXY133 at ambient temperatures, below 30° C., followed by precipitation with water were investigated and optimized.

In some embodiments, OXY133 was recrystallized from an acetone/water mixture (3:1) following the reaction scheme 6 below:

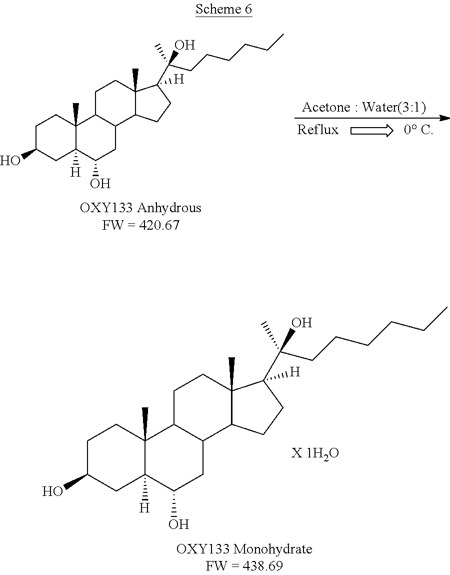 [SMILES: CCCCCC[C@](C)(O)[C@H]1CCC2C3C[C@H](O)[C@H]4C[C@@H](O)CC[C@]4(C)C3CC[C@@]21C ; CCCCCC[C@](C)(O)[C@H]1CCC2C3C[C@H](O)[C@H]4C[C@@H](O)CC[C@]4(C)C3CC[C@@]21C]

In other embodiments, using this procedure OXY133 monohydrate having a theoretical value amount of water present in the solid of about 4.11 wt % was generated. However, in some cases, the OXY133 was isolated as an anhydrous form and as a partial hydrate, for example, a hemihydrate, with varying amounts of water present. Without being bound by theory, it is believed that a higher purity input material of OXY133 could cause the generation of other crystalline forms from the above recrystallization procedure.

In various embodiments, OXY133 anhydrous (Form B) was re-slurried in a slurry-to-slurry conversion in several different solvent systems as summarized in Table 1 below.

TABLE 1

| Solvent System | Temperature (° C.) | Crystal Form |
|---|---|---|
| H₂O | 20 | E |
| H₂O | 70 | E |
| Acetone/H₂O (1:1) | 0 | A |
| Acetone/H₂O (1:1) | 10 | A |
| Acetone/H₂O (1:1) | 20 | A |
| Acetone/H₂O (1:1) | 25 | A |
| Acetone/H₂O (1:1) | 30 | C |
| Acetone/H₂O (1:1) | 40 | C |
| Acetone/H₂O (1:1) | 50 | C |
| Acetone/H₂O (1:1) | 60 | C |
| Acetone/H₂O (1:1) | 70 | C |
| MeOH/H₂O (1:1) | 20 | D |
| MeOH/H₂O (1:1) | 70 | D |

In some embodiments, the temperature of the solvent system can be controlled to obtain the polymorphic form and the temperature can be from about 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, 55.0, 55.5, 56.0, 56.5, 57.0, 57.5, 58.0, 58.5, 59.0, 59.5, 60, 61, 62, 63, 64, 65, 66, 67, 68, 69, to about 70° C. to obtain the polymorph.

As illustrated in Table 1, acetone/water at a ratio of 1:1 yielded Form A after 48 hours of stirring the anhydrous OXY133 slurry at temperatures from about 15° C. to about 25° C. Additionally, stirring under different conditions, for example acetone/water conditions at about 70° C. yielded several new crystalline forms, in particular OXY133 polymorph Forms C, D, and E. While the acetone/water experiments showed that the OXY133 Form B could be converted to Form A, the conversion was rather slow, taking between 24 and 48 hours to convert fully.

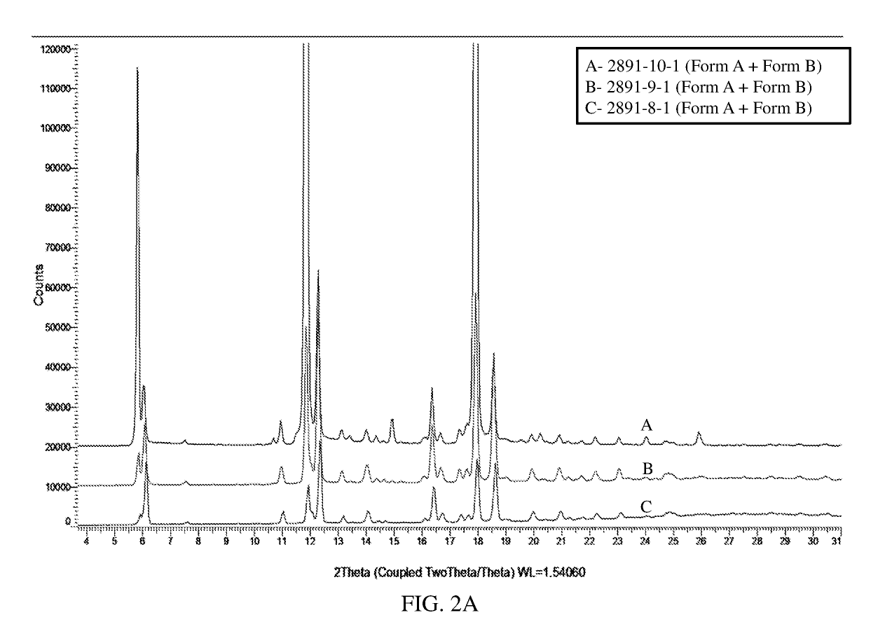

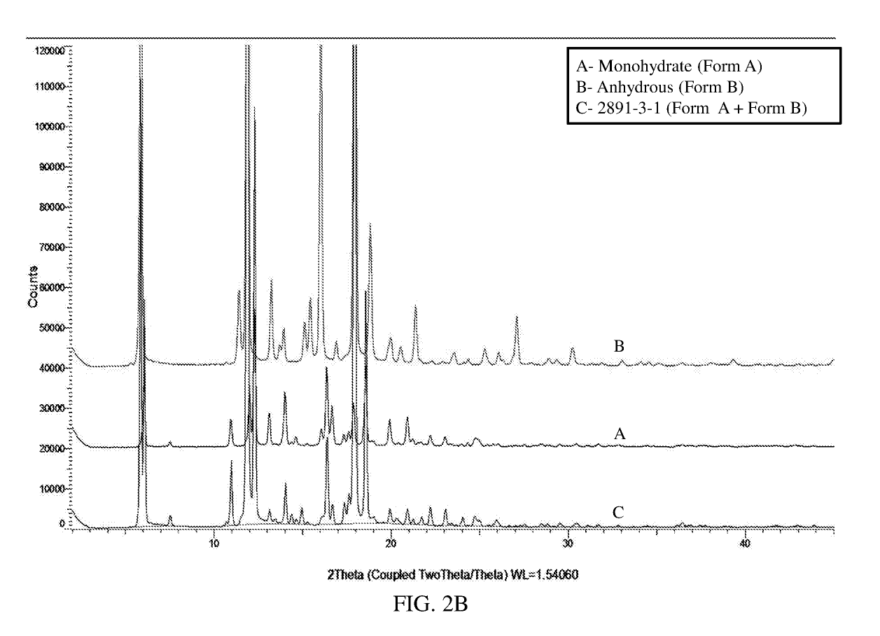

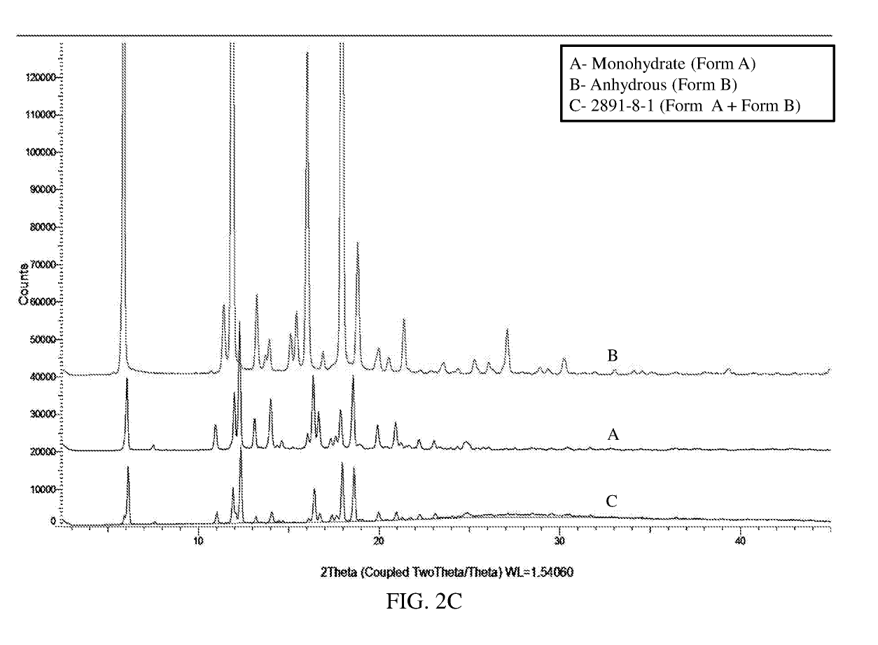

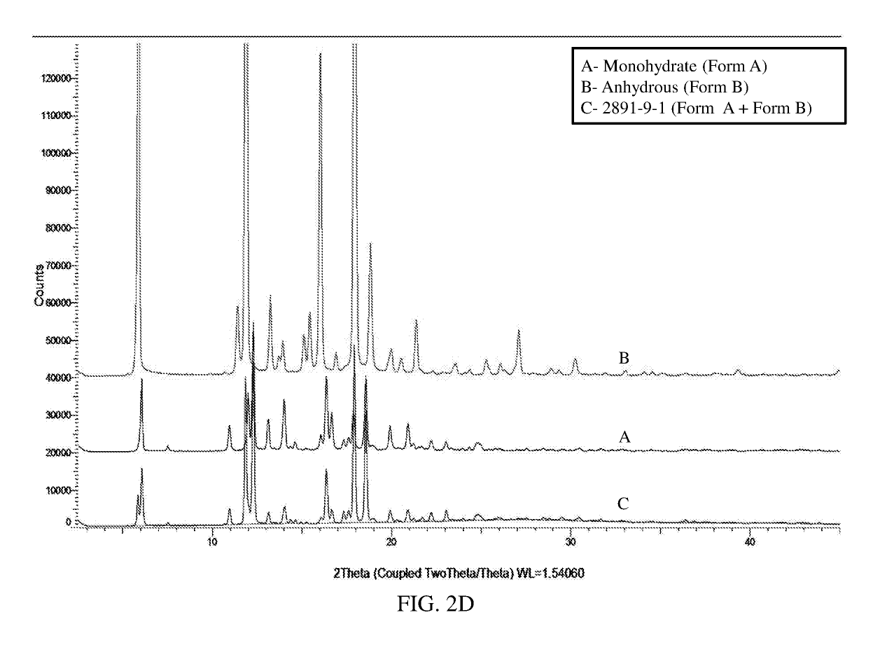

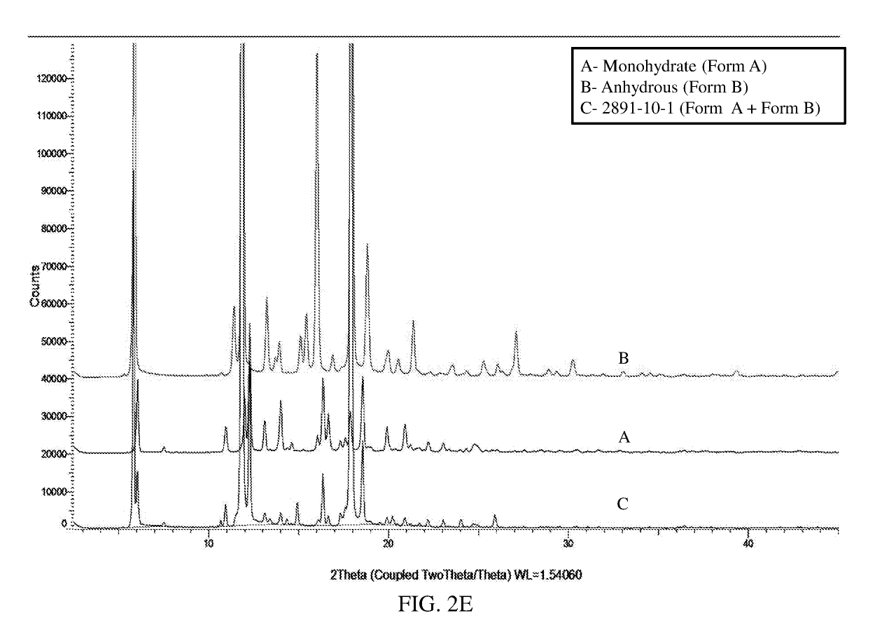

In an effort to improve the rate at which Form B converts to Form A using the re-slurry conditions in acetone/water, the temperature of the slurry was studied. In various embodiments, increasing the slurry temperature to 30° C. and above resulted in the production of Form C. Form C was found to be fairly stable in the acetone/water solvent system. However, no conversion from Form C to Form A or Form B was observed when the slurry was stirred at a temperature of about 20° C. in acetone/water. In other embodiments, since increasing the temperature of the slurry did not yield Form A, temperatures ranging from 0° C. to 25° C. were studied. Surprisingly, the conversion from Form B to Form A was faster with lower temperatures. FIG. 2A shows the difference in XRPDs of a slurry of Form B at the same in-process controls time points after stirring in acetone/water mixtures for 20 minutes. As a result of stirring in acetone/water for the short period of time of 20 minutes, Polymorph Form B was only partially converted to Form A and, as illustrated in FIG. 2A, the XRPDs of the three samples are mixtures of Forms A and B.

At 0° C., most of Form B was successfully converted to Form A. However, it is also important to note that, as illustrated in Table 2 below, the water content measured according to Karl-Fisher (KF) water determination method of the isolated solid increased with increasing slurry temperature between 0° C. and 25° C., even though the XRPD for each sample matches.

TABLE 2

| | Temperature | Crystal | |
|---|---|---|---|
| Lot# | of Slurry (° C.) | Form | KF (Wt %) |
| 2891-8-3 | 0° C. | Form A | 3.25 |
| 2891-9-3 | 10° C. | Form A | 3.83 |
| 2891-10-3 | 25° C. | Form A | 4.02 |

In some embodiments, the crystal form acquired from the first crystallization from acetone/water (3:1) is dependent on purity. The use of this solvent system could lead to crystal forms other than Form B being used as the starting material for the final form conversion, which could result in a failed form conversion attempt.

Accordingly, in some embodiments, crystallization and dissolution/precipitation methods from other acetone/water solvent systems were investigated. Attempts to dissolve OXY133 Form B in acetone showed that the solubility of OXY133 in acetone is fairly low, specifically, about 10 mg/mL at 20° C. and about 80 mg/mL at reflux or 56° C. Additionally, once all solids have been dissolved, they are precipitated from solution rather quickly when cooling to about 54° C. Cooling to 15° C. and charging water to the acetone solvent resulted in the isolation of yet a new crystal form, OXY133 polymorph Form F or Form F. Form F was also shown to be somewhat stable in acetone/water system as it would not convert to any other polymorph forms when stirred at about 20° C. or about 5° C. Due to the low solubility of OXY133 in acetone, we also investigated other solvent compositions with higher solubility of OXY133.

Additional useful solvent systems for dissolving OXY133 included acetone/tetrahydrofuran (THF), methanol/acetone, isopropanol (IPA) and tetrahydrofuran. All of these solvent systems were found to dissolve the OXY133 anhydrous Form B, however, the presence of acetone in the mixtures significantly decreased the solubility of OXY133. Due to the tendency of OXY133 to convert to several other crystal forms while at temperatures elevated above 30° C., it is important to have sufficient solubility at temperatures as low as 0° C. to keep all materials in solution. In addition, to maximize recovery, the solvent system would ideally allow for the use of a reasonable amount of solvent, for example, less than or equal to 10 volumes. Since anhydrous OXY133 or Form B had a good solubility in tetrahydrofuran and isopropanol (IPA) at ambient temperature of about 20° C., solvent systems utilizing tetrahydrofuran/water and IPA/water systems were also investigated.

In some embodiments, OXY133 polymorph Form A was successfully obtained by dissolving Form B in isopropanol or tetrahydrofuran followed by a slow precipitation with water following the reaction scheme 7 below:

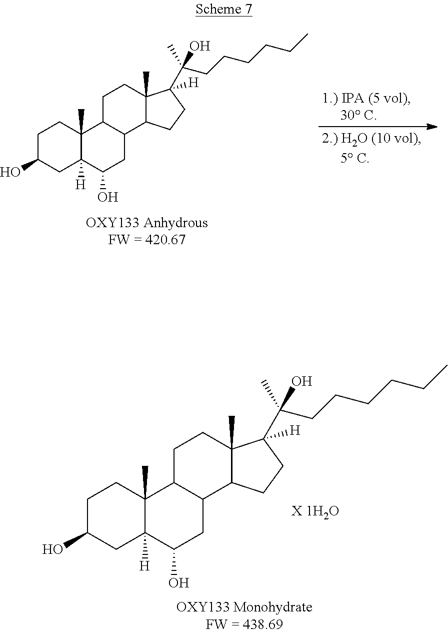 [SMILES: CCCCCC[C@](C)(O)[C@H]1CCC2C3C[C@H](O)[C@H]4C[C@@H](O)CC[C@]4(C)C3CC[C@@]21C ; CCCCCC[C@](C)(O)[C@H]1CCC2C3C[C@H](O)[C@H]4C[C@@H](O)CC[C@]4(C)C3CC[C@@]21C]

While both solvent systems resulted in OXY133 polymorph Form A, the tetrahydrofuran/water system appeared to precipitate OXY133 from solution as an oil upon addition of water to the solution. This oil appeared to convert to a solid after completion of the water addition and stirring for one (1) hour. Accordingly, the isopropanol/water system did not appear to precipitate OXY133 Form A as an oil before solidifying.

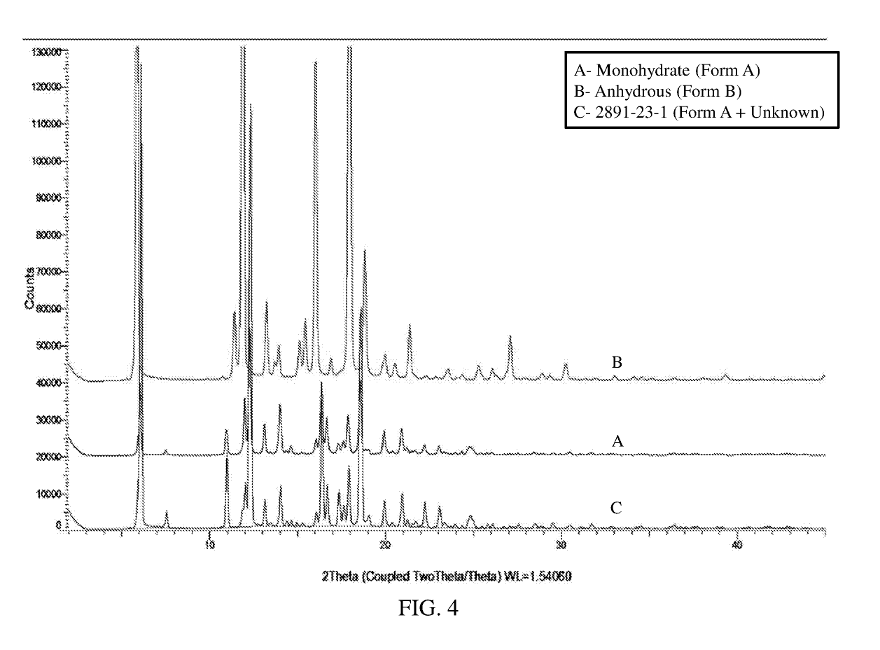

In various aspects of this disclosure, the effects of temperature on the crystal form of OXY133 were investigated in the isopropanol/water system. The optimal working range for the precipitation was found to be about 0° C. to about 10° C., where the target was 5° C. Precipitating OXY133 from IPA with water at temperatures of about 15° C. and about 20° C. also provided OXY133 polymorph Form A. However, precipitation with water at about 30° C. produced what may be a mixture of Form A and a small amount of an additional unknown form as illustrated in FIG. 4. Additionally, in other embodiments, precipitation at −10° C. from isopropanol/water yielded a new polymorph form, OXY133 polymorph Form H or Form H. Overall, the isopropanol/water system had a similar temperature dependence for the precipitation of Form A as the acetone/water system. Table 3 below summarizes the experimental results obtained from isopropanol/water solvent systems at different precipitation temperatures.

TABLE 3

| Solvent System | Precipitation Temperature (° C.) | Crystal Form |
|---|---|---|
| IPA/H₂O (1:1) | 20 | Form A |
| IPA/H₂O (1:2) | 0 | Form A |
| IPA/H₂O (1:2) | 10 | Form A |
| IPA/H₂O (1:2) | 20 | Form A |
| IPA/H₂O (1:2) | 20 | Form A |
| IPA/H₂O (1:2) | 30 | Form A + Unknown |
| IPA/H₂O (1:2) | 40 | Form G |
| IPA/H₂O (1:2) | 5 | Form A |
| IPA/H₂O (1:2) | −10 | Form H |
| IPA/H₂O (1:2) | 5 | Form A |
| IPA/H₂O (1:2) | 5 | Form A |

As illustrated in Table 3 above, the use of isopropanol as the solvent in which Form B was dissolved had several advantages over acetone. The first advantage was being able to keep OXY133 in solution at much lower temperatures, for example about 0° C., avoiding the problem of forming different, stable crystal structures at higher temperatures of greater or equal to 30° C. It also allows for the conversion to Form A from input forms other than Form B, for example, solvates or hemihydrates. Further, the cycle time of the dissolution/precipitation process was short, for example, from about 4 to about 6 hours as opposed to the process of re-slurrying OXY133 in acetone/water for about 48 hours.

The many crystalline forms obtained by subjecting a slurry of OXY133 to different conditions of re-slurrying, recrystallizing from different solvent systems at different temperatures may be identified by many analytical methods, for example, XRPD, HPLC-CAD, DSC-TGA and others known in the art. In certain embodiments, the OXY133 polymorphs may be characterized, at least in part, by X-ray Powder Diffraction (XRPD). In particular, crystalline solids produce a distinctive diffraction pattern of peaks, represented in what is referred to as a diffractogram. The peak assignments for a given crystalline material, for example, degree 2θ values, may vary slightly, depending on the instrumentation used to obtain the diffractogram and certain other factors, for example, sample preparation. Nevertheless, these variations should not be more than +/−0.2 degrees 2θ and the relative spacing between the peaks in the diffractogram will always be the same, regardless of the instrumentation used or the method of sample preparation, and the like.

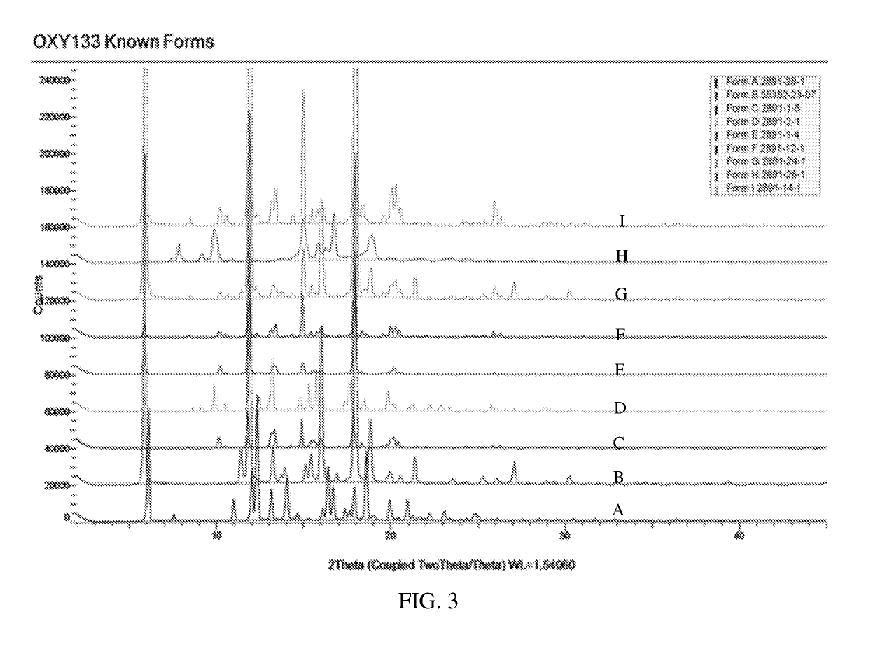

For example, XRPD spectral data relating to the many OXY133 polymorphs are depicted in FIGS. 2-13B. In particular, FIGS. 2A to 2E are XRPDs of OXY133 polymorph Forms A and B. FIG. 3 is a graphic illustration of XRPDs of OXY133 polymorph Forms A, B, C, D, E, F, G, H and I. FIG. 4 is an XRPD of OXY133 polymorph Form A and unknown formed when OXY133 is crystallized from an isopropanol/water solvent/anti-solvent system in a ratio of 1:2 v/v at 30° C.

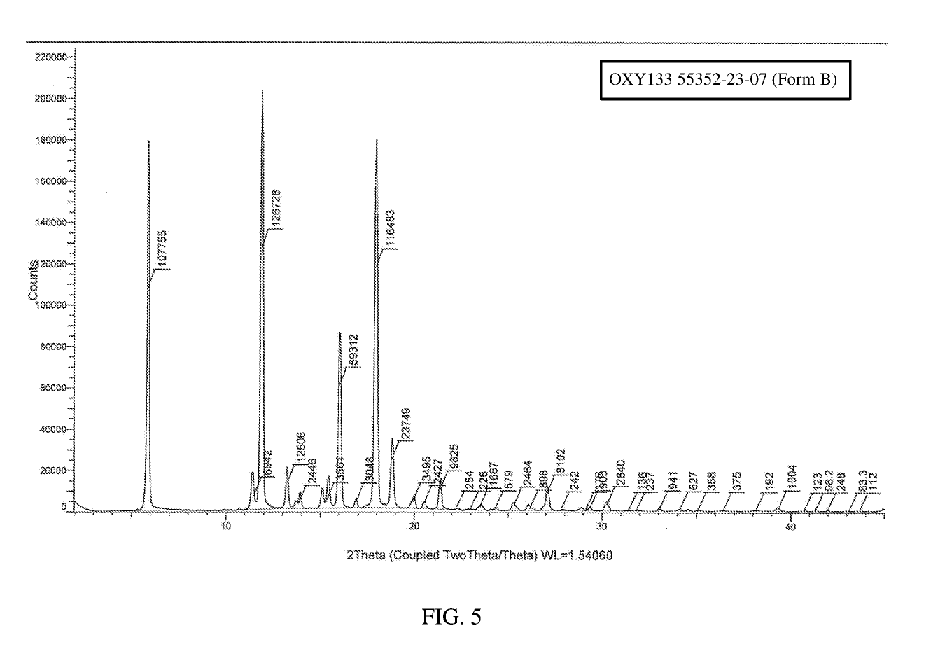

FIG. 5 is an XRPD of OXY133 polymorph Form B or anhydrous OXY133. Table 4, below lists data taken from the XRPD of FIG. 5. As illustrated in Table 4, OXY133 Form B can have one or more reflections of different relative intensities at index numbers 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 and 36.

TABLE 4
XRPD Data for OXY133 Form B, as illustrated in FIG. 5

| | | | Net | Gross | Rel. |
|---|---|---|---|---|---|
| Index | Angle | d Value | Intensity | Intensity | Intensity |
| No. | (2-Theta) | (Angstrom) | (Counts) | (Counts) | (%) |
| 0 | 5.87 | 15.0451 | 107755 | 108530 | 85.00% |
| 1 | 11.469 | 7.70901 | 6942 | 8134 | 5.50% |
| 2 | 11.885 | 7.44037 | 126729 | 128030 | 100.00% |
| 3 | 13.25 | 6.6767 | 12506 | 14054 | 9.90% |
| 4 | 13.842 | 6.39245 | 2446 | 4049 | 1.90% |
| 5 | 15.321 | 5.7786 | 3561 | 5356 | 2.80% |
| 6 | 16.015 | 5.5297 | 59312 | 61227 | 46.80% |
| 7 | 16.905 | 5.24066 | 3048 | 5052 | 2.40% |
| 8 | 17.958 | 4.93554 | 116483 | 118500 | 91.90% |
| 9 | 18.815 | 4.71252 | 23749 | 25703 | 18.70% |
| 10 | 19.925 | 4.45255 | 3495 | 5269 | 2.80% |
| 11 | 20.513 | 4.32619 | 2427 | 4060 | 1.90% |
| 12 | 21.383 | 4.15212 | 9825 | 11229 | 7.80% |
| 13 | 22.2 | 4.00108 | 254 | 1413 | 0.20% |
| 14 | 22.953 | 3.87158 | 226 | 1253 | 0.20% |
| 15 | 23.497 | 3.78317 | 1687 | 2681 | 1.30% |
| 16 | 24.283 | 3.66233 | 579 | 1507 | 0.50% |
| 17 | 25.306 | 3.51666 | 2464 | 3378 | 1.90% |
| 18 | 26.144 | 3.40574 | 898 | 1856 | 0.70% |
| 19 | 27.062 | 3.29227 | 8192 | 9143 | 6.50% |
| 20 | 27.804 | 3.20606 | 242 | 1132 | 0.20% |
| 21 | 29.115 | 3.06462 | 176 | 995 | 0.10% |
| 22 | 29.331 | 3.04256 | 903 | 1722 | 0.70% |
| 23 | 30.264 | 2.95083 | 2840 | 3649 | 2.20% |
| 24 | 31.395 | 2.84712 | 136 | 904 | 0.10% |
| 25 | 31.817 | 2.81029 | 237 | 968 | 0.20% |
| 26 | 33.03 | 2.70977 | 941 | 1593 | 0.70% |
| 27 | 34.101 | 2.62707 | 627 | 1313 | 0.50% |
| 28 | 35.073 | 2.55651 | 358 | 1034 | 0.30% |
| 29 | 36.423 | 2.46473 | 375 | 1018 | 0.30% |
| 30 | 38.147 | 2.35726 | 192 | 890 | 0.20% |
| 31 | 39.344 | 2.28825 | 1004 | 1697 | 0.80% |
| 32 | 40.716 | 2.21425 | 173 | 794 | 0.10% |
| 33 | 41.31 | 2.18376 | 98.2 | 785 | 0.10% |
| 34 | 41.969 | 2.15101 | 248 | 915 | 0.20% |
| 35 | 43.121 | 2.09616 | 83.3 | 751 | 0.10% |
| 36 | 43.669 | 2.07112 | 112 | 818 | 0.10% |

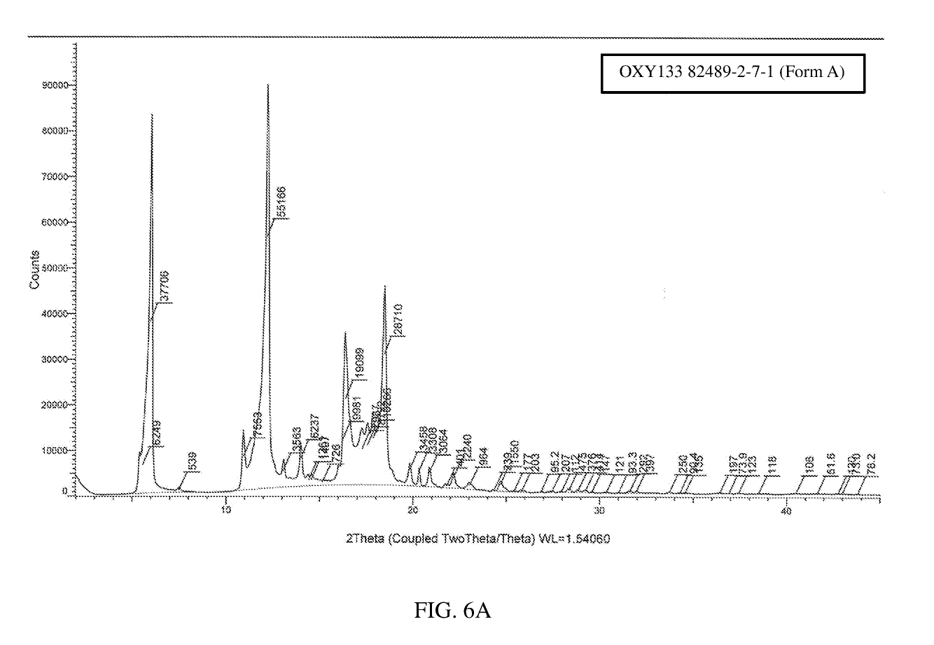

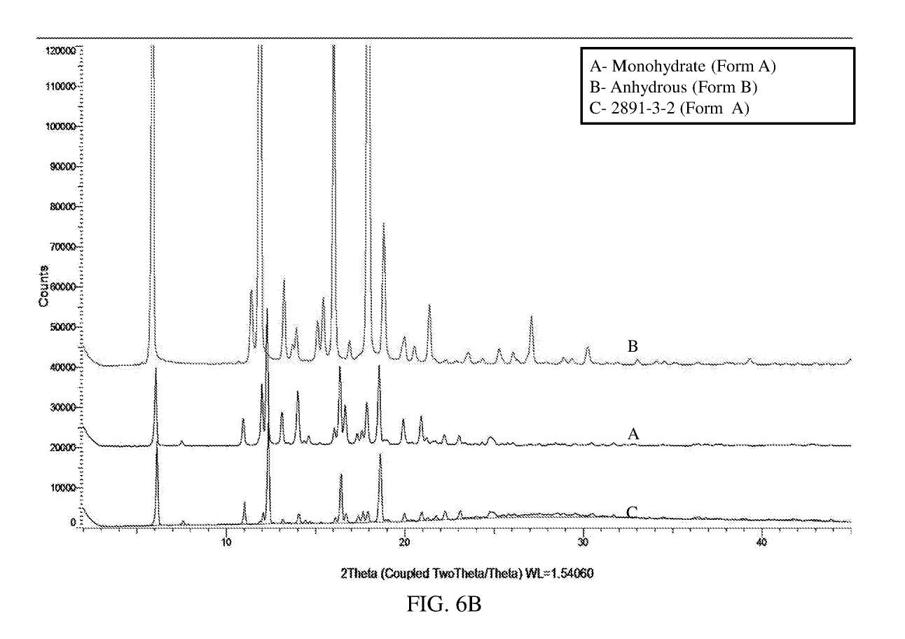

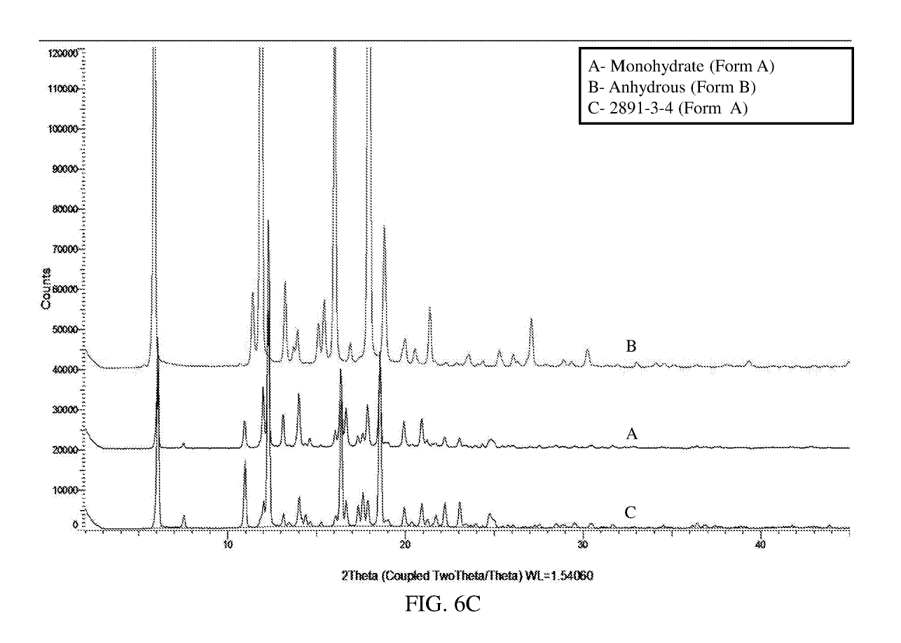

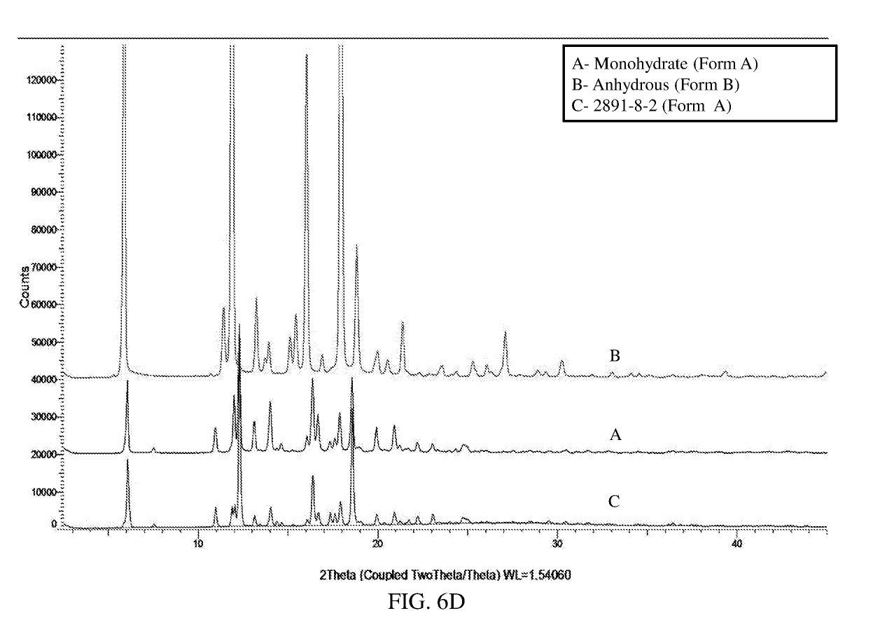

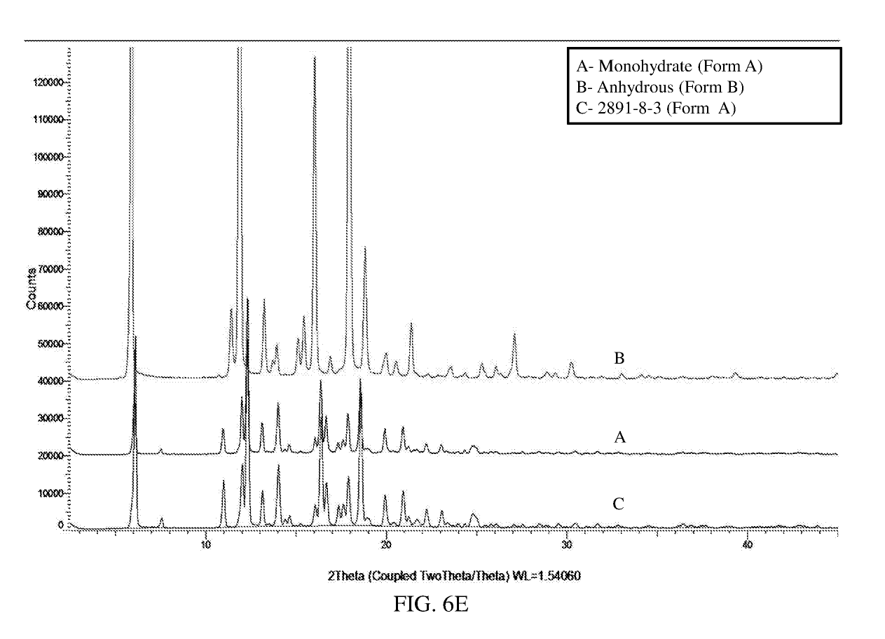

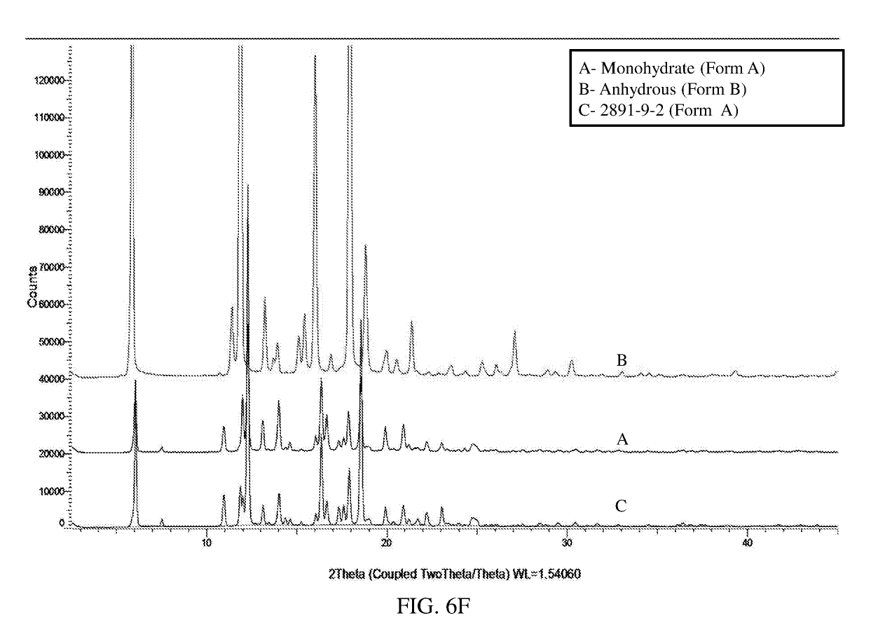

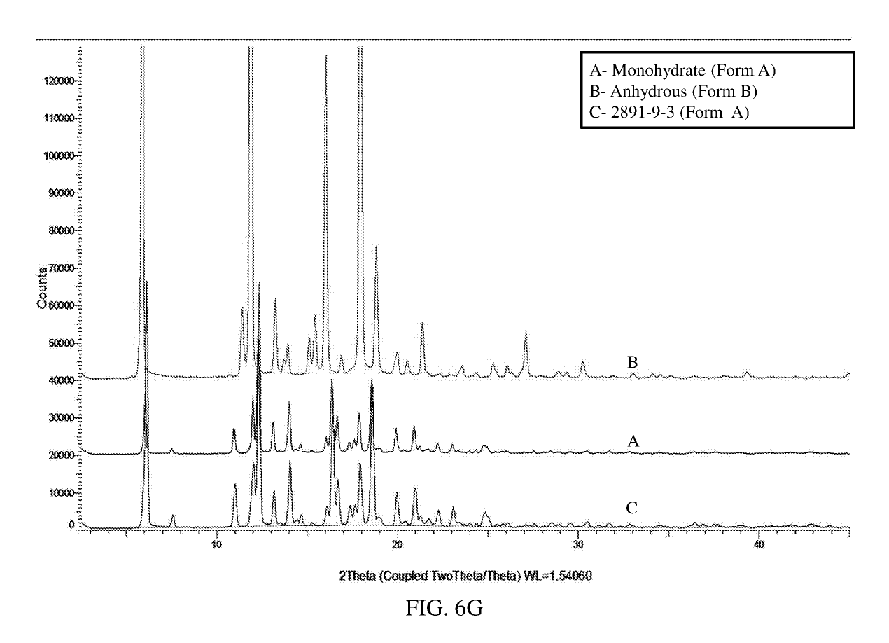

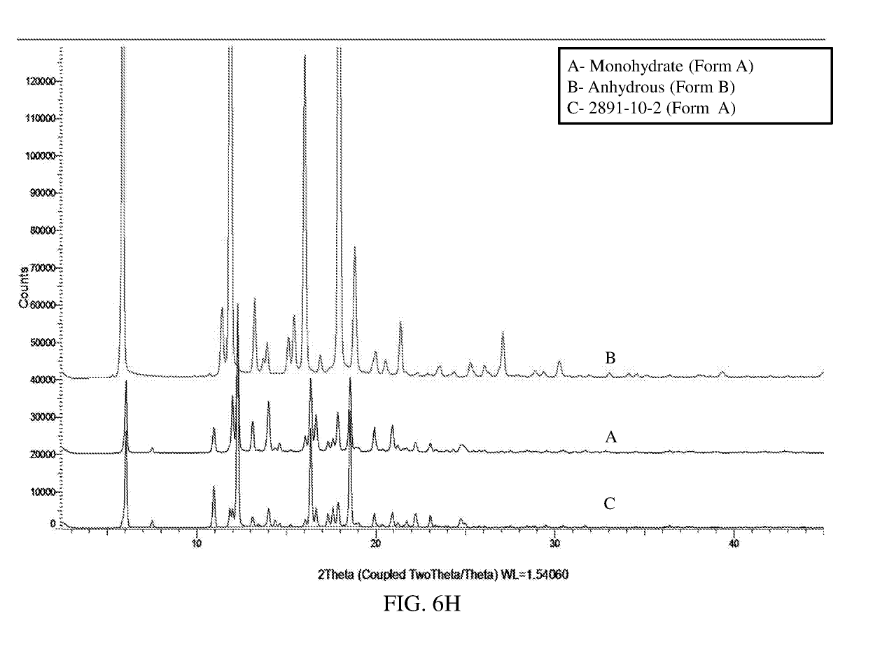

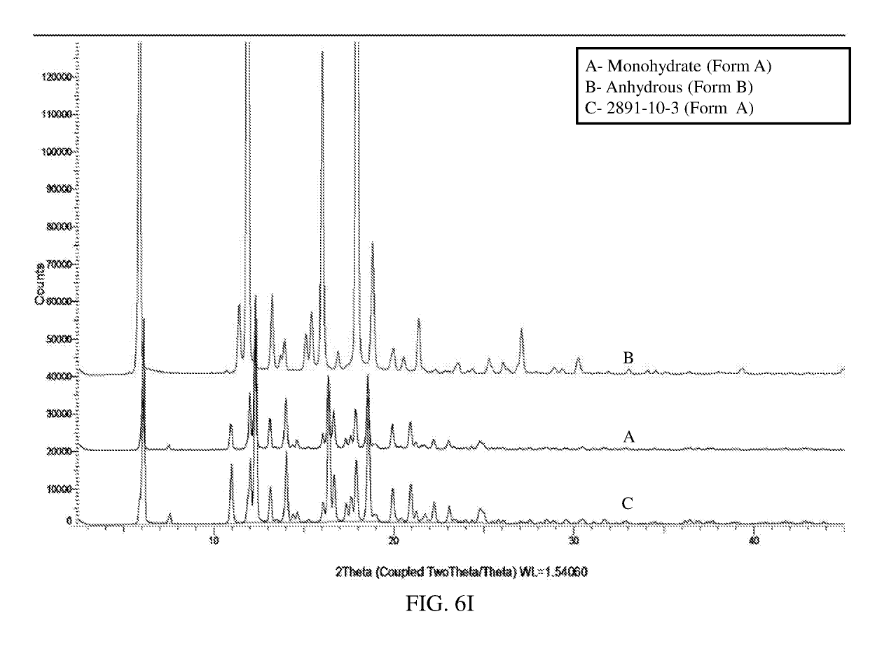

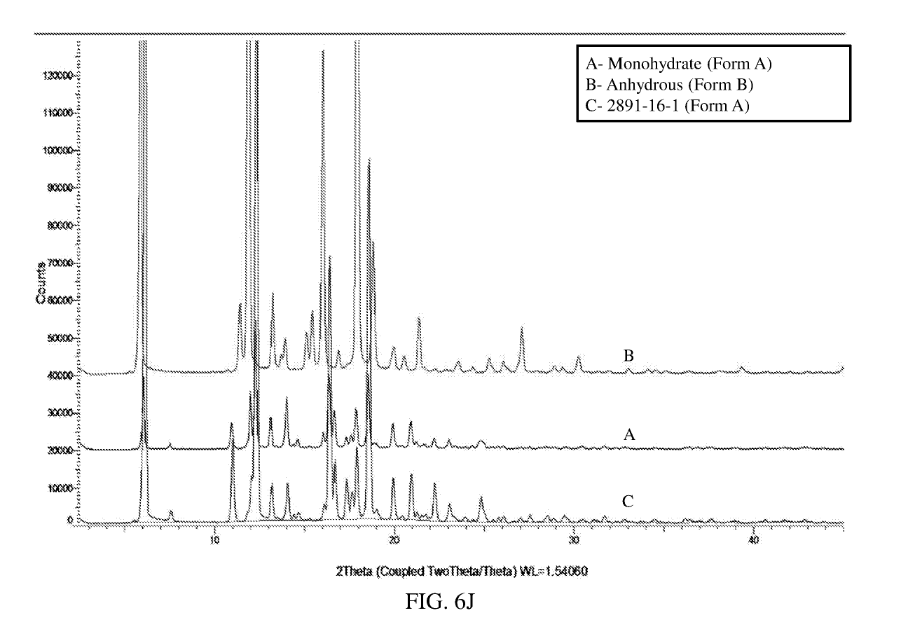

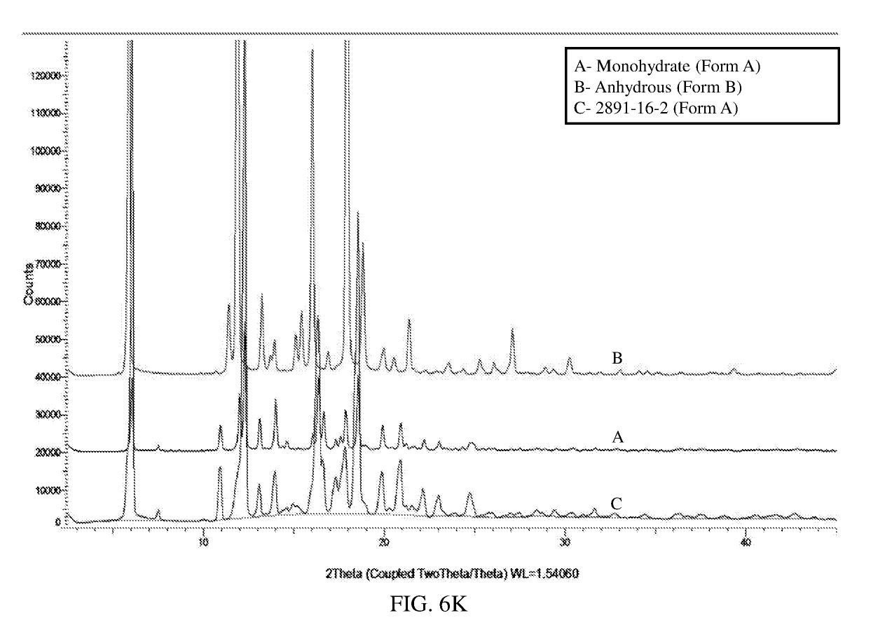

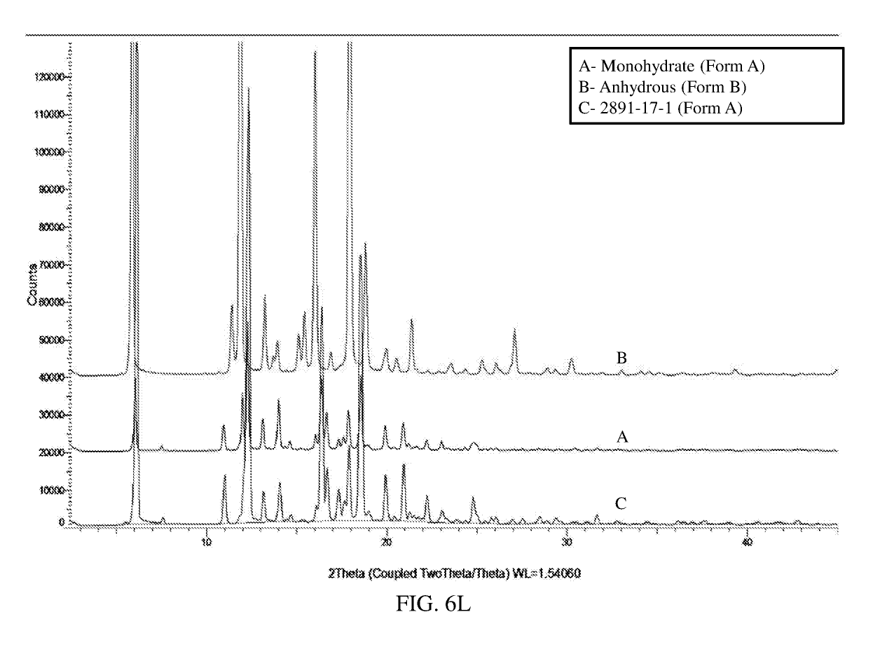

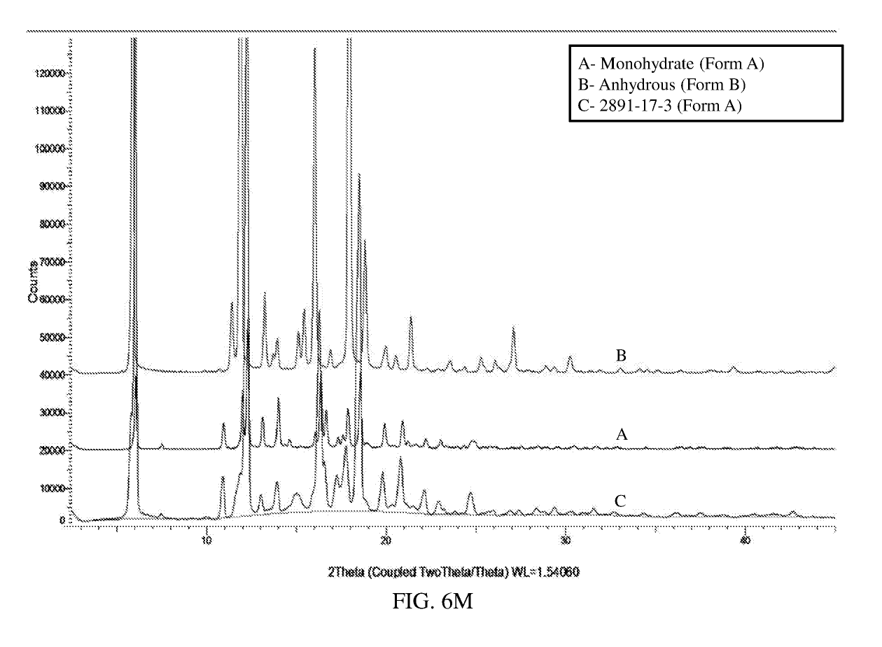

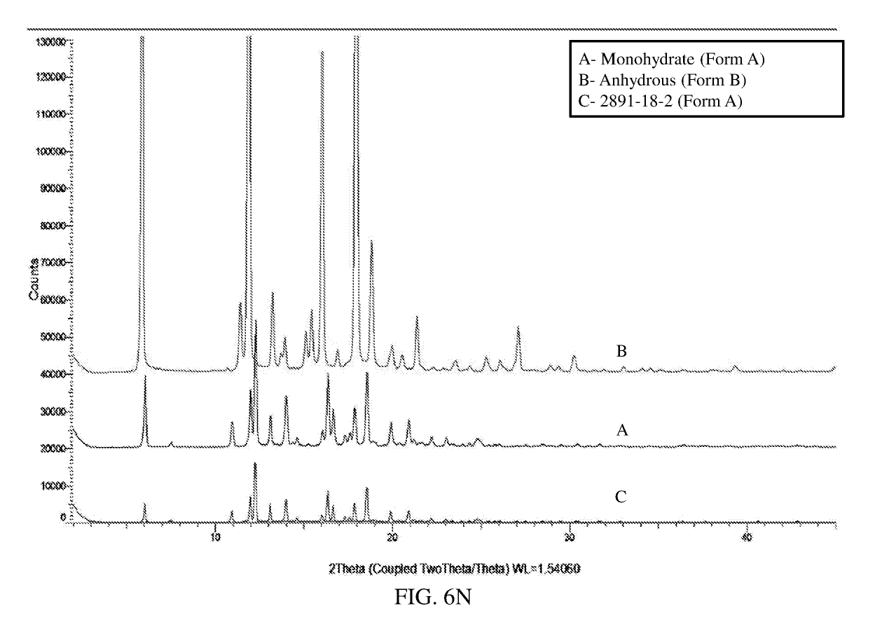

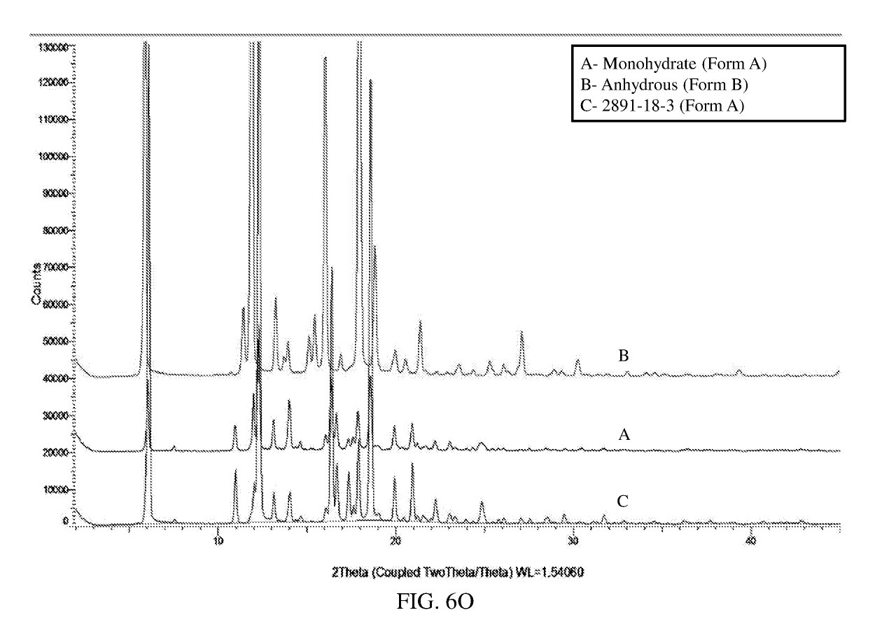

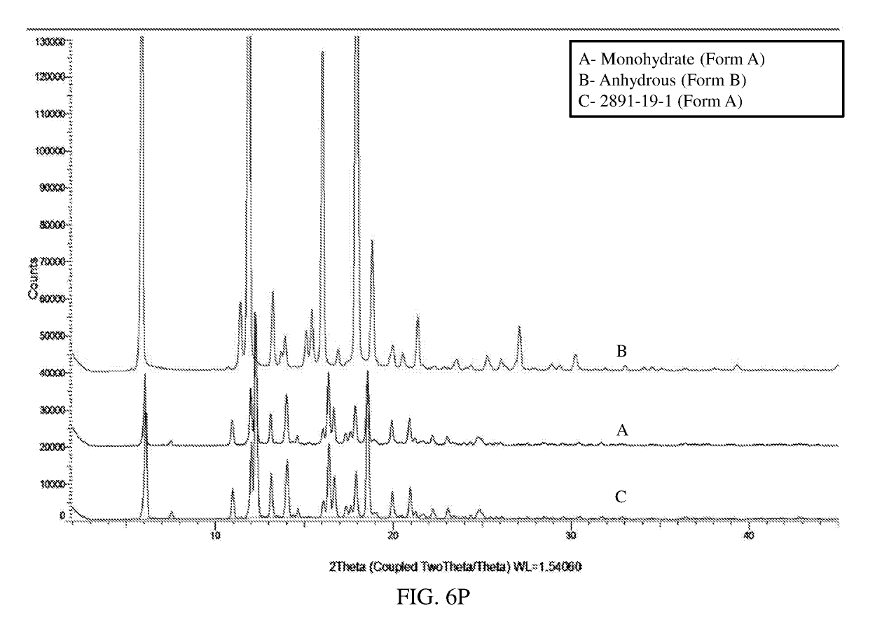

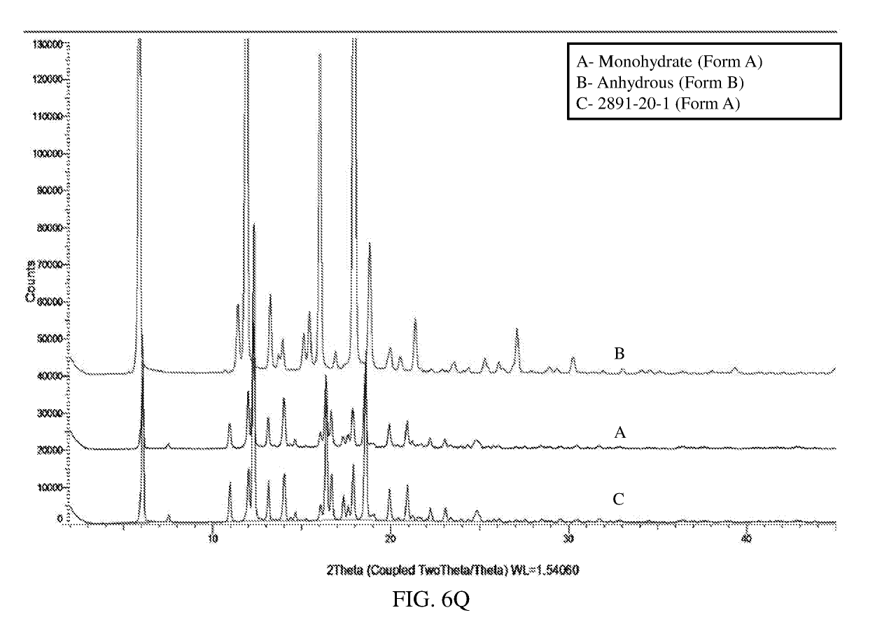

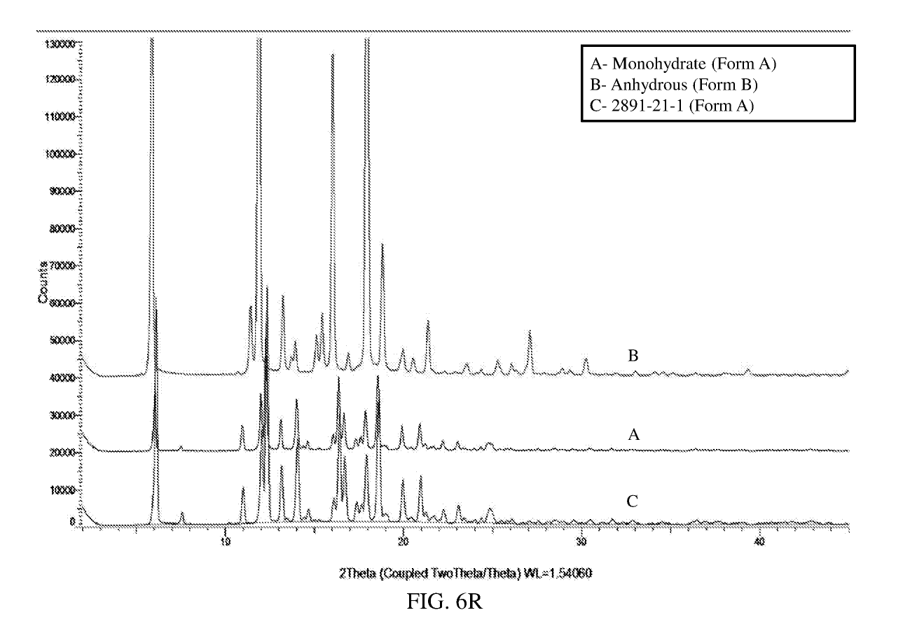

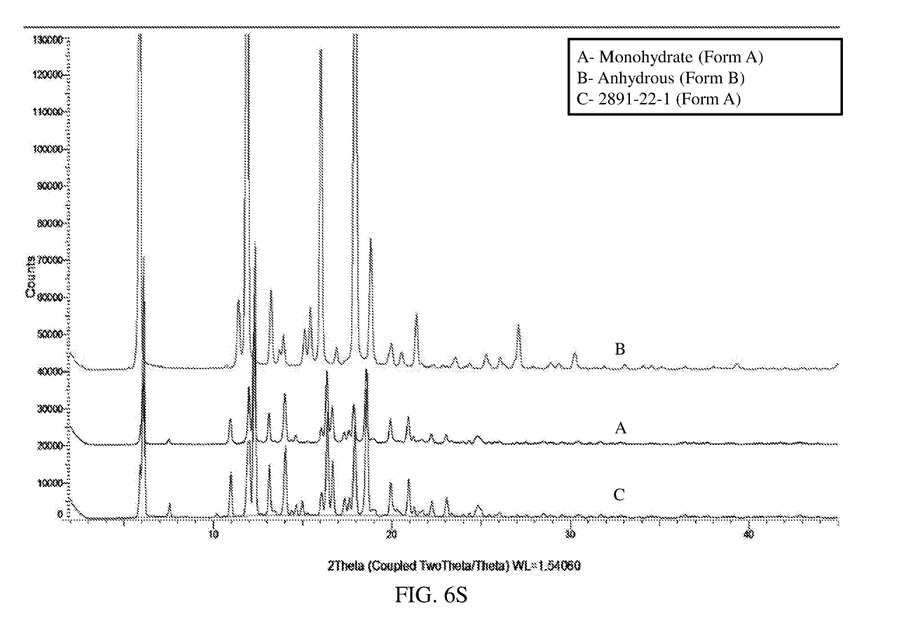

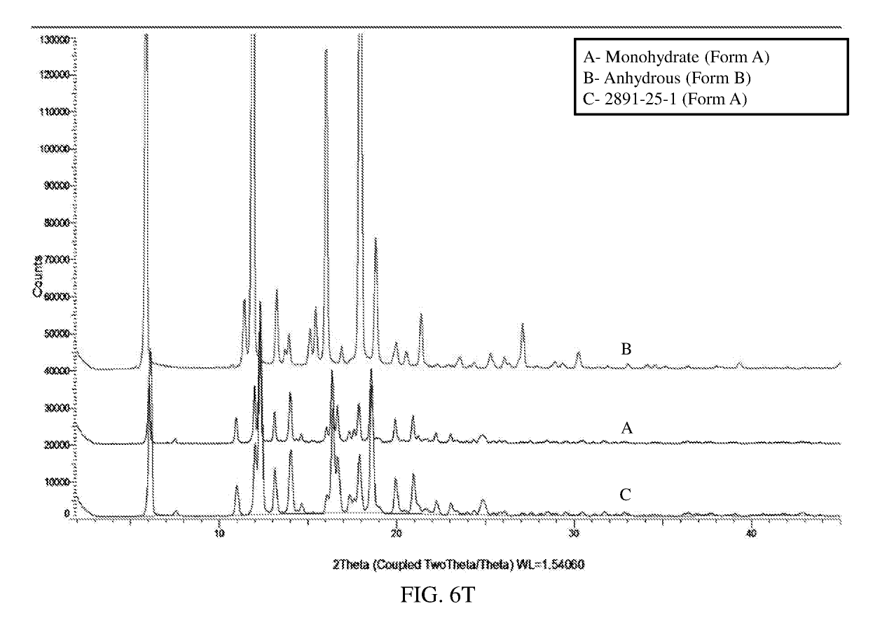

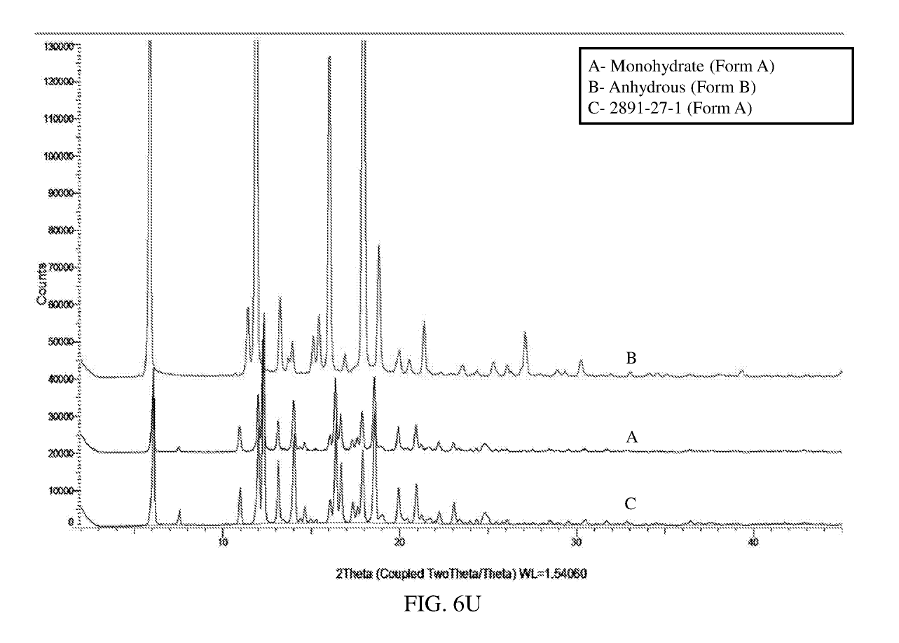

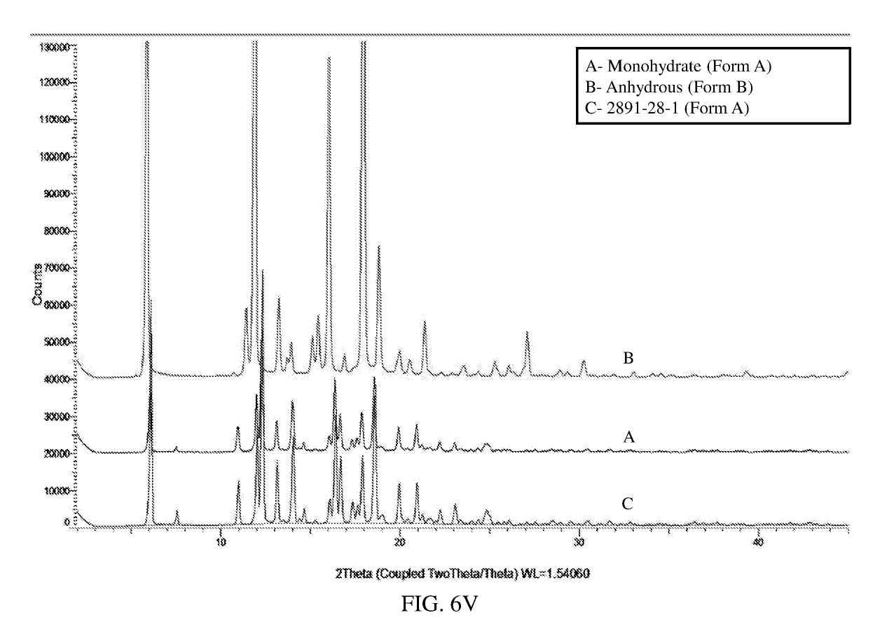

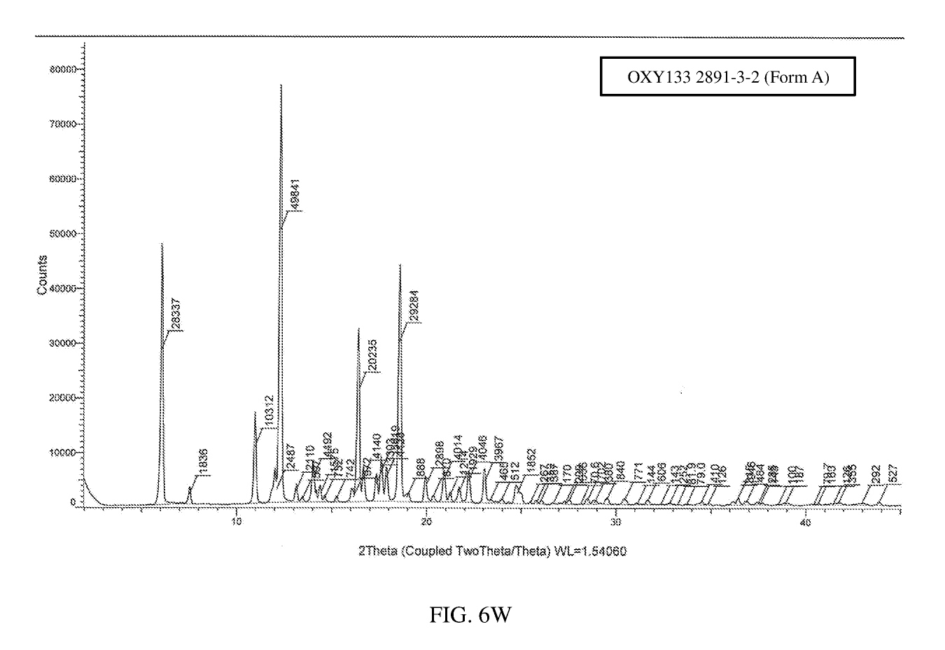

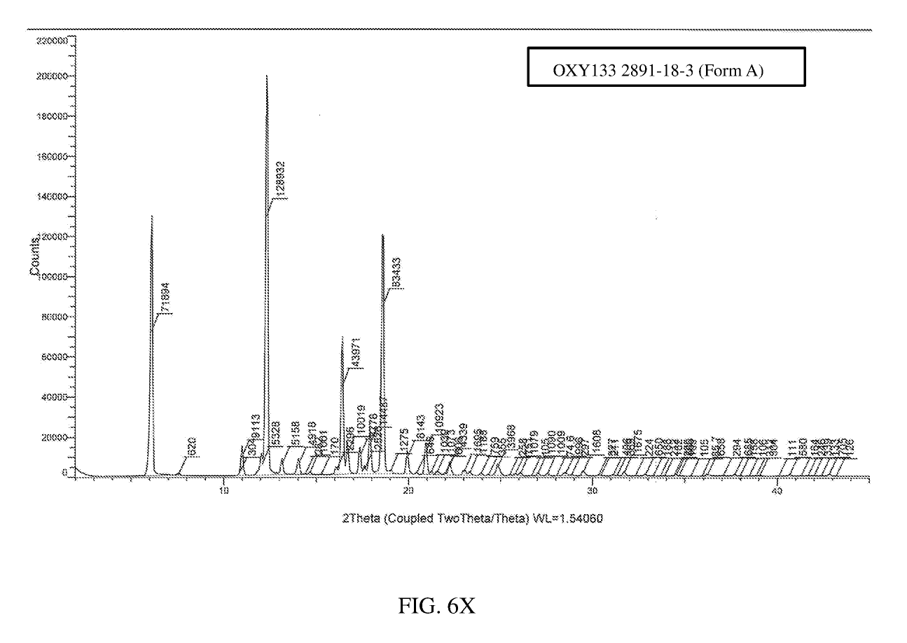

FIGS. 6A, 6B, 6C, 6D, 6E, 6F, 6G, 6H, 6I, 6J, 6K, 6L, 6M, 6N, 60, 6P, 6Q, 6R, 6S, 6T, 6U, 6V, 6W and 6X are XRPDs of OXY133 polymorph Form A. In particular, FIG. 6A is an XRPD of a solid OXY133 Form A. Table 5, below lists data taken from the XRPD of FIG. 6A. As illustrated in Table 5, OXY133 Form A can have one or more reflections of different relative intensities at index numbers 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and 48.

TABLE 5
XRPD Data for OXY133 Form A, as illustrated in FIG. 6A

| | Angle | | Net | Gross | |
|---|---|---|---|---|---|
| Index | (2- | d Value | Intensity | Intensity | Rel. Intensity |
| No. | Theta) | (Angstrom) | (Counts) | (Counts) | (%) |
| 0 | 5.561 | 15.8807 | 6249 | 6906 | 11.30% |
| 1 | 5.967 | 14.799 | 37706 | 38421 | 68.30% |
| 9 | 7.484 | 11.803 | 539 | 1386 | 1.00% |
| 3 | 10.952 | 8.07213 | 7553 | 8876 | 13.70% |
| 4 | 12.183 | 7.25924 | 55166 | 56910 | 100.00% |
| 5 | 13.059 | 6.77383 | 3563 | 5552 | 6.50% |
| 6 | 14.002 | 6.31977 | 6237 | 8436 | 11.30% |
| 7 | 14.423 | 6.13613 | 1361 | 3637 | 2.50% |
| 8 | 14.564 | 6.07711 | 1407 | 3707 | 2.60% |
| 9 | 15.152 | 5.84266 | 726 | 3111 | 1.30% |
| 10 | 16.232 | 5.45628 | 9981 | 12467 | 18.10% |
| 11 | 16.388 | 5.40467 | 19099 | 21594 | 34.60% |
| 12 | 17.278 | 5.12833 | 7987 | 10505 | 14.50% |
| 13 | 17.565 | 5.04494 | 8792 | 11307 | 15.90% |
| 14 | 17.859 | 4.9627 | 10266 | 12773 | 18.60% |
| 15 | 18.447 | 4.80569 | 28710 | 31185 | 52.00% |
| 16 | 19.823 | 4.47519 | 3458 | 5778 | 6.30% |
| 17 | 20.324 | 4.36597 | 3308 | 5543 | 6.00% |
| 18 | 20.885 | 4.24992 | 3064 | 5186 | 5.60% |
| 19 | 21.789 | 4.07571 | 401 | 2307 | 0.70% |
| 20 | 22.173 | 4.00583 | 2240 | 4045 | 4.10% |
| 21 | 23.043 | 3.85655 | 964 | 2507 | 1.70% |
| 22 | 24.351 | 3.65238 | 239 | 1389 | 0.40% |
| 23 | 24.706 | 3.60057 | 1550 | 2669 | 2.80% |
| 24 | 25.419 | 3.50117 | 177 | 1210 | 0.30% |
| 25 | 25.832 | 3.44621 | 203 | 1173 | 0.40% |
| 26 | 26.873 | 3.31496 | 95.2 | 1004 | 0.20% |
| 27 | 27.462 | 3.24523 | 207 | 1132 | 0.40% |
| 28 | 27.964 | 3.18806 | 112 | 1044 | 0.20% |
| 29 | 28.355 | 3.14497 | 475 | 1406 | 0.90% |
| 30 | 28.855 | 3.09163 | 376 | 1294 | 0.70% |
| 31 | 29.247 | 3.05108 | 414 | 1312 | 0.80% |
| 32 | 29.589 | 3.0166 | 147 | 1018 | 0.30% |
| 33 | 30.361 | 2.94166 | 121 | 951 | 0.20% |
| 34 | 31.028 | 2.87987 | 93.3 | 907 | 0.20% |
| 35 | 31.609 | 2.82831 | 292 | 1123 | 0.50% |
| 36 | 31.972 | 2.79704 | 397 | 1229 | 0.70% |
| 37 | 33.731 | 2.65503 | 250 | 1001 | 0.50% |
| 38 | 34.301 | 2.6122 | 90.4 | 829 | 0.20% |
| 39 | 34.568 | 2.59262 | 135 | 860 | 0.20% |
| 40 | 36.421 | 2.46492 | 197 | 911 | 0.40% |
| 41 | 36.926 | 2.43235 | 73.9 | 797 | 0.10% |
| 42 | 37.429 | 2.4008 | 123 | 841 | 0.20% |
| 43 | 38.501 | 2.33638 | 118 | 810 | 0.20% |
| 44 | 40.499 | 2.22559 | 106 | 820 | 0.20% |
| 45 | 41.649 | 2.16678 | 51.6 | 791 | 0.10% |
| 46 | 42.778 | 2.11217 | 130 | 835 | 0.20% |
| 47 | 42.995 | 2.10199 | 73 | 768 | 0.10% |
| 48 | 43.805 | 2.06497 | 78.2 | 715 | 0.10% |

FIGS. 6B-6I are XRPDs of polymorph Form A obtained by re-slurrying from an acetone/water solvent/anti-solvent medium in a ratio of 1:1 v/v at precipitating temperatures of 20° C., 50° C., 0° C., 35° C., 10° C., 25° C., respectively. FIGS. 6J and 6K are XRPDs of polymorph Form A obtained by crystallization from a THF/acetone/water solvent system at temperatures of 35° C. FIGS. 6L and 6M are XRPDs of polymorph Form A by re-slurrying from a THF/water solvent system in a ratio of 1:2 v/v at precipitating temperatures of 20° C. and 35° C., respectively. FIGS. 6N-6V are XRPDs of polymorph Form A obtained by crystallization from IPA/water solvent system in ratio of 1:2 or 1:1 v/v at temperatures of 0° C., 5° C., 10° C. and 20° C., respectively, as also listed in more detail in Table 4 below. In FIG. 6T, the conversion to polymorph Form A is from a hemihydrate of OXY133.

FIG. 6W is an XRPD of polymorph A obtained by re-slurrying from an acetone/water solvent/anti-solvent medium in a ratio of 1:1 v/v at a precipitating temperature of 20° C. FIG. 6W is an XRPD of a solid OXY133 Form A. Table 6, below lists data taken from the XRPD of FIG. 6W. As illustrated in Table 6, OXY133 Form A can have one or more reflections of different relative intensities at index numbers 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 and 61.

TABLE 6
XRPD Data for OXY133 Form A, as illustrated in FIG. 6W

| | | | Net | Gross | Rel. |
|---|---|---|---|---|---|
| Index | Angle | d Value | Intensity | Intensity | Intensity |
| No. | (2-Theta) | (Angstrom) | (Counts) | (Counts) | (%) |
| 0 | 6.066 | 14.55919 | 28337 | 28877 | 56.90% |
| 1 | 7.525 | 11.73815 | 1836 | 2450 | 3.70% |
| 2 | 10.98 | 8.05166 | 10312 | 11050 | 20.70% |
| 3 | 12.116 | 7.29894 | 2487 | 3391 | 5.00% |
| 4 | 12.292 | 7.19472 | 49841 | 50764 | 100.00% |
| 5 | 13.141 | 6.73178 | 2110 | 3103 | 4.20% |
| 6 | 13.467 | 6.56952 | 597 | 1605 | 1.20% |
| 7 | 14.043 | 6.30144 | 4492 | 5514 | 9.00% |
| 8 | 14.413 | 6.1407 | 1575 | 2596 | 3.20% |
| 9 | 14.631 | 6.04969 | 732 | 1748 | 1.50% |
| 10 | 15.261 | 5.80105 | 742 | 1742 | 1.50% |
| 11 | 16.141 | 5.48665 | 972 | 2013 | 1.90% |
| 12 | 16.398 | 5.4013 | 20235 | 21297 | 40.60% |
| 13 | 16.648 | 5.32084 | 4140 | 5219 | 8.30% |
| 14 | 17.36 | 5.1042 | 3303 | 4412 | 6.60% |
| 15 | 17.616 | 5.03065 | 5619 | 6731 | 11.30% |
| 16 | 17.881 | 4.95652 | 4428 | 5541 | 8.90% |
| 17 | 18.569 | 4.7745 | 29284 | 30379 | 58.80% |
| 18 | 18.965 | 4.67564 | 888 | 1961 | 1.80% |
| 19 | 19.962 | 4.44445 | 2898 | 3937 | 5.80% |
| 20 | 20.329 | 4.36498 | 870 | 1910 | 1.70% |
| 21 | 20.922 | 4.24243 | 4014 | 5041 | 8.10% |
| 22 | 21.245 | 4.17879 | 1214 | 2225 | 2.40% |
| 23 | 21.72 | 4.0884 | 1929 | 2907 | 3.90% |
| 24 | 22.227 | 3.99629 | 4046 | 4975 | 8.10% |
| 25 | 23.076 | 3.85122 | 3967 | 4821 | 8.00% |
| 26 | 23.362 | 3.80459 | 465 | 1301 | 0.90% |
| 27 | 23.942 | 3.71371 | 512 | 1297 | 1.00% |
| 28 | 24.805 | 3.58646 | 1852 | 2588 | 3.70% |
| 29 | 25.498 | 3.49057 | 267 | 964 | 0.50% |
| 30 | 25.817 | 3.44821 | 352 | 1031 | 0.70% |
| 31 | 26.017 | 3.42212 | 387 | 1053 | 0.80% |
| 32 | 26.711 | 3.33473 | 170 | 799 | 0.30% |
| 33 | 27.337 | 3.25982 | 209 | 843 | 0.40% |
| 34 | 27.528 | 3.2376 | 636 | 1267 | 1.30% |
| 35 | 28.157 | 3.16664 | 70.6 | 707 | 0.10% |
| 36 | 28.504 | 3.1289 | 702 | 1355 | 1.40% |
| 37 | 28.895 | 3.08749 | 380 | 1045 | 0.80% |
| 38 | 29.52 | 3.02352 | 840 | 1505 | 1.70% |
| 39 | 30.458 | 2.9325 | 771 | 1399 | 1.50% |
| 40 | 31.135 | 2.87021 | 144 | 758 | 0.30% |
| 41 | 31.674 | 2.82262 | 606 | 1230 | 1.20% |
| 42 | 32.376 | 2.763 | 143 | 760 | 0.30% |
| 43 | 32.829 | 2.72595 | 252 | 855 | 0.50% |
| 44 | 33.26 | 2.69157 | 81.9 | 661 | 0.20% |
| 45 | 33.746 | 2.65388 | 79 | 652 | 0.20% |
| 46 | 34.479 | 2.59911 | 410 | 988 | 0.80% |
| 47 | 34.856 | 2.57192 | 126 | 695 | 0.30% |
| 48 | 36.397 | 2.46643 | 946 | 1536 | 1.90% |
| 49 | 36.297 | 2.47302 | 315 | 900 | 0.60% |
| 50 | 36.397 | 2.46645 | 946 | 1535 | 1.90% |
| 51 | 36.873 | 2.43568 | 484 | 1090 | 1.00% |
| 52 | 37.502 | 2.39628 | 286 | 895 | 0.60% |
| 53 | 37.601 | 2.39023 | 143 | 752 | 0.30% |
| 54 | 38.553 | 2.33332 | 100 | 692 | 0.20% |
| 55 | 38.923 | 2.31198 | 187 | 775 | 0.40% |
| 56 | 40.424 | 2.22956 | 79.7 | 691 | 0.20% |
| 57 | 40.631 | 2.21868 | 163 | 788 | 0.30% |
| 58 | 41.445 | 2.17698 | 126 | 777 | 0.30% |
| 59 | 41.724 | 2.16302 | 355 | 1008 | 0.70% |
| 60 | 42.97 | 2.10315 | 292 | 922 | 0.60% |
| 61 | 43.865 | 2.06231 | 527 | 1125 | 1.10% |

FIG. 6X is an XRPD of solid polymorph A obtained by crystallization from an isopropyl alcohol/water solvent/anti-solvent medium in a ratio of 1:1 v/v at a precipitating temperature of 20° C. FIG. 6W is an XRPD of a solid OXY133 Form A. Table 7, below lists data taken from the XRPD of FIG. 6X. As illustrated in Table 7, OXY133 Form A can have one or more reflections of different relative intensities at index numbers 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 and 70.

TABLE 7
XRPD Data for OXY133 Form A, as illustrated in FIG. 6X

| | | | Net | Gross | Rel. |
|---|---|---|---|---|---|
| | Angle | d Value | Intensity | Intensity | Intensity |
| Index No. | (2-Theta) | (Angstrom) | (Counts) | (Counts) | (%) |
| 0 | 6.098 | 14.48166 | 71894 | 72673 | 55.80% |
| 1 | 7.555 | 11.69167 | 620 | 1477 | 0.50% |
| 2 | 10.796 | 8.18802 | 304 | 1200 | 0.20% |
| 3 | 10.984 | 8.04836 | 9113 | 10057 | 7.10% |
| 4 | 12.071 | 7.3264 | 5328 | 6503 | 4.10% |
| 5 | 12.304 | 7.18792 | 128932 | 130146 | 100.00% |
| 6 | 13.144 | 6.73047 | 5158 | 6472 | 4.00% |
| 7 | 14.037 | 6.30435 | 4918 | 6268 | 3.80% |
| 8 | 14.399 | 6.14643 | 282 | 1626 | 0.20% |
| 9 | 14.654 | 6.04022 | 1001 | 2335 | 0.80% |
| 10 | 15.26 | 5.8014 | 170 | 1454 | 0.10% |
| 11 | 16.1 | 5.50083 | 2296 | 3687 | 1.80% |
| 12 | 16.397 | 5.40158 | 43971 | 45425 | 34.10% |
| 13 | 16.695 | 5.30599 | 10019 | 11529 | 7.80% |
| 14 | 17.36 | 5.10419 | 8778 | 10385 | 6.80% |
| 15 | 17.632 | 5.02597 | 2526 | 4161 | 2.00% |
| 16 | 17.91 | 4.94862 | 14487 | 16144 | 11.20% |
| 17 | 18.585 | 4.77037 | 83433 | 85113 | 64.70% |
| 18 | 19.032 | 4.65948 | 1275 | 2949 | 1.00% |
| 19 | 19.934 | 4.45053 | 8143 | 9749 | 6.30% |
| 20 | 20.456 | 4.33802 | 646 | 2180 | 0.50% |
| 21 | 20.94 | 4.23896 | 10923 | 12389 | 8.50% |
| 22 | 21.236 | 4.18054 | 1039 | 2460 | 0.80% |
| 23 | 21.561 | 4.11829 | 1073 | 2437 | 0.80% |
| 24 | 21.999 | 4.03729 | 606 | 1877 | 0.50% |
| 25 | 22.241 | 3.9938 | 4339 | 5551 | 3.40% |
| 26 | 23.036 | 3.85779 | 1695 | 2784 | 1.30% |
| 27 | 23.335 | 3.80903 | 1188 | 2257 | 0.90% |
| 28 | 23.946 | 3.71319 | 759 | 1760 | 0.60% |
| 29 | 24.355 | 3.6518 | 355 | 1319 | 0.30% |
| 30 | 24.849 | 3.58023 | 3968 | 4914 | 3.10% |
| 31 | 25.493 | 3.49128 | 258 | 1156 | 0.20% |
| 32 | 25.8 | 3.45038 | 751 | 1620 | 0.60% |
| 33 | 26.09 | 3.4127 | 1079 | 1914 | 0.80% |
| 34 | 26.697 | 3.33648 | 105 | 924 | 0.10% |
| 35 | 27.048 | 3.29392 | 1090 | 1924 | 0.80% |
| 36 | 27.552 | 3.2348 | 1009 | 1847 | 0.80% |
| 37 | 28.06 | 3.1774 | 74.6 | 925 | 0.10% |
| 38 | 28.531 | 3.12604 | 996 | 1864 | 0.80% |
| 39 | 28.853 | 3.09189 | 297 | 1166 | 0.20% |
| 40 | 29.497 | 3.0258 | 1608 | 2463 | 1.20% |
| 41 | 30.333 | 2.94428 | 327 | 1131 | 0.30% |
| 42 | 30.446 | 2.93364 | 211 | 1004 | 0.20% |
| 43 | 31.115 | 2.87207 | 406 | 1179 | 0.30% |
| 44 | 31.337 | 2.85218 | 369 | 1156 | 0.30% |
| 45 | 31.723 | 2.81839 | 1675 | 2476 | 1.30% |
| 46 | 32.331 | 2.76679 | 224 | 1025 | 0.20% |
| 47 | 32.867 | 2.72284 | 650 | 1431 | 0.50% |
| 48 | 33.302 | 2.68829 | 188 | 934 | 0.10% |
| 49 | 33.694 | 2.65791 | 212 | 939 | 0.20% |
| 50 | 33.908 | 2.6416 | 162 | 893 | 0.10% |
| 51 | 34.422 | 2.60328 | 260 | 982 | 0.20% |
| 52 | 34.531 | 2.59532 | 583 | 1300 | 0.50% |
| 53 | 34.679 | 2.58463 | 109 | 818 | 0.10% |
| 54 | 35.32 | 2.53919 | 105 | 773 | 0.10% |
| 55 | 35.938 | 2.49693 | 85.7 | 764 | 0.10% |
| 56 | 36.216 | 2.47835 | 658 | 1373 | 0.50% |
| 57 | 37.079 | 2.42262 | 294 | 1076 | 0.20% |
| 58 | 37.698 | 2.3843 | 685 | 1473 | 0.50% |
| 59 | 37.996 | 2.36626 | 158 | 938 | 0.10% |
| 60 | 38.486 | 2.33725 | 106 | 862 | 0.10% |
| 61 | 38.869 | 2.31508 | 312 | 1041 | 0.20% |
| 62 | 39.06 | 2.30421 | 304 | 1014 | 0.20% |
| 63 | 40.087 | 2.24749 | 111 | 827 | 0.10% |
| 64 | 40.668 | 2.21676 | 580 | 1364 | 0.40% |
| 65 | 41.302 | 2.18417 | 164 | 988 | 0.10% |
| 66 | 41.677 | 2.16539 | 246 | 1076 | 0.20% |
| 67 | 42.01 | 2.14897 | 339 | 1165 | 0.30% |
| 68 | 42.405 | 2.12988 | 133 | 941 | 0.10% |
| 69 | 42.824 | 2.10999 | 705 | 1478 | 0.50% |
| 70 | 43.206 | 2.09221 | 126 | 853 | 0.10% |

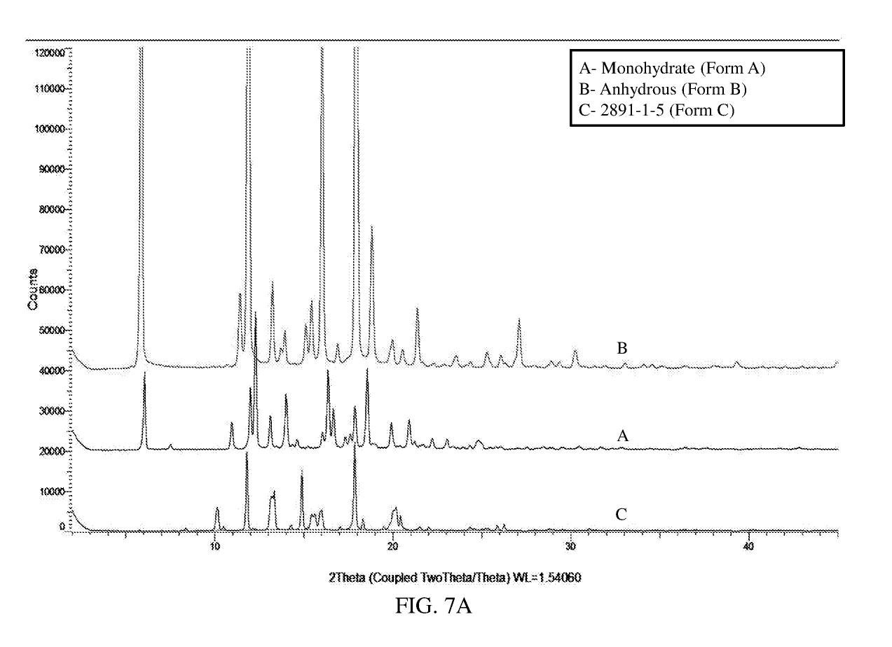

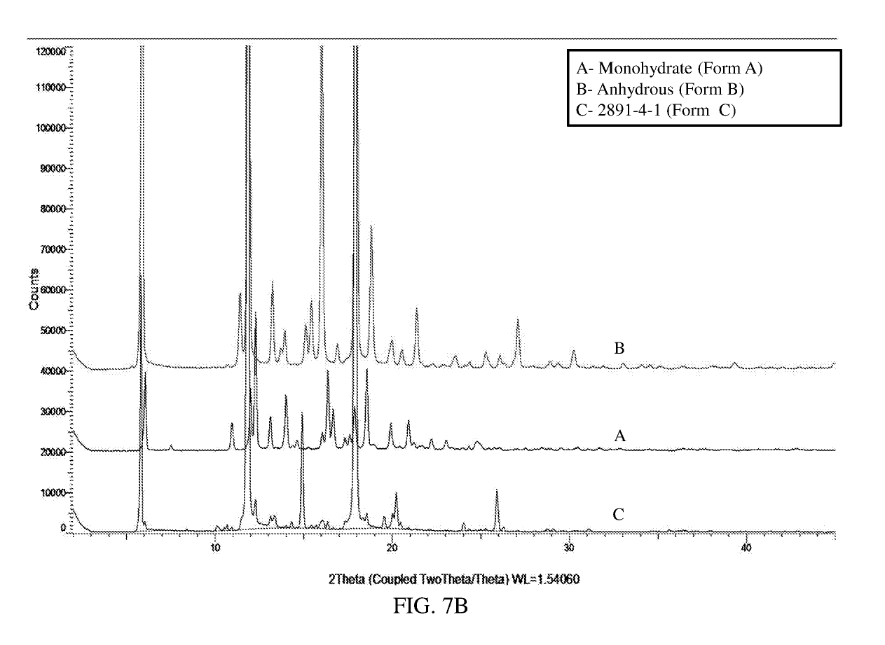

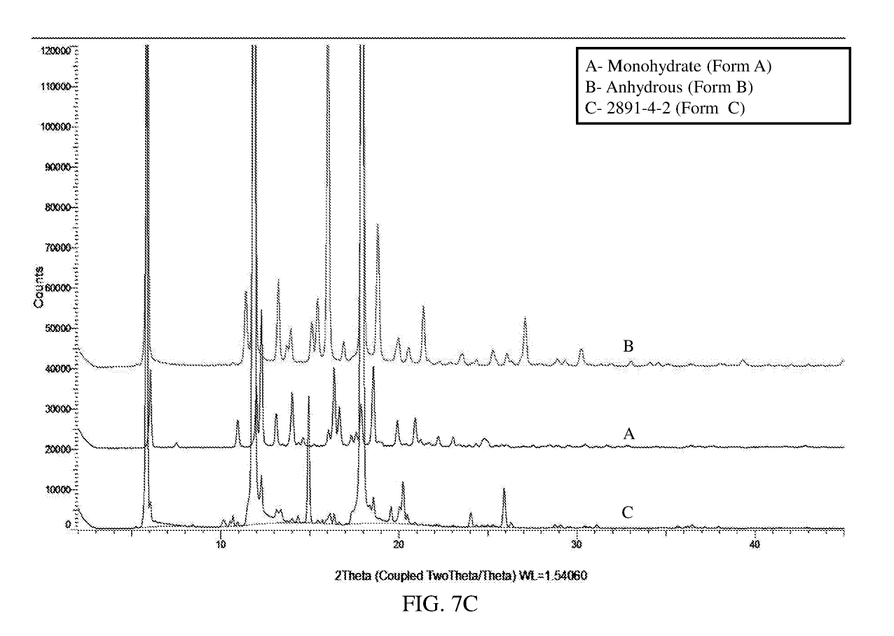

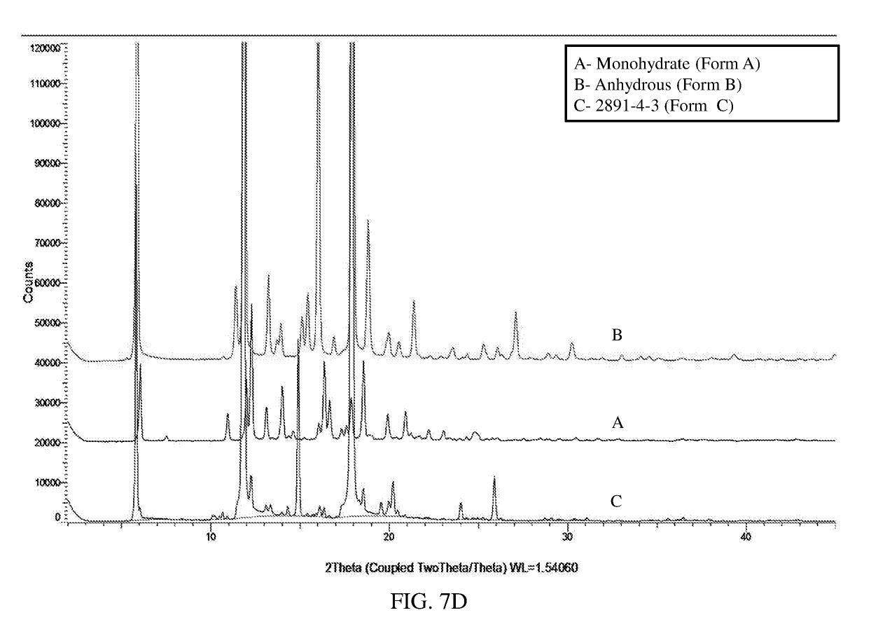

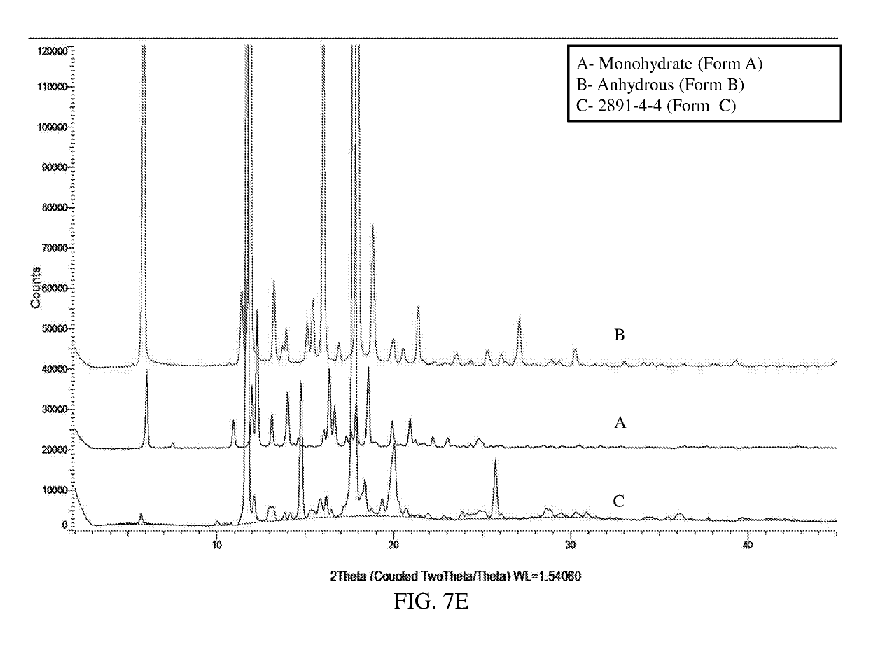

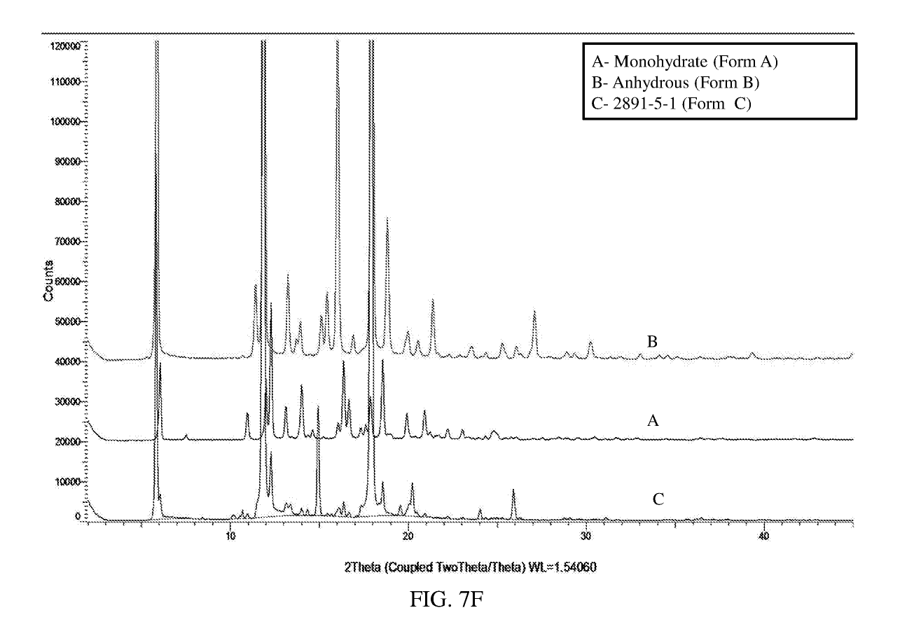

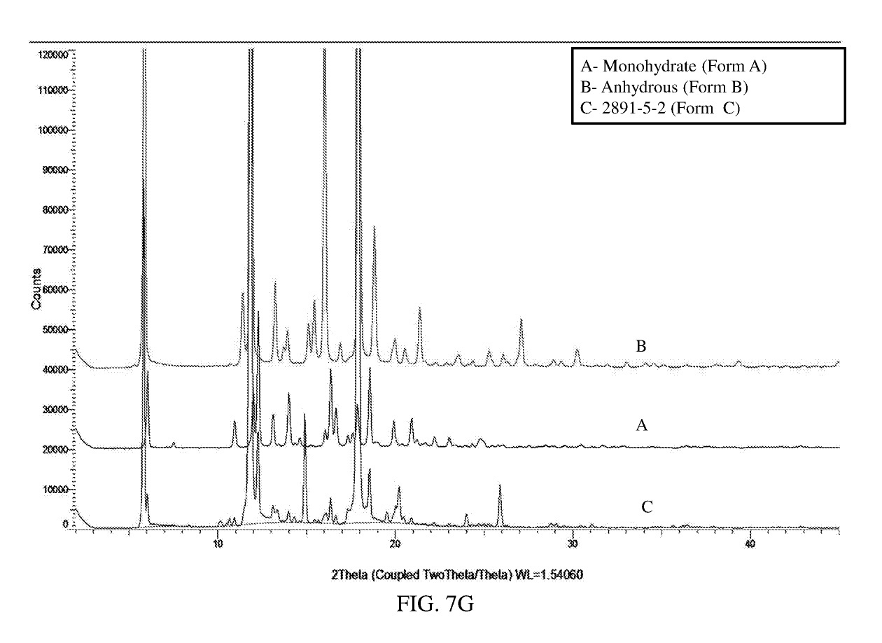

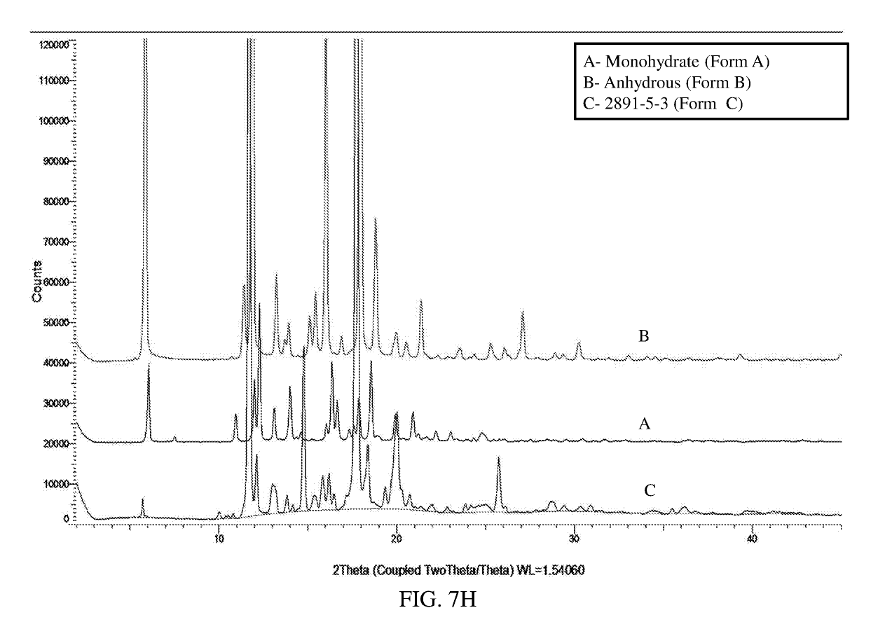

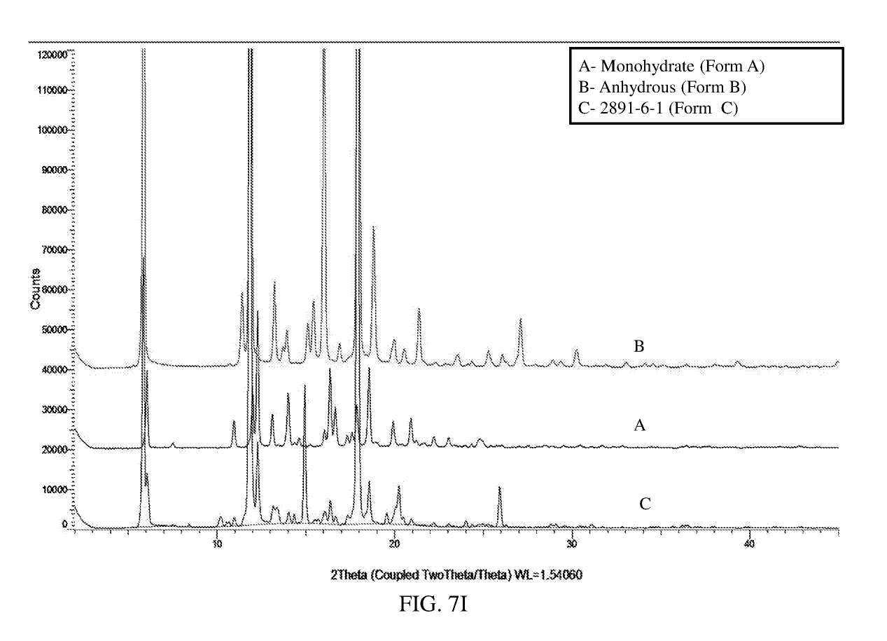

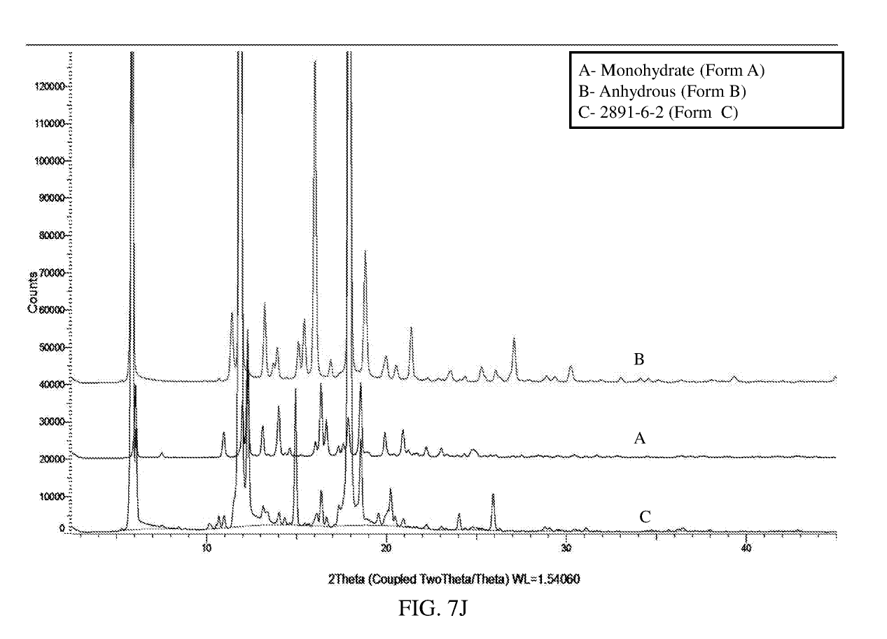

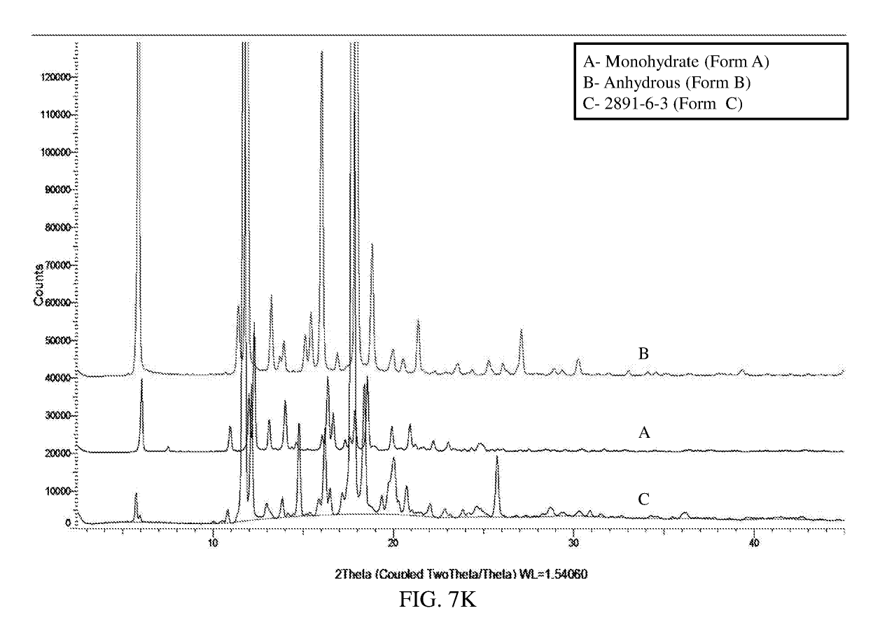

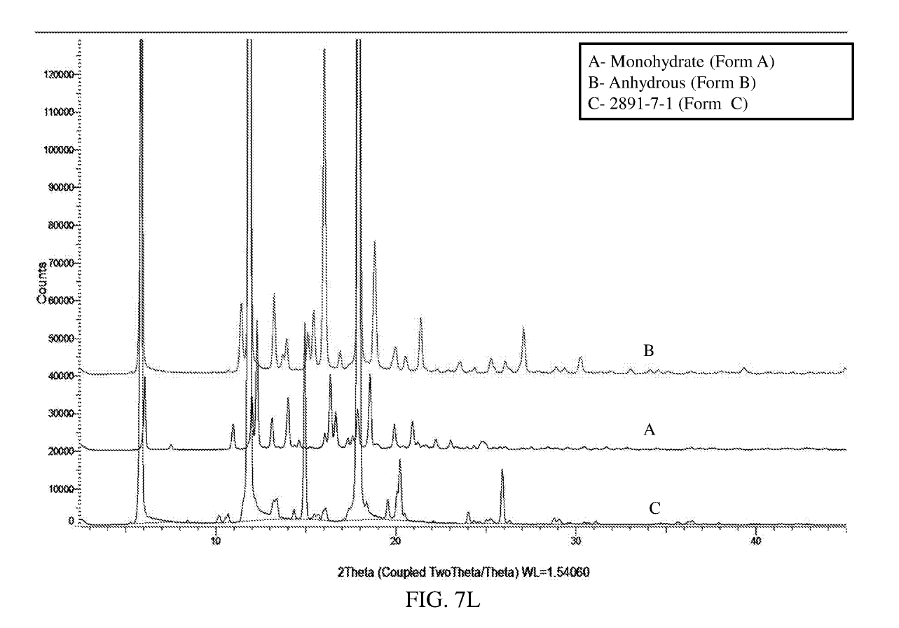

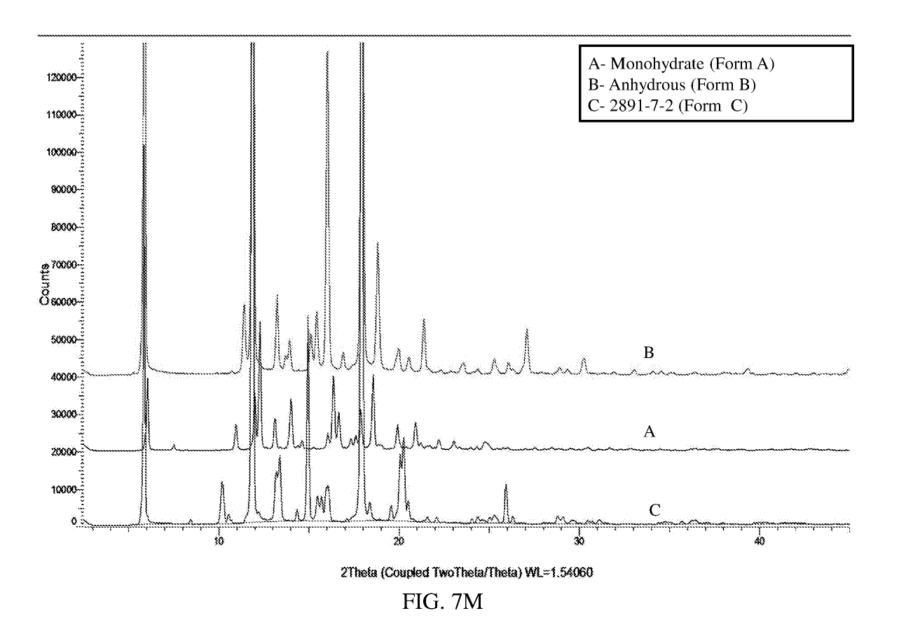

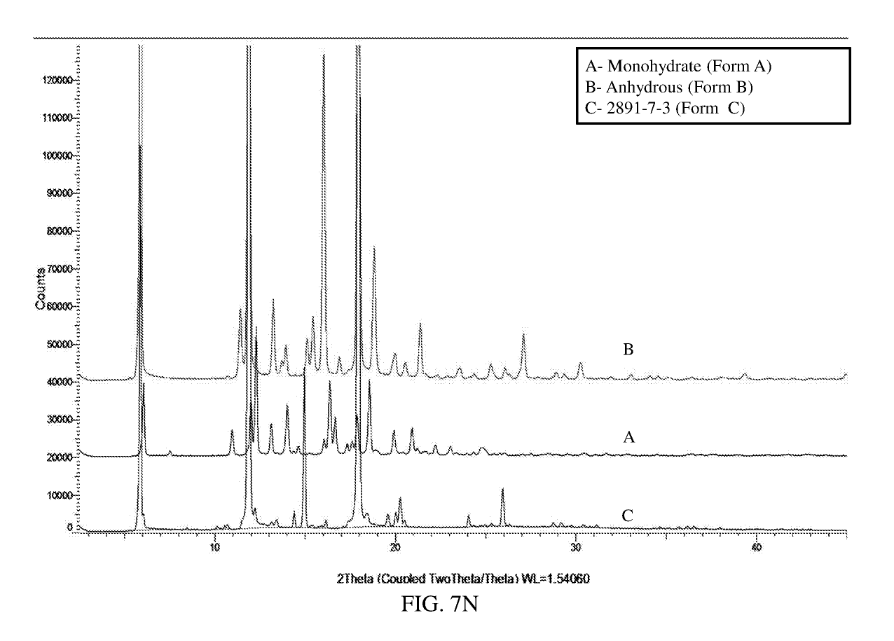

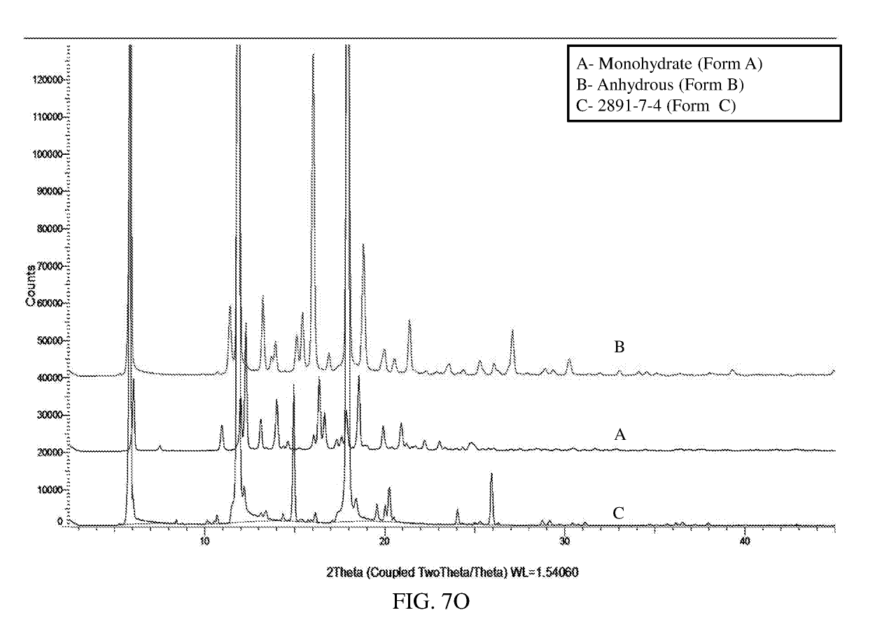

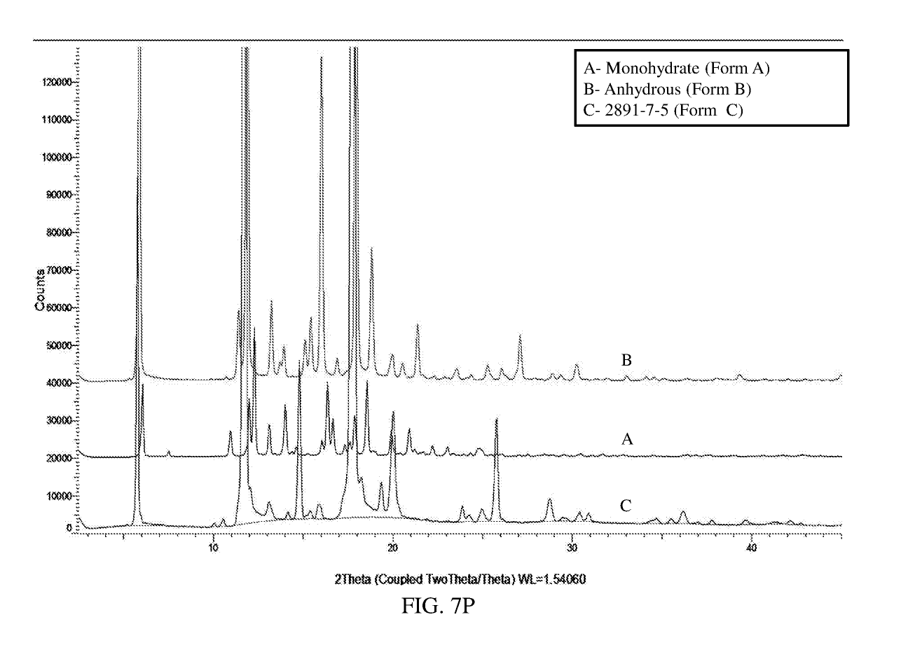

FIGS. 7A, 7B, 7C, 7D, 7E, 7F, 7G, 7H, 7I, 7J, 7K, 7L, 7M, 7N, 70, and 7P are XRPDs of OXY133 polymorph Form C. In particular, these figures are XRPDs of polymorph Form C obtained by re-slurrying from an acetone/water solvent system, where the acetone is the solvent and the water is the anti-solvent, in a ratio of 1:1 v/v. The precipitating temperatures at which these XRPDs were obtained are listed in more detail in Table 4 below and include 10° C., 20° C., 30° C., 40° C., 50° C., 60° C. and 70° C., respectively.

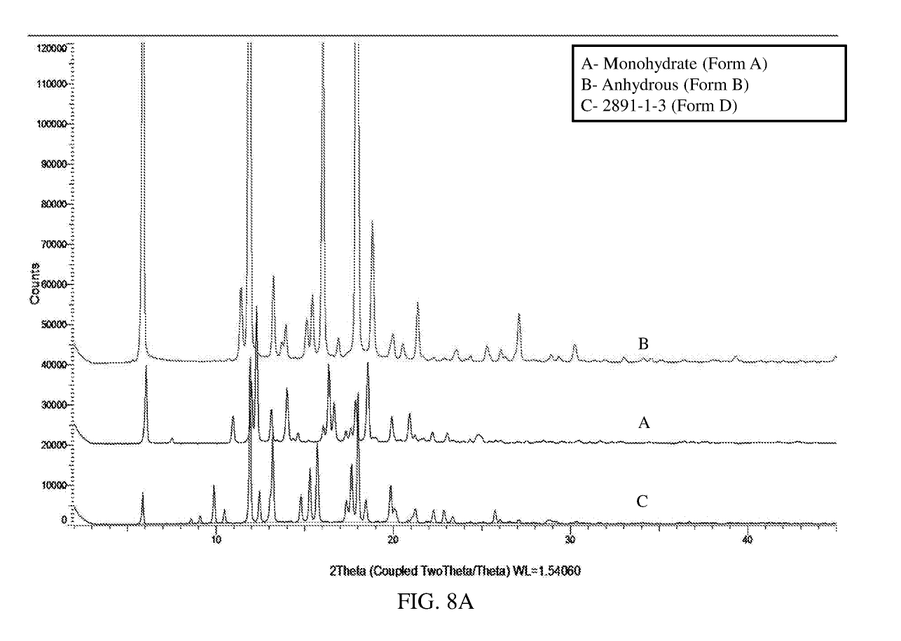

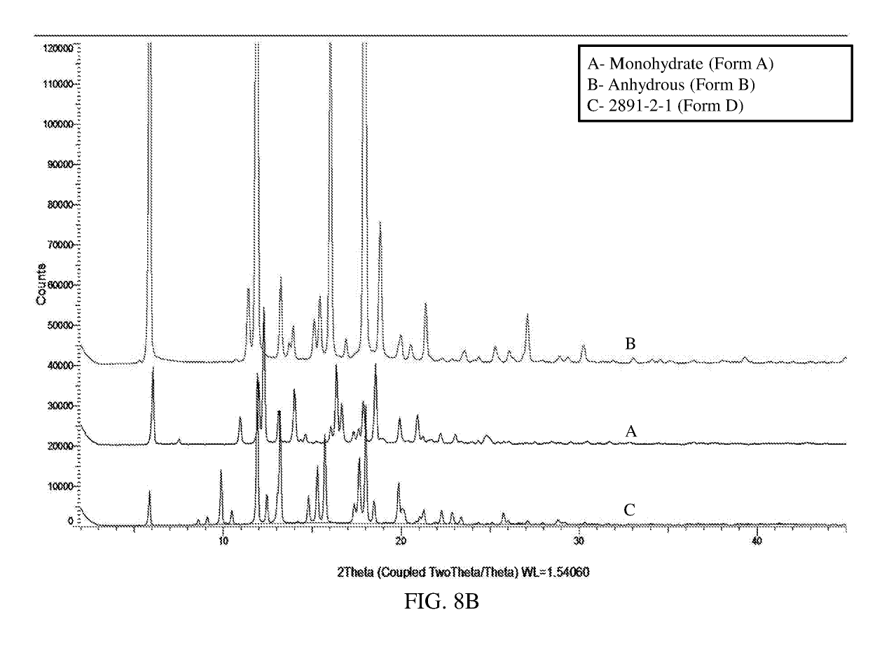

FIGS. 8A and 8B are XRPDs of OXY133 polymorph Form D obtained by re-slurrying from a methanol/water system at a precipitating temperature of 20° C. and 70° C., respectively.

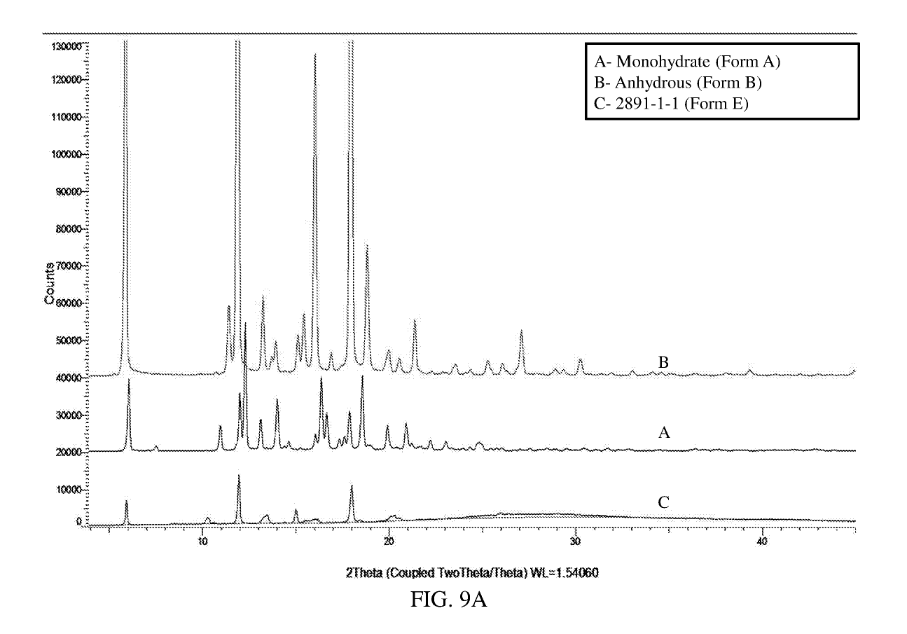

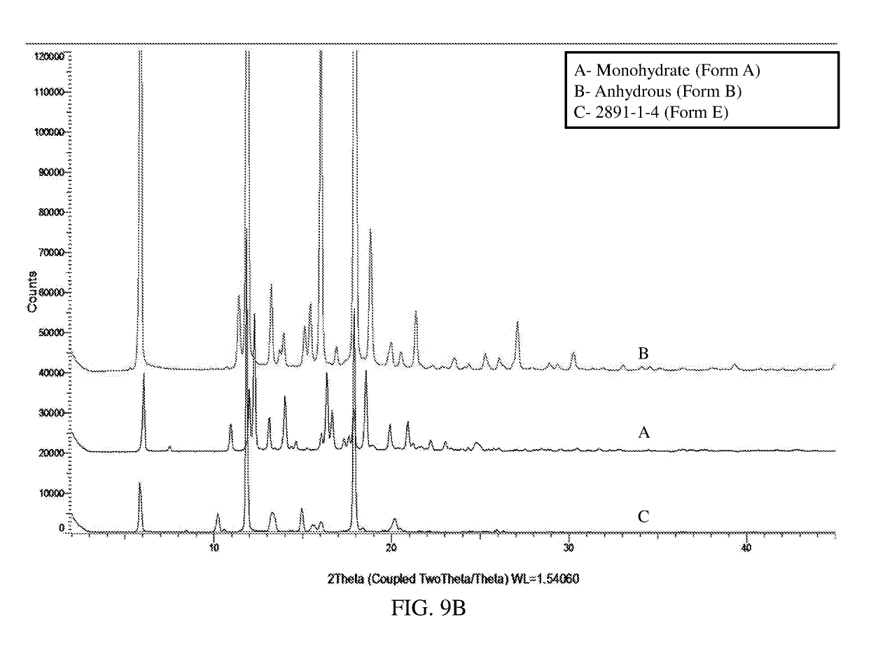

FIGS. 9A and 9B are XRPDs of OXY133 polymorph Form E obtained from a slurry of OXY133 polymorph Form B in water at temperatures of 20° C. and 70° C., respectively.

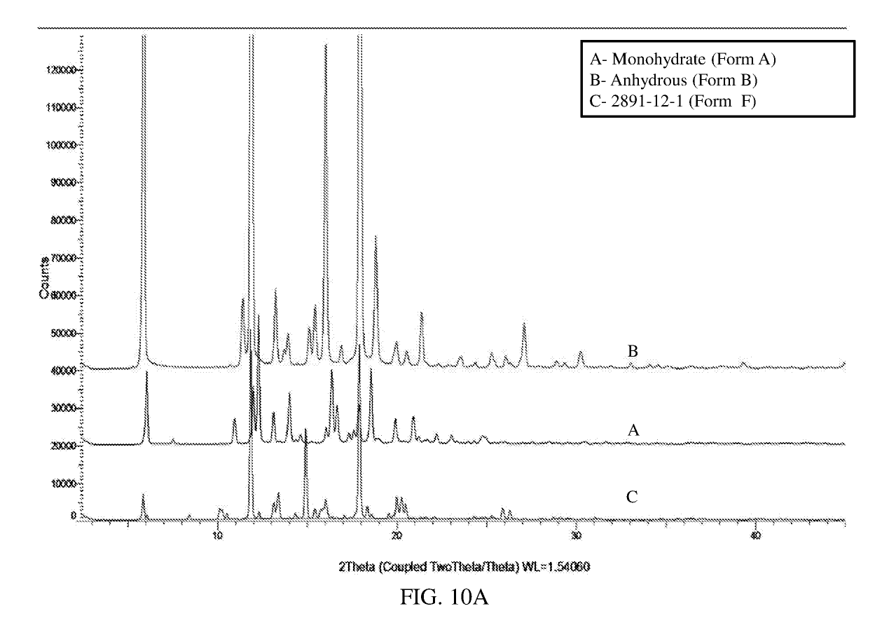

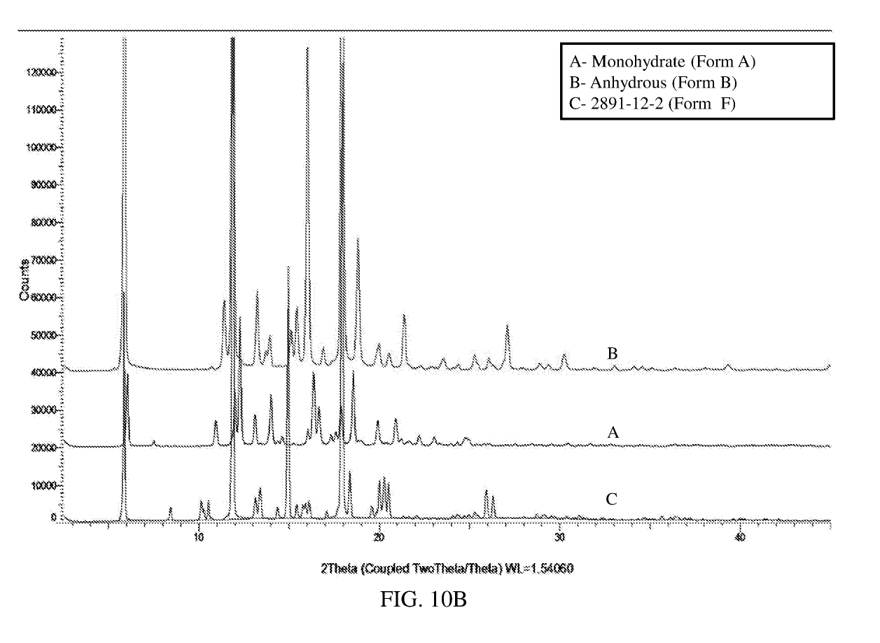

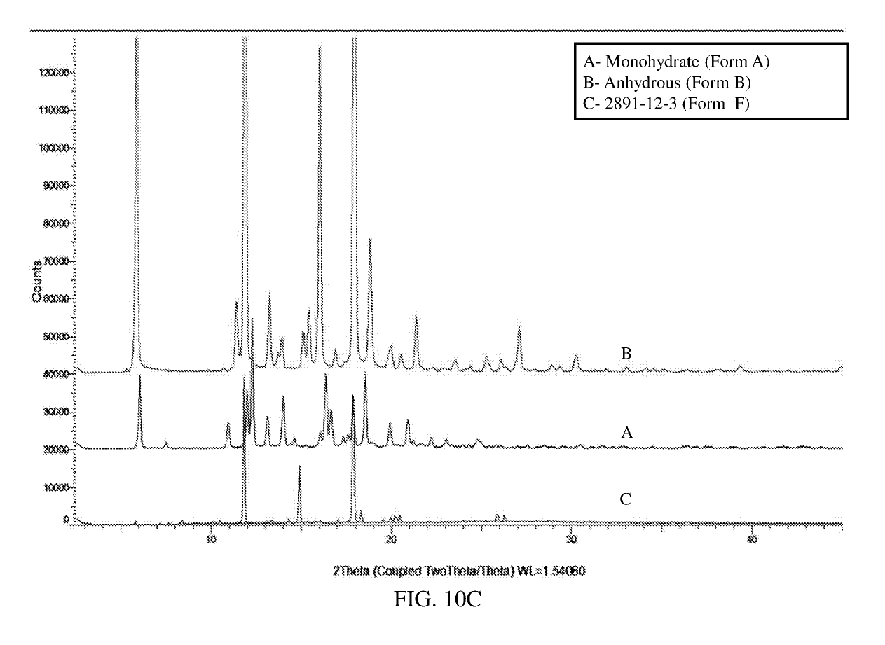

FIGS. 10A, 10B, and 10C are XRPDs of OXY133 polymorph Form F obtained by a dissolution in acetone/water followed by precipitation at temperatures of 5° C. and 15° C., respectively.

FIG. 11 is an XRPD of OXY133 polymorph Form G obtained by crystallization from IPA/water solvent system in a ratio of 1:2 v/v at a temperature of 40° C.

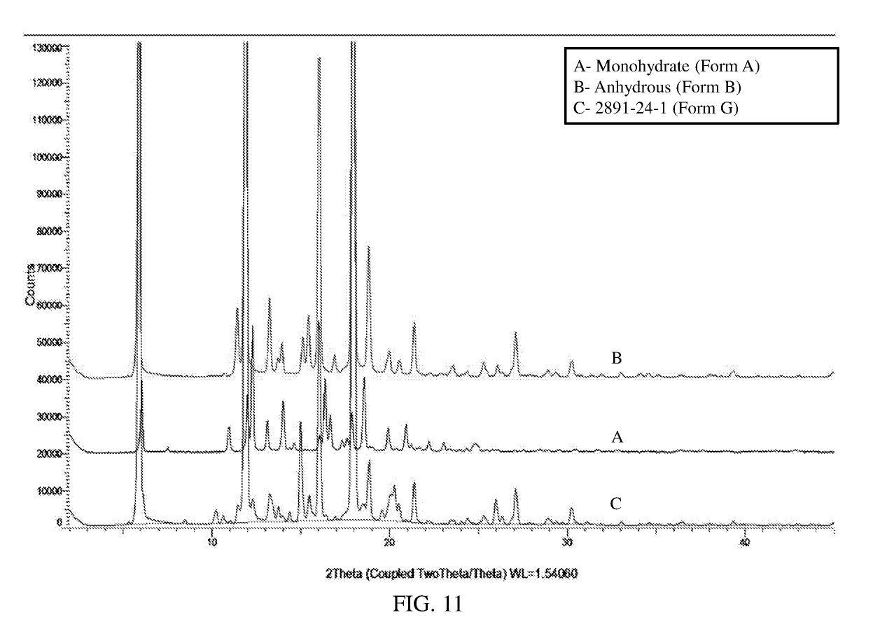

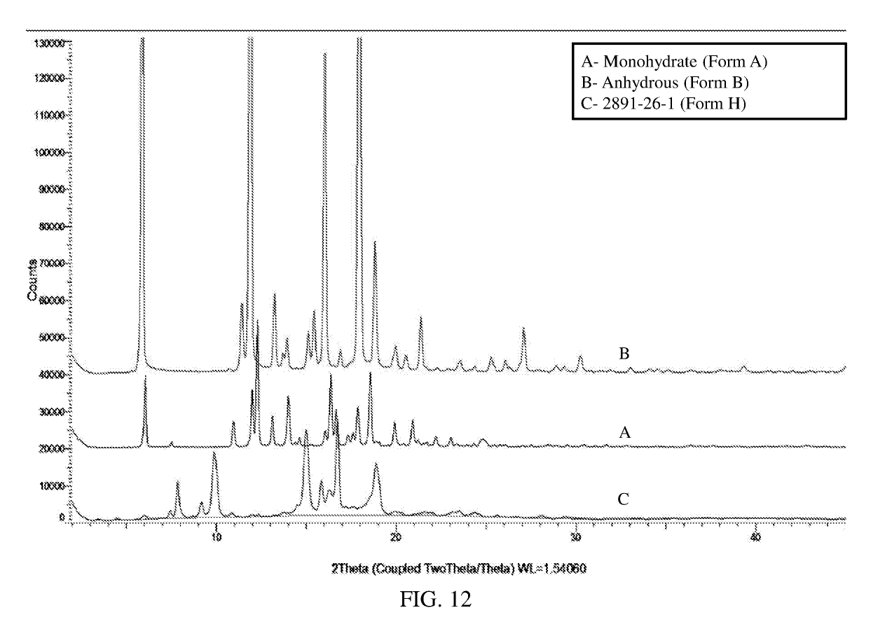

FIG. 12 is an XRPD of OXY133 polymorph Form H obtained by crystallization from IPA/water solvent system in a ratio of 1:2 v/v at a temperature of −10° C.

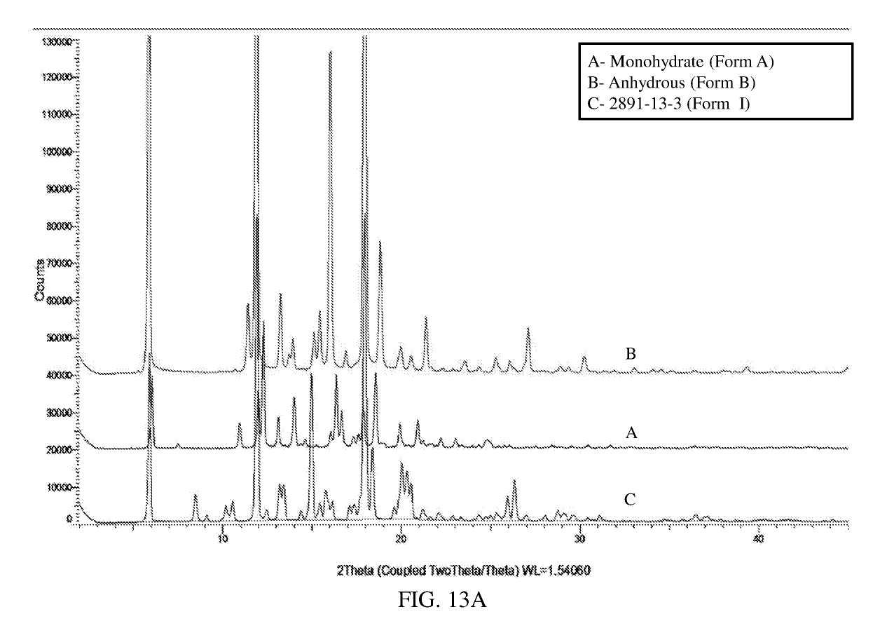

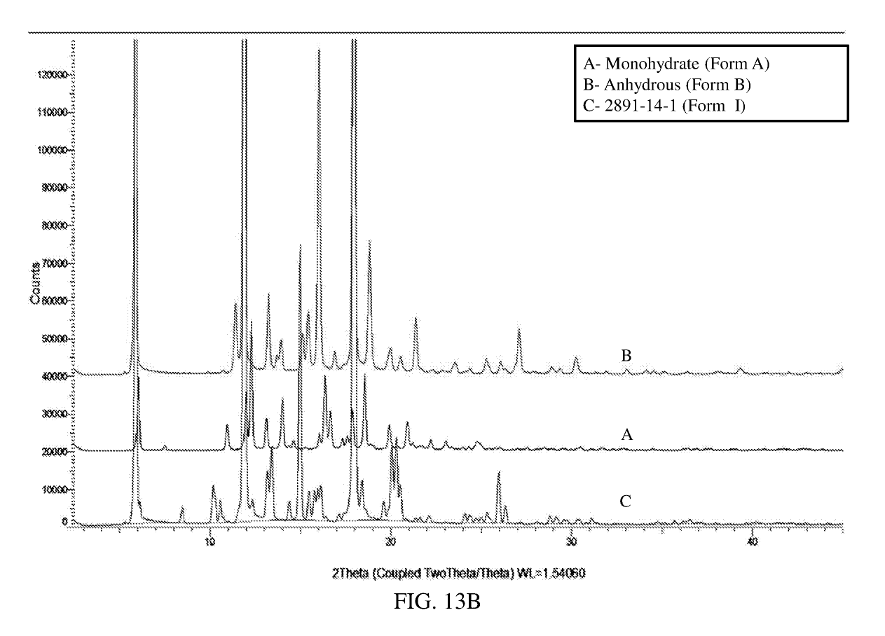

FIGS. 13A and 13B are XRPDs of OXY133 polymorph Form I formed by re-slurrying from a methanol/acetone/water solvent system or by recrystallization from acetone at 20° C., respectively.

The equipment utilized to collect the XRPD patterns depicted in FIGS. 2A-13B was a Bruker D8 Advance diffractometer using Cu radiation (40 kV, 25 mA) with a divergence slit of 0.3° (0.6 mm), wherein variable slits must be operated in fixed mode. The axial Soller slits, primary and secondary were each set at 2.5°. The anti-scatter slit was set at 0.3° (0.6 mm). The secondary monochromator anti-scatter slit was set at 1 mm and the detector slit at 0.1 mm. If a secondary monochromator is not used, then a suitable 3 filter must be used, namely a Ni filter for Cu radiation. The linear detector LYNXEYE was set at 30 detector opening with the angle scanned from 2 to 45° 2Θ.

Further, Table 8 below is a list of OXY133 polymorphs Forms A, B, C, D, E, F, G, H and I identified by a high performance liquid chromatography (HPLC) followed by charged aerosol detector (CAD) method. Table 4 also lists the starting products, the solvent system including solvent and anti-solvent, the temperature at which a polymorph was formed, the water content of the polymorph as determined by the Karl-Fisher (KF) method of water determination, and where available the yield and purity of the resulting polymorph.

TABLE 8
HPLC Method: OXY133 Polymorphs (CAD)

| | | | | | | | | | | Crystal | | |
|---|---|---|---|---|---|---|---|---|---|---|---|---|
| | | | Processing | Temp | | | time | | TGA | Form | Yield | Purity |
| Item | Method | Sample | Point | (C.) | Scale | | (h) | KF | (% LOD) | (XRPD | (%) | (%) |
| 1 | OXY133 Anhydrous | 55352-23-07 | Solid | 20 | NA | | NA | NA | 0.34 | Form B | NA | 96.88 |
| | From Medtronic | | | | | | | | | | | |
| 2 | OXY133 | 82489-2-7-1 | Solid | 20 | NA | | NA | 4.1 | 5.28 | Form A | NA | NA |
| | Monohydrate From | | | | | | | | | | | |
| | Medtronic | | | | | | | | | | | |
| 3 | Slurry of Form B in | 2891-1-1 | Slurry | 20 | 24.6 | mg | 24 | NA | NA | Form E | NA | NA |
| | Water at 20° C. | | | | | | | | | | | |
| 4 | Slurry of Form B in | 2891-1-4 | Slurry | 70 | 19.6 | mg | 24 | NA | NA | Form E | NA | NA |
| | Water at 70° C. | | | | | | | | | | | |
| 5 | Acetone/Water | 2891-1-2 | Slurry | 20 | 28.0 | mg | 24 | NA | NA | Form A | NA | NA |
| | (1:1) at 20° C. | | | | | | | | | | | |
| 6 | Acetone/Water | 2891-1-5 | Slurry | 70 | 19.4 | mg | 24 | NA | NA | Form C | NA | NA |
| | (1:1) at 70° C. | | | | | | | | | | | |
| 7 | MeOH/Water | 2891-1-3 | Slurry | 20 | 22.5 | mg | 24 | NA | NA | Form D | NA | NA |
| | (1:1) at 20° C. | | | | | | | | | | | |
| 8 | MeOH/Water | 2891-2-1 | Slurry | 70 | 22.0 | mg | 24 | NA | NA | Form D | NA | NA |
| | (1:1) at 70° C. | | | | | | | | | | | |
| 9 | Acetone/Water (1:1) | 2891-3-1 | Slurry | 20 | 2.0 | g | 22 | NA | NA | Form A + | NA | NA |
| | Overhead stirring | | | | | | | | | Form B | | |
| 10 | Acetone/Water (1:1) | 2891-3-2 | Slurry | 20 | 2.0 | g | 51 | NA | NA | Form A | NA | NA |
| | Overhead stirring | | | | | | | | | | | |
| 11 | Acetone/Water (1:1) | 2891-3-4 | Solid after | 50 | 2.0 | g | NA | NA | 4.1 | Form A | 75.0 | NA |
| | Overhead stirring | | drying at | | | | | | | | | |
| | | | 50° C. | | | | | | | | | |
| 12 | Acetone/Water | 2891-4-1 | Slurry | 30 | 2.0 | g | 3 | NA | NA | Form C | NA | NA |
| | (1:1) 30° C. | | | | | | | | | | | |
| 13 | Acetone/Water | 2891-4-2 | Slurry | 30 | 2.0 | g | 23 | NA | NA | Form C | NA | NA |
| | (1:1) 30° C. | | | | | | | | | | | |
| 14 | Acetone/Water | 2891-4-3 | Slurry | 30 | 2.0 | g | 47 | NA | NA | Form C | NA | NA |
| | (1:1) 30° C. | | | | | | | | | | | |
| 15 | Acetone/Water | 2891-4-4 | Solid after | 30 | 2.0 | g | NA | NA | NA | Form C | 78.9 | NA |
| | (1:1) 30° C. | | drying at | | | | | | | | | |
| | | | 35° C. | | | | | | | | | |
| 16 | Acetone/Water | 2891-5-1 | Slurry | 40 | 2.0 | g | 3 | NA | NA | Form C | NA | NA |
| | (1:1) 40° C. | | | | | | | | | | | |
| 17 | Acetone/Water | 2891-5-2 | Slurry | 40 | 2.0 | g | 23 | NA | NA | Form C | NA | NA |
| | (1:1) 40° C. | | | | | | | | | | | |
| 18 | Acetone/Water | 2891-5-3 | Solid after | 40 | 2.0 | g | NA | NA | NA | Form C | 80.9 | NA |
| | (1:1) 40° C. | | drying at | | | | | | | | | |
| | | | 35° C. | | | | | | | | | |
| 19 | Acetone/Water | 2891-6-1 | Slurry | 50 | 2.0 | g | 3 | NA | NA | Form C | NA | NA |
| | (1:1) 50° C. | | | | | | | | | | | |
| 20 | Acetone/Water | 2891-6-2 | Slurry | 50 | 2.0 | g | 23 | NA | NA | Form C | NA | NA |
| | (1:1) 50° C. | | | | | | | | | | | |
| 21 | Acetone/Water | 2891-6-3 | Solid after | 50 | 2.0 | g | NA | NA | NA | Form C | 84.7 | NA |
| | (1:1) 50° C. | | drying at | | | | | | | | | |
| | | | 35° C. | | | | | | | | | |
| 22 | Acetone/Water | 2891-7-1 | Slurry | 60 | 2.0 | g | 3 | NA | NA | Form C | NA | NA |
| | (1:1) 60° C. | | | | | | | | | | | |
| 23 | Acetone/Water | 2891-7-2 | Slurry | 60 | 2.0 | g | 23 | NA | NA | Form C | NA | NA |
| | (1:1) 60° C. | | | | | | | | | | | |
| 24 | Acetone/Water | 2891-7-3 | Slurry | 10 | 2.0 | g | 43 | NA | NA | Form C | NA | NA |
| | (1.1) 60° C. | | | | | | | | | | | |
| 25 | Acetone/Water | 2891-7-4 | Slurry | 20 | 2.0 | g | 65 | NA | 1.9 | Form C | NA | NA |
| | (1.1) 60° C. | | | | | | | | | | | |
| 26 | Acetone/Water | 2891-7-5 | Solid after | 20 | 2.0 | g | NA | 1.45 | NA | Form C | 84.2 | NA |
| | (1:1) 60° C. | | drying at | | | | | | | | | |
| | | | 35° C. | | | | | | | | | |
| 27 | Acetone/Water | 2891-8-1 | Slurry | 0 | 2.0 | g | 20 | NA | NA | Form A + | NA | NA |
| | (1:1) 0° C. | | | | | | | | | Form B | | |
| 28 | Acetone/Water | 2891-8-2 | Slurry | 0 | 2.0 | g | 46 | NA | NA | Form A | NA | NA |
| | (1:1) 0° C. | | | | | | | | | | | |
| 29 | Acetone/Water | 2891-8-3 | Solid after | 35 | 2.0 | g | NA | 3.25 | NA | Form A | 89.5 | 96.79 |
| | (1:1) 0° C. | | drying at | | | | | | | | | |
| | | | 35° C. | | | | | | | | | |
| 30 | Acetone/Water | 2891-9-1 | Slurry | 10 | 2.0 | g | 20 | NA | NA | Form A + | NA | NA |
| | (1:1) 10° C. | | | | | | | | | Form B | | |
| 31 | Acetone/Water | 2891-9-2 | Slurry | 10 | 2.0 | g | 46 | NA | NA | Form A | NA | NA |
| | (1:1) 10° C. | | | | | | | | | | | |
| 32 | Acetone/Water | 2891-9-3 | Solid after | 35 | 2.0 | g | NA | 3.83 | NA | Form A | 99.0 | 96.75 |
| | (1:1) 10° C. | | drying at | | | | | | | | | |
| | | | 35° C. | | | | | | | | | |
| 33 | Acetone/Water | 2891-10-1 | Slurry | 25 | 2.0 | g | 20 | NA | NA | Form A + | NA | NA |
| | (1:1) 25° C. | | | | | | | | | Form B | | |
| 34 | Acetone/Water | 2891-10-2 | Slurry | 25 | 2.0 | g | 46 | NA | NA | Form A | NA | NA |
| | (1:1) 25° C. | | | | | | | | | | | |
| 35 | Acetone/Water | 2891-10-3 | Solid after | 35 | 2.0 | g | NA | 4.02 | NA | Form A | 82.6 | 96.97 |
| | (1:1) 25° C. | | drying at | | | | | | | | | |
| | | | 35° C. | | | | | | | | | |
| 36 | Acetone/Water | 2891-12-1 | Slurry after | 15 | 4.0 | g | 0 | NA | NA | Form F | NA | NA |
| | Dissolution/ | | charging | | | | | | | | | |
| | Precipitation | | water | | | | | | | | | |
| 37 | Acetone/Water | 2891-12-2 | Slurry | 5 | 4.0 | g | 20 | NA | NA | Form F | NA | NA |
| | Dissolution/ | | | | | | | | | | | |
| | Precipitation | | | | | | | | | | | |
| 38 | Acetone/Water | 2891-12-3 | Slurry | 5 | 4.0 | g | 45 | NA | NA | Form F | NA | NA |
| | Dissolution/ | | | | | | | | | | | |
| | Precipitation | | | | | | | | | | | |
| 39 | Acetone | 2891-14-1 | Solid | 20 | 2.0 | g | 1 | NA | NA | Form I | NA | 98.87 |
| | Recrystallization | | | | | | | | | | | |
| 40 | THF/Acetone/ | 2891-16-1 | Solid before | 35 | 4.0 | g | NA | NA | NA | Form A | NA | NA |
| | Water | | drying | | | | | | | | | |
| | Crystallization | | | | | | | | | | | |
| 41 | THF/Acetone/ | 2891-16-2 | Solid after | 35 | 4.0 | g | NA | 3.37 | NA | Form A | 60.0 | 99.31 |
| | Water | | drying at | | | | | | | | | |
| | Crystallization | | 35° C. | | | | | | | | | |
| 42 | THF/Water (1:2) | 2891-17-1 | Oil/Slurry | 20 | 5.0 | g | NA | NA | NA | Form A | NA | NA |
| 43 | THF/Water (1:2) | 2891-17-3 | Solid after | 35 | 5.0 | g | NA | 2.71 | NA | Form A | 90.4 | 96.57 |
| | | | drying at | | | | | | | | | |
| | | | 35° C. | | | | | | | | | |
| 44 | MeOH/Acetone/ | 2891-13-3 | Slurry | 20 | 165 | mg | 1 | NA | NA | Form I | NA | NA |
| | Water | | | | | | | | | | | |
| 45 | IPA/Water (1:1) | 2891-18-2 | Slurry | 20 | 102 | mg | 0 | NA | NA | Form A | NA | NA |
| | Crystallization | | | | | | | | | | | |
| 46 | IPA/Water (1:1) | 2891-18-3 | Solid | 20 | 102 | mg | 1 | NA | NA | Form A | NA | NA |
| | Crystallization | | | | | | | | | | | |
| 47 | IPA/Water (1:2) | 2891-20-1 | Solid after | 0 | 2.0 | g | 1 | 4.1 | 4.51 | Form A | 85.0 | |
| | Crystallization | | drying at | | | | | | | | | |
| | 0° C. | | 20° C. | | | | | | | | | |
| 48 | IPA/Water (1:2) | 2891-21-1 | Solid after | 10 | 2.0 | g | 1 | 4.04 | 4.95 | Form A | 77.0 | |
| | Crystallization | | diving at | | | | | | | | | |
| | 10° C. | | 20° C. | | | | | | | | | |
| 49 | IPA/Water (1:2) | 2891-19-1 | Solid after | 20 | 2.0 | g | 1 | NA | NA | Form A | 73.0 | 97.94 |
| | Crystallization | | drying at | | | | | | | | | |
| | 15° C. | | 20° C. | | | | | | | | | |
| 50 | IPA/Water (1:2) | 2891-22-1 | Solid after | 20 | 2.0 | g | 1 | 3.9 | 4.42 | Form A | 79.0 | |
| | Crystallization | | drying at | | | | | | | | | |
| | 20° C. | | 20° C. | | | | | | | | | |
| 51 | IPA/Water (1:2) | 2891-23-1 | Solid after | 30 | 2.0 | g | 1 | 4.1 | 4.37 | Form A + | 79.0 | |
| | Crystallization | | diving at | | | | | | | Unknown | | |
| | 30° C. | | 20° C. | | | | | | | | | |
| 52 | IPA/Water (1:2) | 2891-24-1 | Solid after | 40 | 2.0 | g | 1 | 0.99 | 1.23 | Form G | 87.0 | |
| | Crystallization | | drying at | | | | | | | | | |
| | 40° C. | | 20° C. | | | | | | | | | |
| 53 | IPA/Water (1:2) | 2891-25-1 | Solid after | 5 | 2.0 | g | 18 | 4.07 | 4.5 | Form A | 90.0 | 97.08 |
| | Conversion of | | drying at | | | | | | | | | |
| | Hemihydrate | | 20° C. | | | | | | | | | |
| 54 | IPA/Water (1:2) | 2891-26-1 | Solid after | −10 | 2.0 | g | 18 | 1.61 | 5.54 | Form H | 87.0 | 97.27 |
| | Crystallization −10° C. | | drying at | | | | | | | | | |
| | | | 20° C. | | | | | | | | | |
| 55 | IPA/Water (1:2) 30 | 2891-27-1 | Solid after | 5 | 2.0 | g | 18 | 4.05 | 4.65 | Form A | 88.0 | 97.03 |
| | mm addition of | | diving at | | | | | | | | | |
| | Water | | 20° C. | | | | | | | | | |
| 56 | IPA/Water (1:2) | 2891-28-1 | Solid after | 5 | 2.0 | g | 18 | 4.07 | 4.76 | Form A | 94.0 | 97.16 |
| | 120 min addition | | drying at | | | | | | | | | |
| | of Water | | 20° C. | | | | | | | | | |

NA—Not Available
ND—Not Determined
KF—water content determined by Karl Fischer water determination method

Table 9 below correlates OXY133 polymorph Forms A to I with impurities found in some of these crystal forms.

TABLE 9
HPLC Method: OXY133 Polymorph Impurities (CAD)

| | | | | | Crystal |
|---|---|---|---|---|---|
| | | | | | Form |
| Item | Method | Sample | Processing Point | Temp. (C.) | (XRPD) |
| 1 | OXY133 Anhydrous | 55352-23-07 | Solid | 20 | Form B |
| | From Medtronic | | | | |
| 2 | OXY133 | 82489-2-7-1 | Solid | 20 | Form A |
| | Monohydrate From | | | | |
| | Medtronic | | | | |
| 3 | Slurry of Form B in | 2891-1-1 | Slurry | 20 | Form E |
| | Water at 20° C. | | | | |
| 4 | Slurry of Form B in | 2891-1-4 | Slurry | 70 | Form E |
| | Water at 70° C. | | | | |
| 5 | Acetone/Water (1:1) | 2891-1-2 | Slurry | 20 | Form A |
| | at 20° C. | | | | |
| 6 | Acetone/Water (1:1) | 2891-1-5 | Slurry | 70 | Form C |
| | at 70° C. | | | | |
| 7 | MeOH/Water (1:1) | 2891-1-3 | Slurry | 20 | Form D |
| | at 20° C. | | | | |
| 8 | MeOH/Water (1:1) | 2891-2-1 | Slurry | 70 | Form D |
| | at 70° C. | | | | |
| 9 | Acetone/Water (1:1) | 2891-3-1 | Slurry | 20 | Form A + |
| | Overhead stirring | | | | Form B |
| 10 | Acetone/Water (1:1) | 2891-3-2 | Slurry | 20 | Form A |
| | Overhead stirring | | | | |
| 11 | Acetone/Water (1:1) | 2891-3-4 | Solid after | 50 | Form A |
| | Overhead stirring | | drying at | | |
| | | | 50° C. | | |
| 12 | Acetone/Water (1:1) | 2891-4-1 | Slurry | 30 | Form C |
| | 30° C. | | | | |
| 13 | Acetone/Water (1:1) | 2891-4-2 | Slurry | 30 | Form C |
| | 30° C. | | | | |
| 14 | Acetone/Water (1:1) | 2891-4-3 | Slurry | 30 | Form C |
| | 30° C. | | | | |
| 15 | Acetone/Water (1:1) | 2891-4-4 | Solid after | 30 | Form C |
| | 30° C. | | drying at | | |
| | | | 35° C. | | |
| 16 | Acetone/Water (1:1) | 2891-5-1 | Slurry | 40 | Form C |
| | 40° C. | | | | |
| 17 | Acetone/Water (1:1) | 2891-5-2 | Slurry | 40 | Form C |
| | 40° C. | | | | |
| 18 | Acetone/Water 1:1) | 2891-5-3 | Solid after | 40 | Form C |
| | 40° C. | | drying at | | |
| | | | 35° C. | | |
| 19 | Acetone/Water (1:1) | 2891-6-1 | Slurry | 50 | Form C |
| | 50° C. | | | | |
| 20 | Acetone/Water (1:1) | 2891-6-2 | Slurry | 50 | Form C |
| | 50° C. | | | | |
| 21 | Acetone/Water (1:1) | 2891-6-3 | Solid after | 50 | Form C |
| | 50° C. | | drying at | | |
| | | | 35° C. | | |
| 22 | Acetone/Water (1:1) | 2891-7-1 | Slurry | 60 | Form C |
| | 60° C. | | | | |
| 23 | Acetone/Water (1:1) | 2891-7-2 | Slurry | 60 | Form C |
| | 60° C. | | | | |
| 24 | Acetone/Water (1:1) | 2891-7-3 | Slurry | 10 | Form C |
| | 60° C. | | | | |
| 25 | Acetone/Water (1:1) | 2891-7-4 | Slurry | 20 | Form C |
| | 60° C. | | | | |
| 26 | Acetone/Water (1:1) | 2891-7-5 | Solid after | 20 | Form C |
| | 60° C. | | drying at | | |
| | | | 35° C. | | |
| 27 | Acetone/Water (1:1) | 2891-8-1 | Slurry | 0 | Form A + |
| | 0° C. | | | | Form B |
| 28 | Acetone/Water (1:1) | 2891-8-2 | Slurry | 0 | Form A |
| | 0° C. | | | | |
| 29 | Acetone/Water (1:1) | 2891-8-3 | Solid after | 35 | Form A |
| | 0° C. | | drying at | | |
| | | | 35° C. | | |
| 30 | Acetone/Water (1:1) | 2891-9-1 | Slurry | 10 | Form A + |
| | 10° C. | | | | Form B |
| 31 | Acetone/Water (1:1) | 2891-9-2 | Slurry | 10 | Form A |
| | 10° C. | | | | |
| 32 | Acetone/Water (1:1) | 2891-9-3 | Solid after | 35 | Form A |
| | 10° C. | | drying at | | |
| | | | 35° C. | | |
| 33 | Acetone/Water (1:1) | 2891-10-1 | Slurry | 25 | Form A + |
| | 25° C. | | | | Form B |
| 34 | Acetone/Water (1:1) | 2891-10-2 | Slurry | 25 | Form A |
| | 25° C. | | | | |
| 35 | Acetone/Water (1:1) | 2891-10-3 | Solid after | 35 | Form A |
| | 25° C. | | drying at | | |
| | | | 35° C. | | |
| 36 | Acetone/Water | 2891-12-1 | Slurry after | 15 | Form F |
| | Dissolution/Precipitation | | charging water | | |
| 37 | Acetone/Water | 2891-12-2 | Slurry | 5 | Form F |
| | Dissolution/Precipitation | | | | |
| 38 | Acetone/Water | 2891-12-3 | Slurry | 5 | Form F |
| | Dissolution/Precipitation | | | | |
| 39 | Acetone | 2891-14-1 | Solid | 20 | Form I |
| | Recrystallization | | | | |
| 40 | THF/Acetone/Water | 2891-16-1 | Solid before | 35 | Form A |
| | Crystallization | | drying | | |
| 41 | THF/Acetone/Water | 2891-16-2 | Solid after | 35 | Form A |
| | Crystallization | | drying at | | |
| | | | 35° C. | | |
| 42 | THF/Water (1:2) | 2891-17-1 | Oil/Slurry | 20 | Form A |
| 43 | THF/Water (1:2) | 2891-17-3 | Solid after | 35 | Form A |
| | | | drying at | | |
| | | | 35° C. | | |
| 44 | MeOH/Acetone/Water | 2891-13-3 | Slurry | 20 | Form I |
| 45 | IPA/Water (1:1) | 2891-18-2 | Slurry | 20 | Form A |
| | Crystallization | | | | |
| 46 | IPA/Water (1:1) | 2891-18-3 | Solid | 20 | Form A |
| | Crystallization | | | | |
| 47 | IPA/Water (1:2) | 2891-20-1 | Solid after | 0 | Form A |
| | Crystallization 0° C. | | drying at | | |
| | | | 20° C. | | |
| 48 | IPA/Water (1:2) | 2891-21-1 | Solid after | 10 | Form A |
| | Crystallization 10° C. | | drying at | | |
| | | | 20° C. | | |
| 49 | IPA/Water (1:2) | 2891-19-1 | Solid after | 20 | Form A |
| | Crystallization 15° C. | | drying at | | |
| | | | 20° C. | | |
| 50 | IPA/Water (1:2) | 2891-22-1 | Solid after | 20 | Form A |
| | Crystallization 20° C. | | drying at | | |
| | | | 20° C. | | |
| 51 | IPA/Water (1:2) | 2891-23-1 | Solid after | 30 | Form A + |
| | Crystallization 30° C | | drying at | | Unknown |
| | | | 20° C. | | |
| 52 | IPA/Water (1:2) | 2891-24-1 | Solid after | 40 | Form G |
| | Crystallization 40° C | | drying at | | |
| | | | 20° C. | | |
| 53 | IPA/Water (1:2) | 2891-25-1 | Solid after | 5 | Form A |
| | Conversion of | | drying at | | |
| | Hemihydrate | | 20° C. | | |
| 54 | IPA/Water (1:2) | 2891-26-1 | Solid after | −10 | Form H |
| | Crystallization −10° C. | | diving at | | |
| | | | 20° C. | | |
| 55 | IPA/Water (1:2) 30 | 2891-27-1 | Solid after | 5 | Form A |
| | min addition of | | drying at | | |
| | Water | | 20° C. | | |
| 56 | IPA/Water (1:2) | 2891-28-1 | Solid after | 5 | Form A |
| | 120 min addition | | drying at | | |
| | of Water | | 20° C. | | |

Area Percent (AP)

| | OXY | | | |
|---|---|---|---|---|
| | 133 | Imp-1 | Imp-2 | Imp-3 |
| rt | 12.97 | 14.64 | 17.12 | 17.3 |
| (min) | | | | |
| rtt | 1.00 | 1.13 | 1.32 | 1.33 |
| | 96.88 | 2.35 | 0.77 | ND |
| | 96.79 | 2.44 | 0.77 | ND |
| | 96.75 | 2.30 | 0.92 | ND |
| | 96.97 | 2.25 | 0.78 | ND |
| | 98.87 | ND | 1.13 | ND |
| | 99.31 | ND | 0.67 | ND |
| | 96.57 | 2.18 | 0.76 | 0.48 |
| | 97.94 | 1.15 | 0.91 | ND |
| | 97.08 | 2.12 | 0.80 | ND |
| | 97.27 | 2.02 | 0.72 | ND |
| | 97.03 | 2.33 | 0.65 | ND |
| | 97.16 | 2.12 | 0.72 | ND |

NA—Not Available
ND—Not Determined
rt—Retention Time
rrt—Relative Retention Time

The HPLC-CAD data summarized in Tables 8 and 9 above was collected on a HPLC Agilent 1100 instrument equipped with a Waters XBridge phenyl, 4.6 mm by 150 mm, 3.5 μm column, at a column temperature of 40±2° C., the mobile phases, MPA and MPB were 100% water and methanol (MeOH), respectively, and the flow rate was 1.0 mL/min.

The CAD equipment utilized for the experimental work of this disclosure was Dionex Corona ultra RS, wherein the unit settings included a range of 100 pA, offset of 0, and no filter. The CAD's nebulizer temperature was 35±5° C. and the gas pressure about 35 psi. The HPLC's sample tray was kept at ambient temperature, the injection volume was 5 μl, the needle wash used was the method diluent, the run time 35 minutes and the retention time for OXY133 approximately 13.1 minutes.

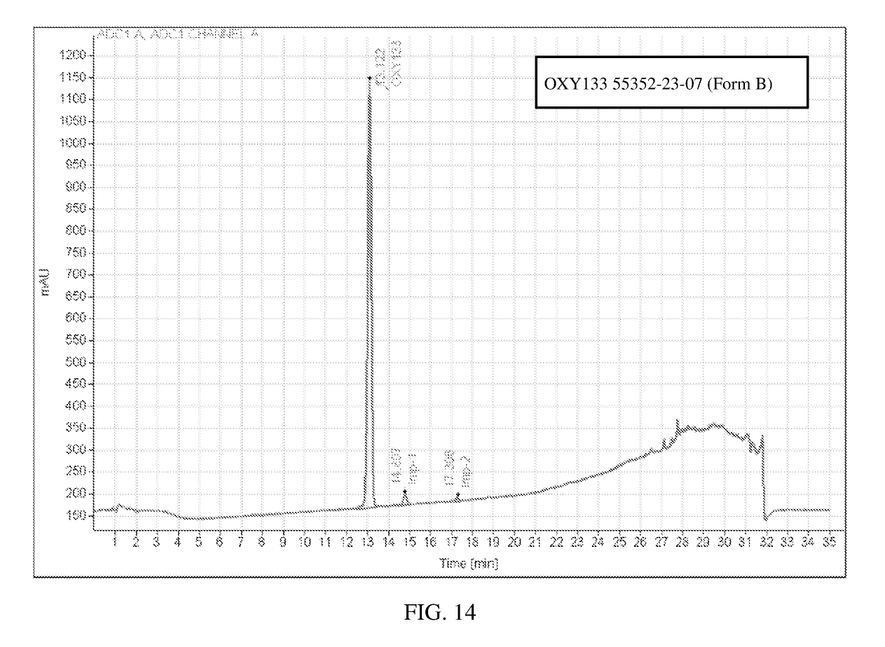

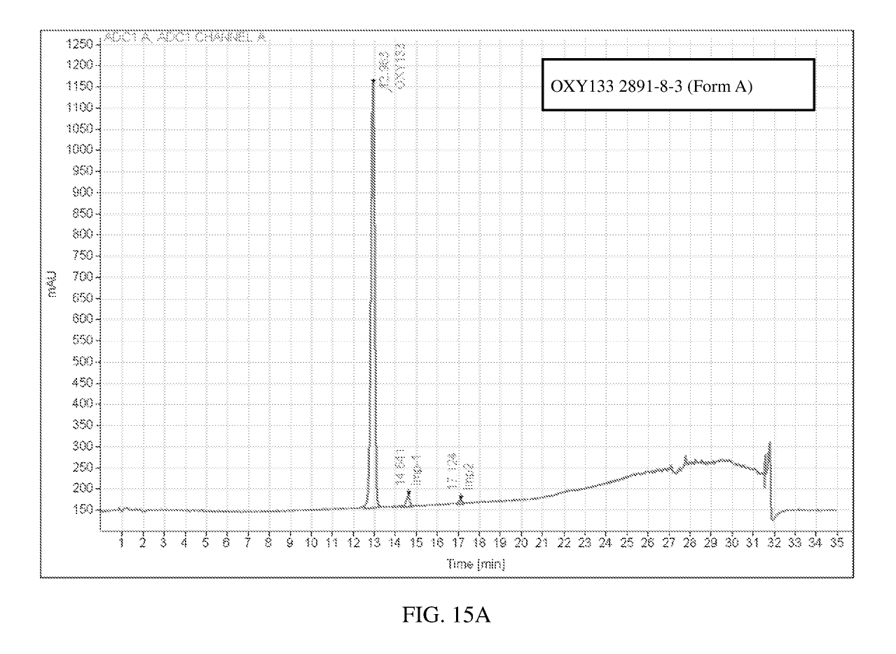

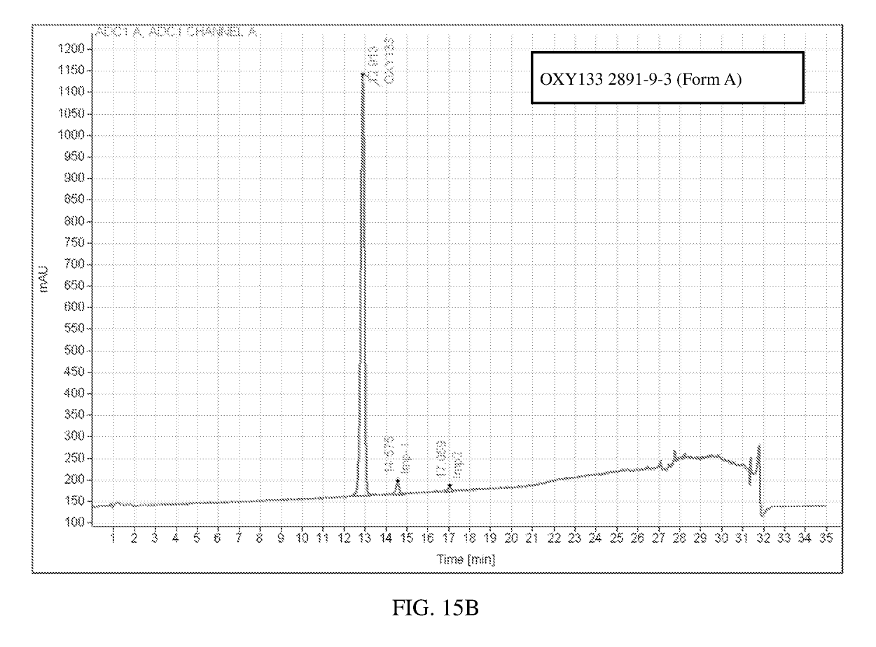

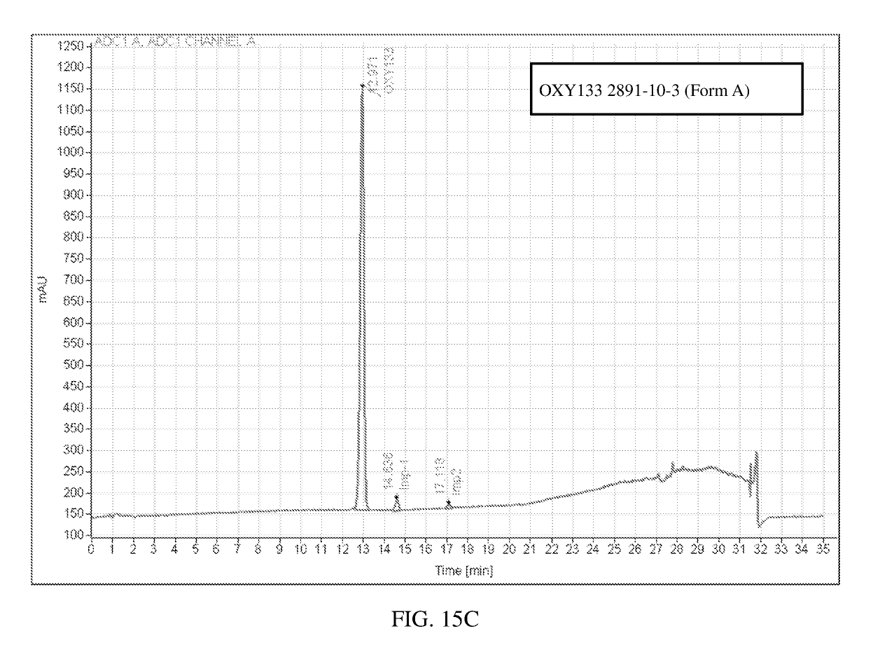

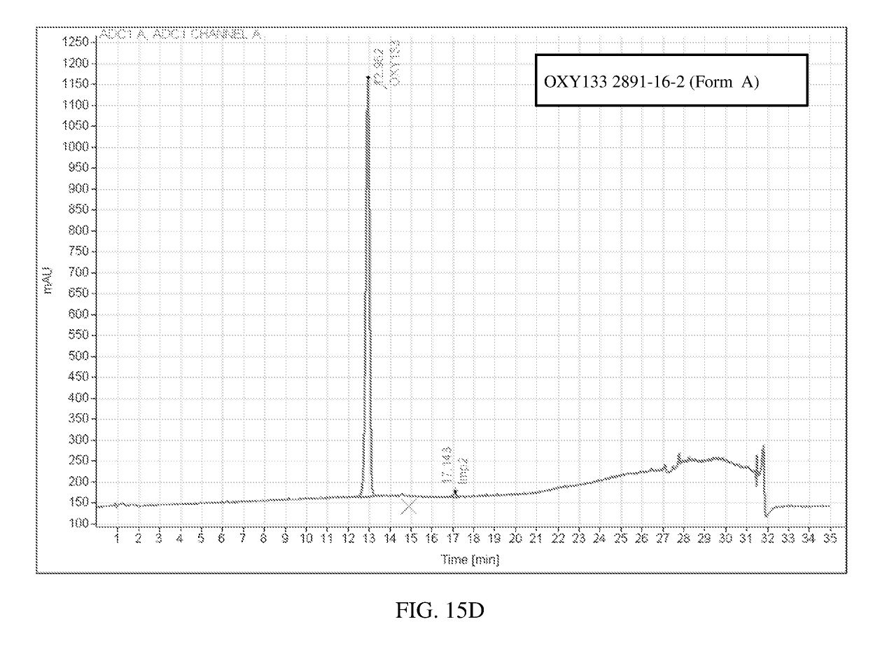

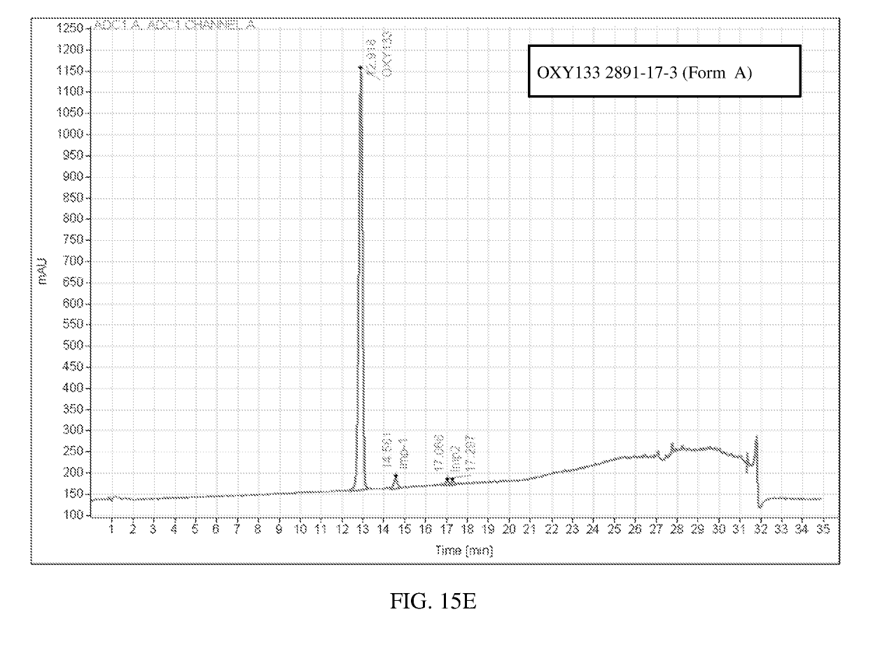

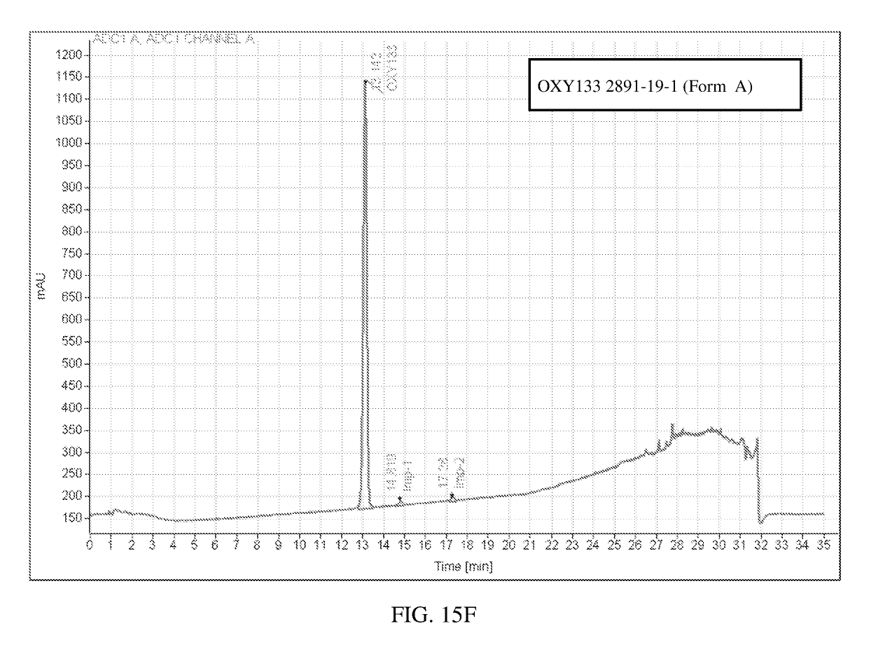

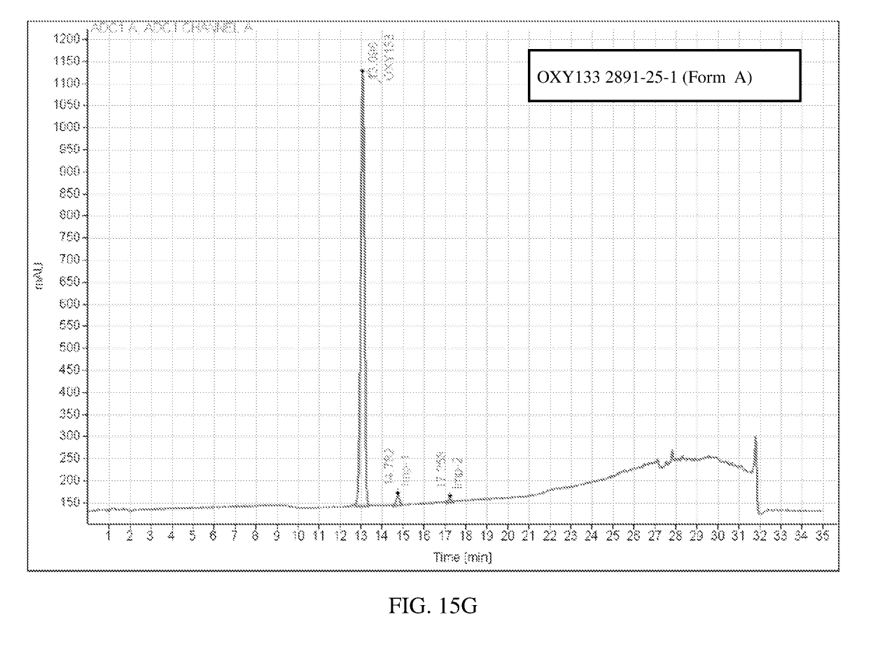

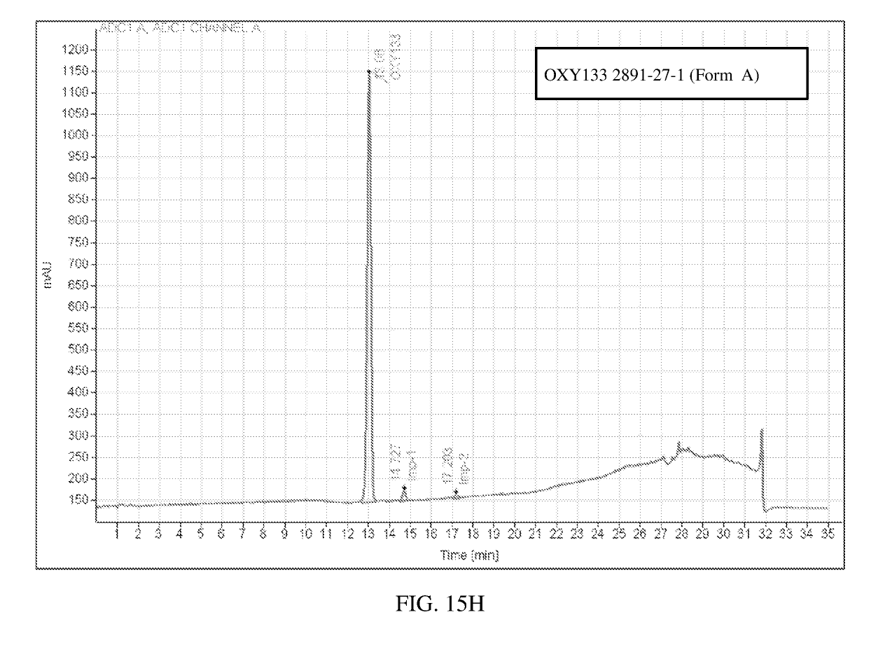

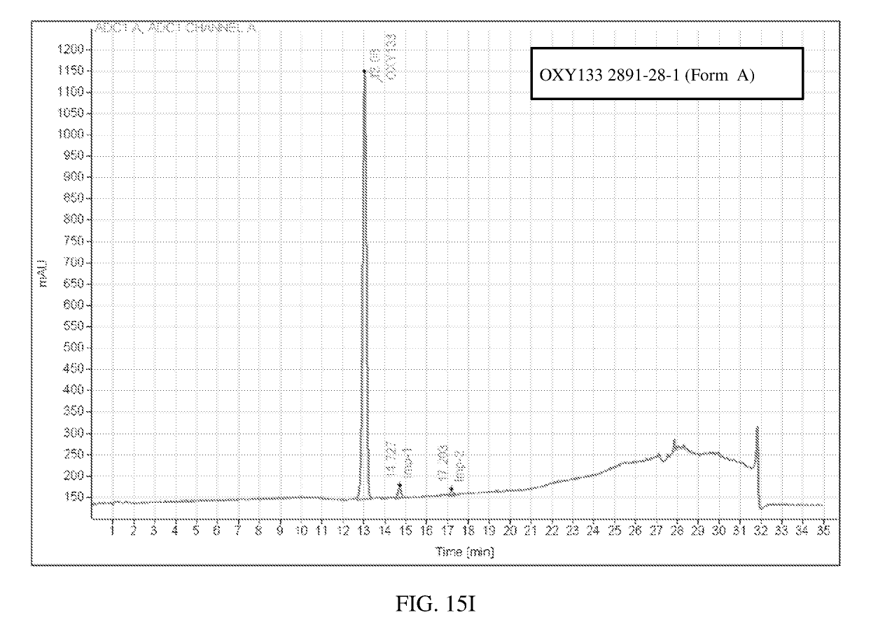

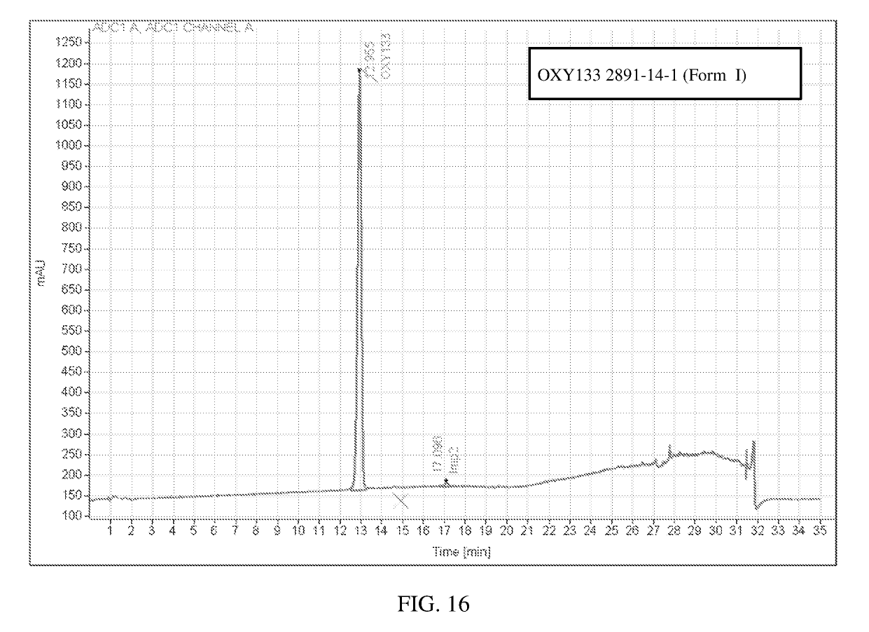

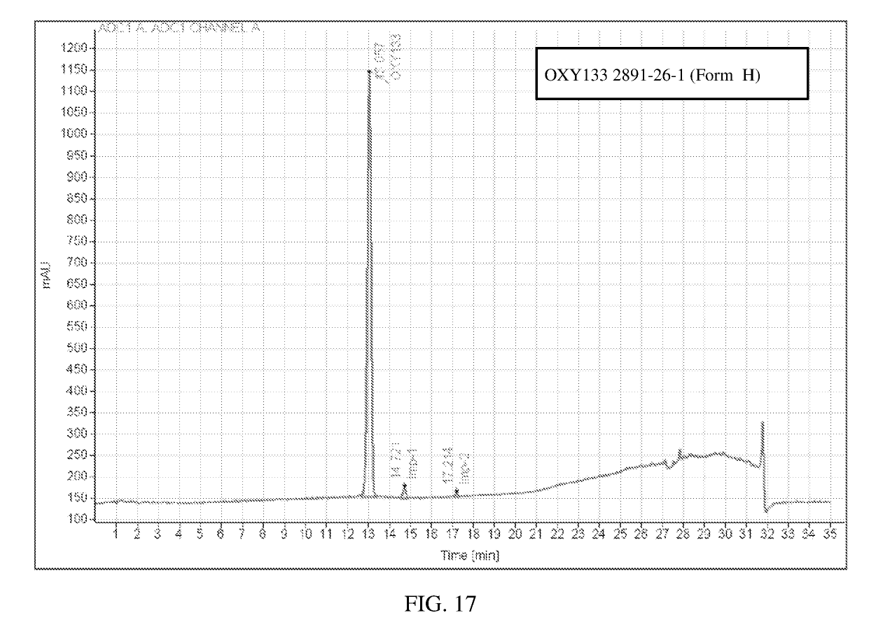

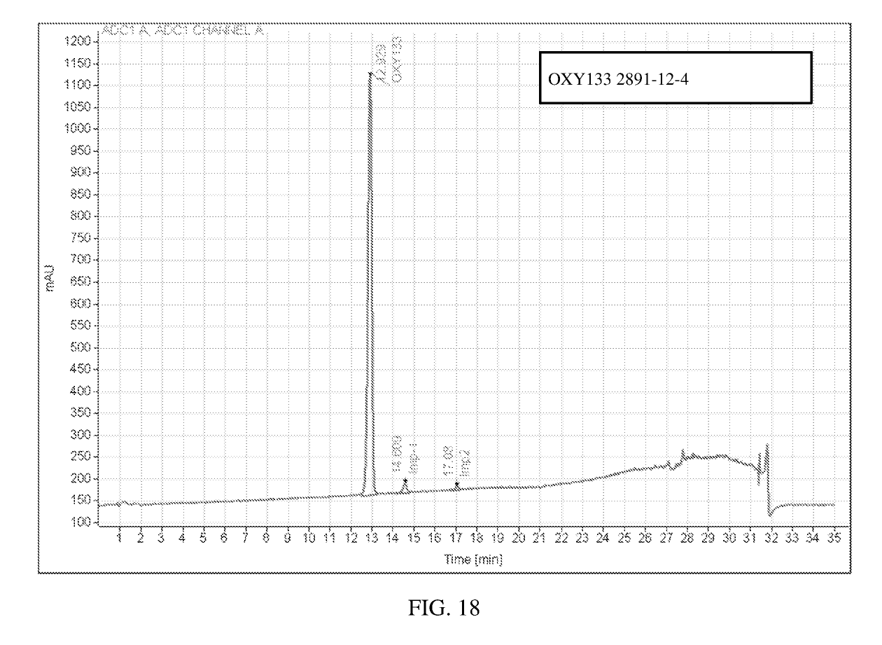

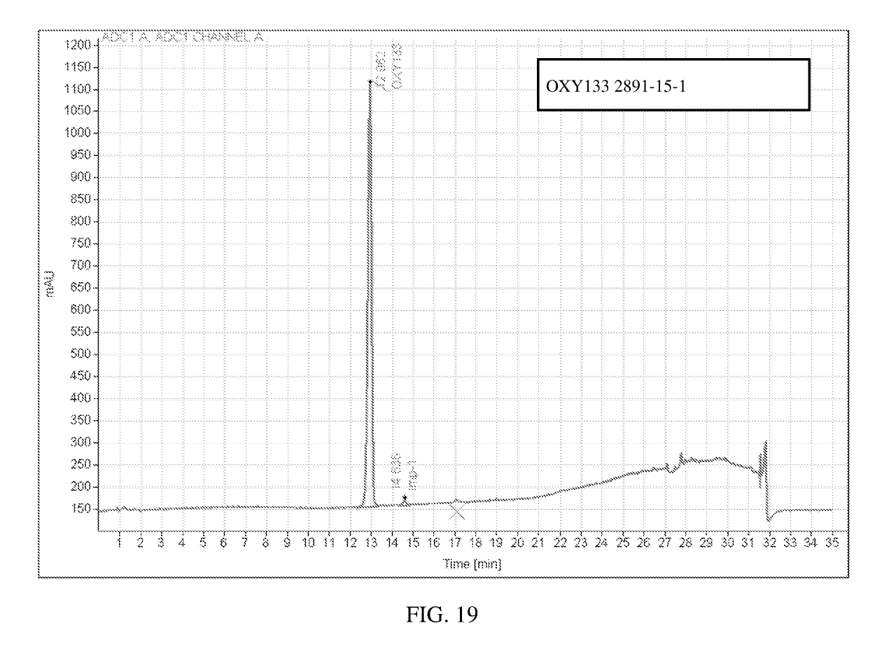

In various embodiments, some of the HPLC-CAD single injection results are illustrated in FIGS. 14-19. The data collected from these single injection results and associated with each signal is summarized in Table 10. HPLC-CAD data identifying OXY133 polymorph B is found at FIG. 14. HPLC-CAD data identifying OXY133 polymorph A is illustrated in FIGS. 15A, 15B, 15C, 15D, 15E, 15F, 15G, 15H and 15I. HPLC-CAD data identifying OXY133 polymorphs H and I is depicted in FIGS. 16 and 17, respectively. FIGS. 18 and 19 illustrate HPLC-CAD data for OXY133 samples 2891-12-4 and 1891-15-1.

TABLE 10
Signal: ADC1 A, ADC1 Channel A

| FIG./Poymorph | | Peak | | Area | Resolution | | Compound |
|---|---|---|---|---|---|---|---|
| Form | RT (min) | Height | Area | Percent | S/N | Tailing | Name |
| FIG. 14 | 13.12 | 977.8 | 12035.04 | 96.88 | NA | 0.9 | OXY133 |
| Form B | 14.81 | 26.1 | 292.059 | 2.351 | 5.9 | 1.2 | Imp-1 |
| | 17.31 | 10.1 | 95.554 | 0.769 | 14.1 | 0.8 | Imp-2 |
| FIG. 15A | 12.96 | 1004.9 | 12862.927 | 96.79 | NA | 0.9 | OXY133 |
| Form A | 14.64 | 27.4 | 323.718 | 2.436 | 5.9 | 0.8 | Imp-1 |
| | 17.12 | 9.9 | 102.861 | 0.774 | 12.0 | 0.9 | Imp-2 |
| FIG. 15B | 12.91 | 971.9 | 12469.852 | 96.747 | NA | 0.9 | OXY133 |
| Form A | 14.58 | 26.4 | 300.637 | 2.332 | 5.9 | 1.1 | Imp-1 |
| | 17.06 | 9.9 | 118.685 | 0.921 | 11.1 | 0.9 | Imp-2 |
| FIG. 15C | 12.97 | 992.3 | 12903.733 | 96.972 | NA | 0.9 | OXY133 |
| Form A | 14.64 | 25.5 | 299.795 | 2.253 | 5.8 | 1.0 | Imp-1 |
| | 17.12 | 8.6 | 103.15 | 0.775 | 14.6 | 0.9 | Imp-2 |
| FIG. 18 | 12.93 | 958.6 | 12543.434 | 96.705 | NA | 0.9 | OXY133 |
| Form 2891-12-4 | 14.61 | 23.6 | 308.917 | 2.382 | 5.8 | 0.8 | Imp-1 |
| | 17.08 | 10.1 | 118.415 | 0.913 | 13.2 | 0.9 | Imp-2 |
| FIG. 16 | 12.95 | 1012.6 | 13252.109 | 98.872 | NA | 0.9 | OXY133 |
| Form I | 17.10 | 10.5 | 151.170 | 1.128 | 14.8 | 0.8 | Imp-2 |
| FIG. 19 | 12.96 | 961.1 | 12444.046 | 98.778 | NA | 0.9 | OXY133 |
| Form 2891-15-1 | 14.64 | 12.4 | 153.953 | 1.222 | 6.5 | 0.9 | Imp-1 |
| FIG. 15D | 12.96 | 995.2 | 12999.925 | 99.314 | NA | 0.9 | OXY133 |
| Form A | 17.14 | 9.3 | 89.849 | 0.686 | 17.9 | 0.9 | Imp-2 |
| FIG. 15E | 12.92 | 991.0 | 12673.089 | 96.569 | NA | 0.9 | OXY133 |
| Form A | 14.58 | 24.0 | 286.633 | 2.184 | 5.8 | 0.9 | Imp-1 |
| | 17.07 | 9.0 | 100.206 | 0.764 | 11.4 | 0.9 | Imp-2 |
| | 17.3 | 8.0 | 63.488 | 0.484 | 1.5 | 1.6 | NA |
| FIG. 15F | 13.14 | 962.4 | 11702.109 | 97.937 | NA | 0.9 | OXY133 |
| Form A | 14.82 | 11.2 | 137.895 | 1.154 | 5.8 | 1.0 | Imp-1 |
| | 17.28 | 9.7 | 108.565 | 0.909 | 11.3 | 1.3 | Imp-2 |
| FIG. 15G | 13.10 | 980.6 | 12547.018 | 97.083 | NA | 0.9 | OXY133 |
| Form A | 14.78 | 23.2 | 274.022 | 2.120 | 6.1 | 0.9 | Imp-1 |
| | 17.26 | 8.9 | 102.958 | 0.797 | 9.4 | 1.0 | Imp-2 |
| FIG. 17 | 13.06 | 989.6 | 12586.536 | 97.265 | NA | 0.9 | OXY133 |
| Form H | 14.72 | 23.2 | 261.195 | 2.018 | 6.1 | 1.0 | Imp-1 |
| | 17.21 | 8.5 | 92.714 | 0.716 | 10.6 | 1.0 | Imp-2 |
| FIG. 15H | 13.07 | 992.6 | 12749.081 | 97.029 | NA | 0.9 | OXY133 |
| Form A | 14.74 | 26.7 | 305.567 | 2.326 | 5.9 | 0.8 | Imp-1 |
| | 17.25 | 10.0 | 84.825 | 0.646 | 16.6 | 1.4 | Imp-2 |
| FIG. 15I | 13.06 | 999.4 | 12778.192 | 97.158 | NA | 0.9 | OXY133 |
| Form A | 14.73 | 24.5 | 278.862 | 2.120 | 5.6 | 1.1 | Imp-1 |
| | 17.20 | 9.4 | 94.863 | 0.721 | 10.3 | 1.2 | Imp-2 |

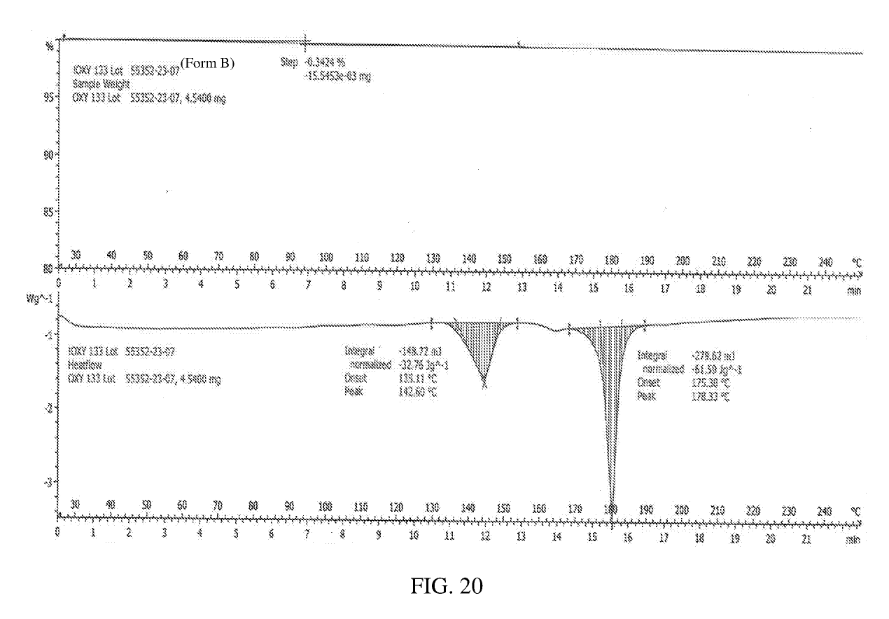

Differential scanning calorimetry (DSC) and thermo-gravimetric analysis (TGA) were also collected for some of the OXY133 polymorphs. The equipment utilized to collect the DSC/TGA data was a Mettler Toledo DSC 2 equipped with an aluminum 40 μL crimped pan with a pin hole and the ramp rate was 10° C./min. DSC/TGA thermograms identifying several OXY133 polymorphs are illustrated in FIGS. 20-25.

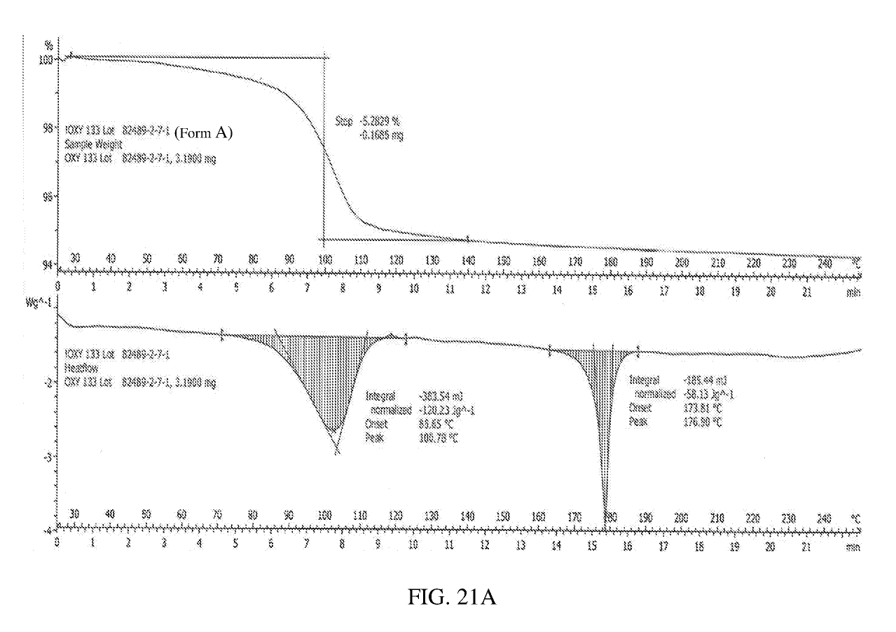

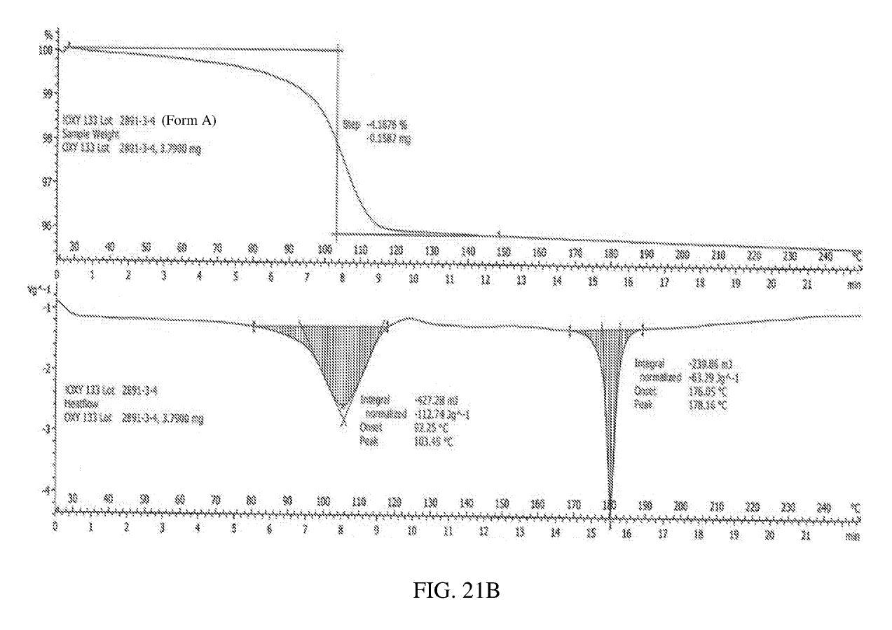

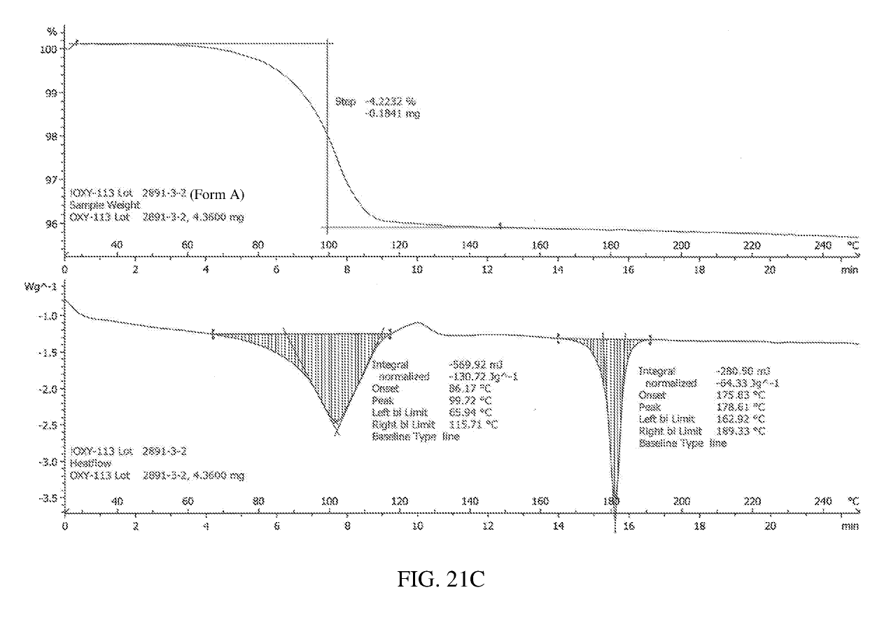

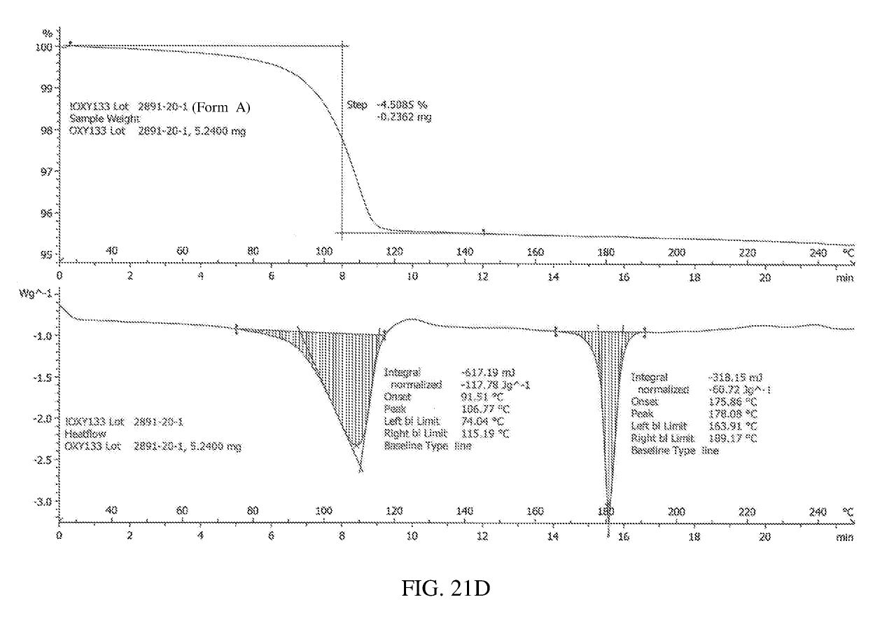

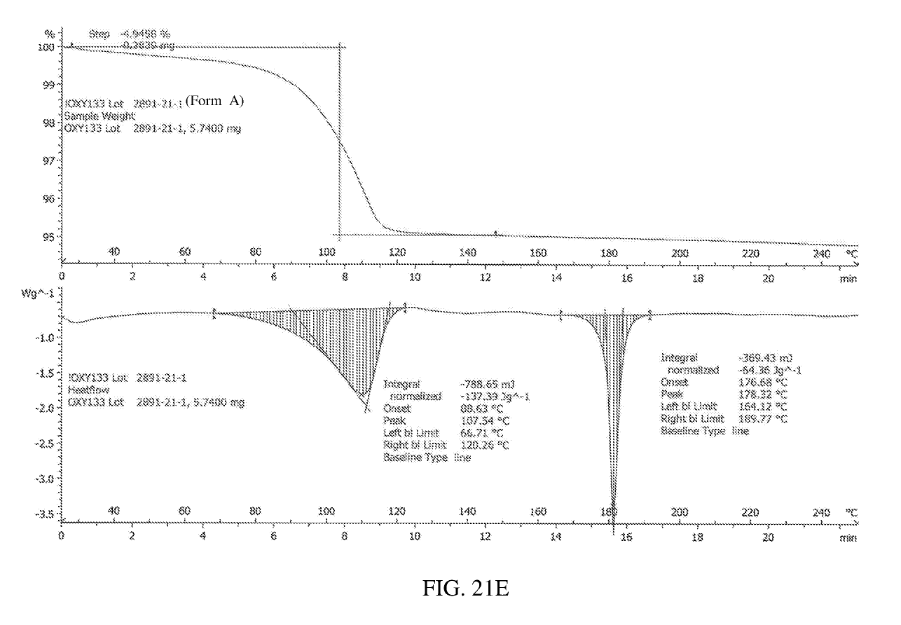

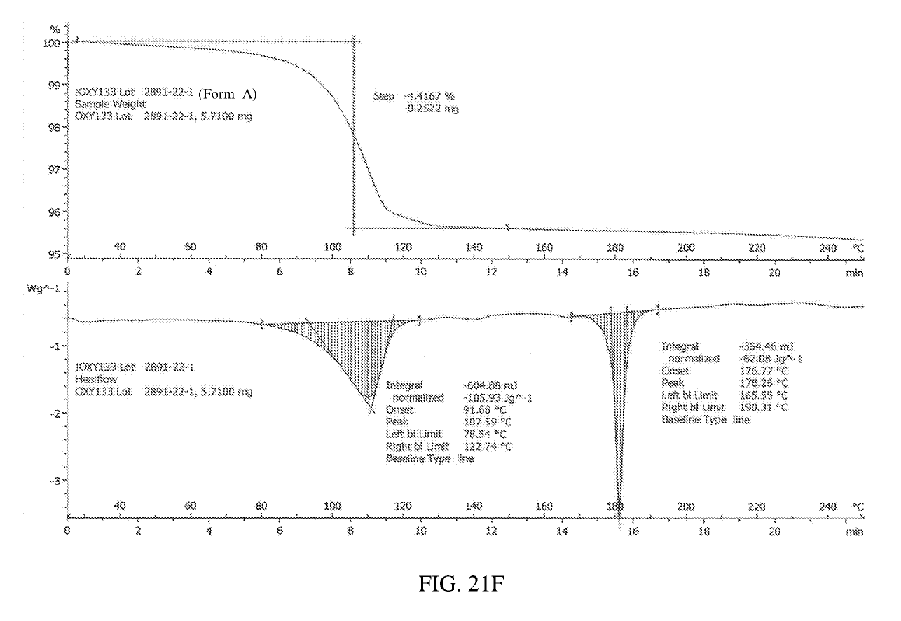

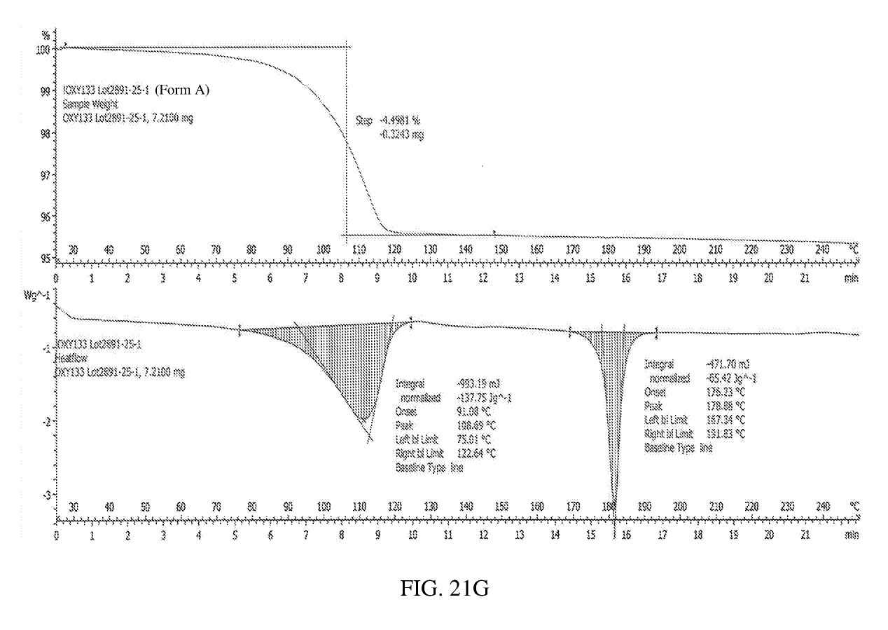

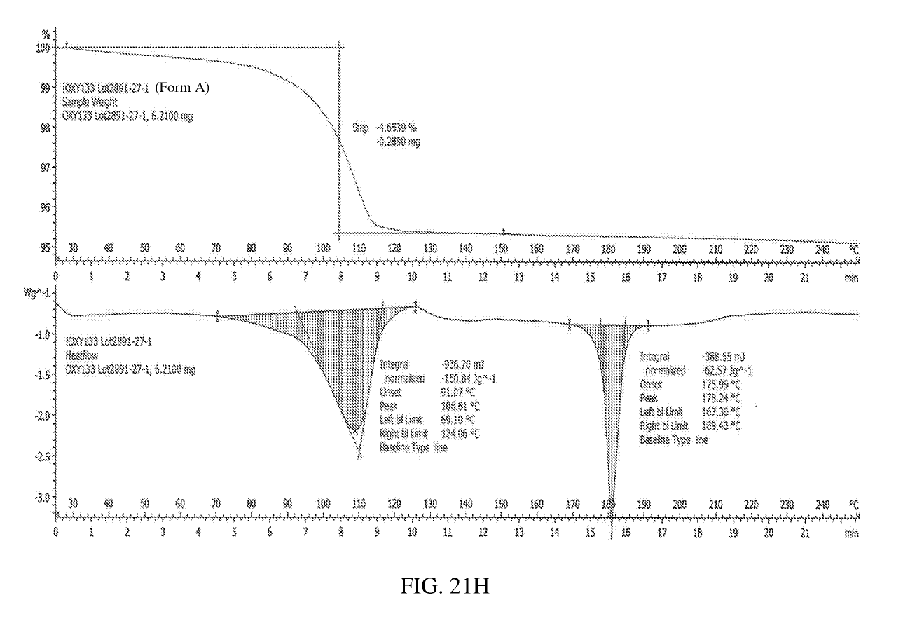

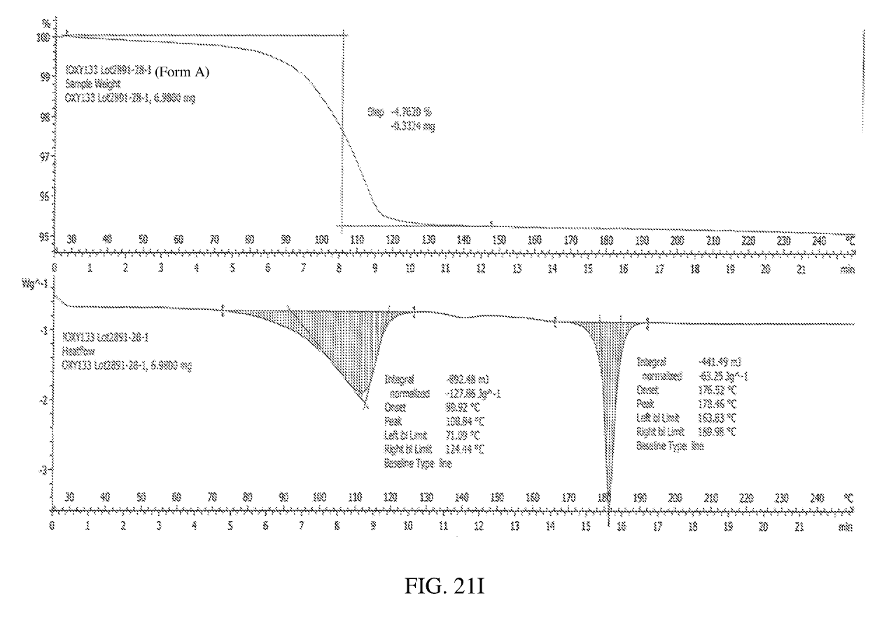

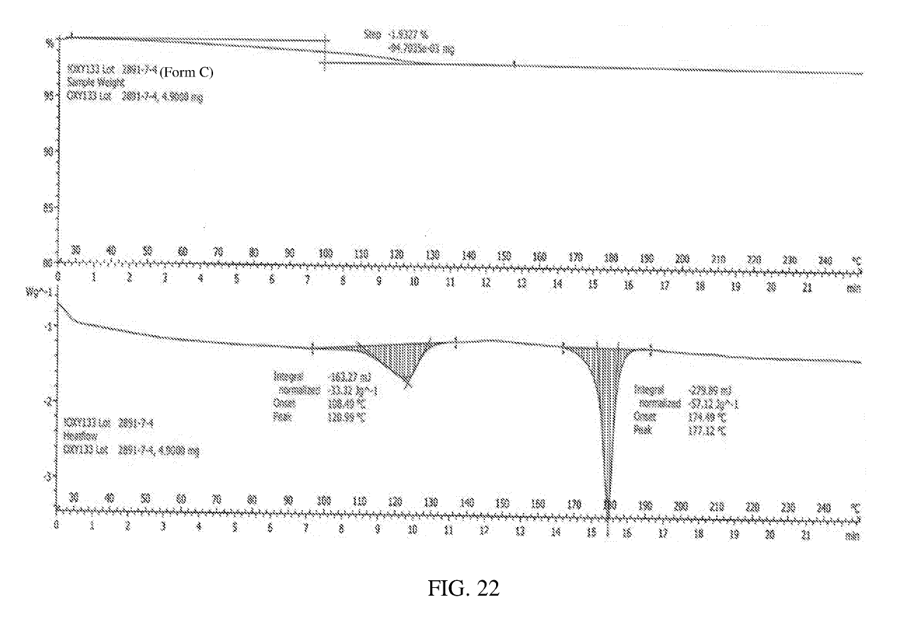

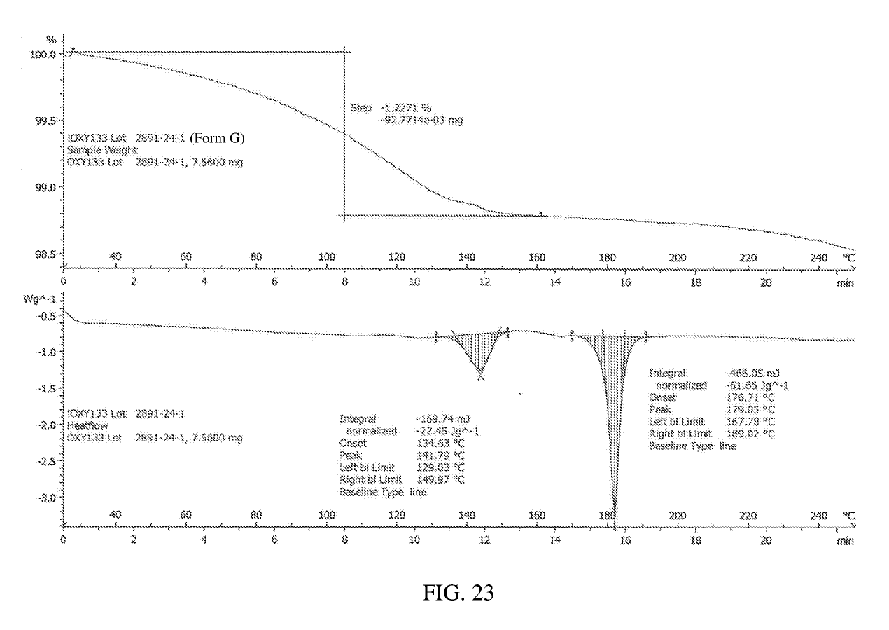

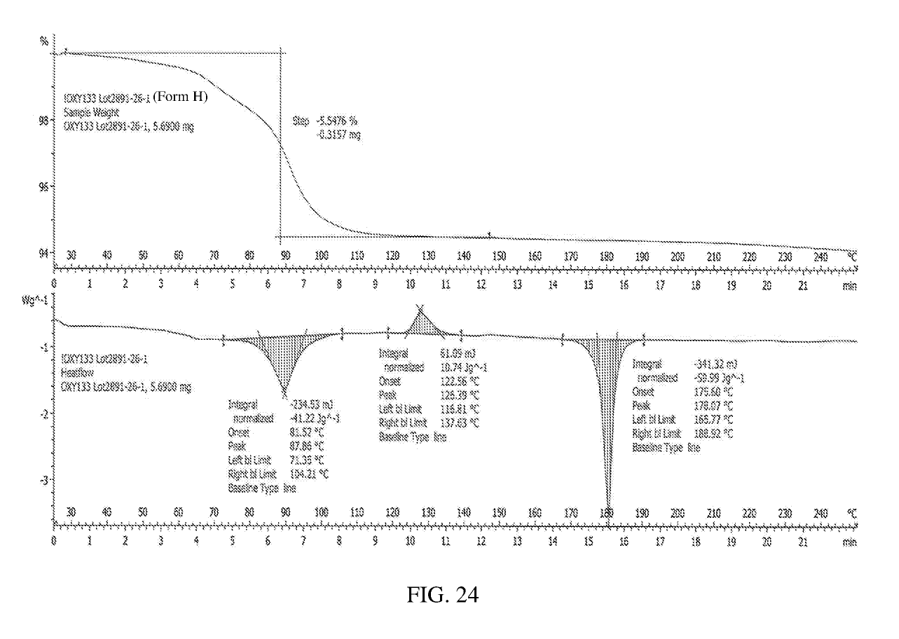

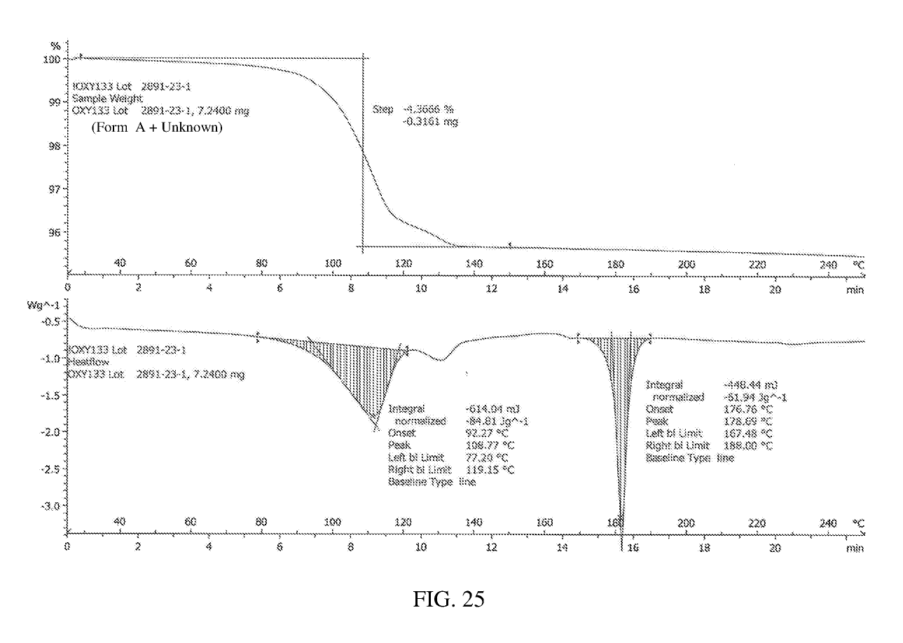

More particularly, FIG. 20 is a DSC-TGA thermogram of OXY133 polymorph Form B; FIGS. 21A, 21B, 21C, 21D, 21E, 21F, 21G, 21H, and 21I are DSC-TGA thermograms of OXY133 polymorph Form A; FIG. 22 is a DSC-TGA thermogram of OXY133 polymorph Form C; FIG. 23 is a DSC-TGA thermogram of OXY133 polymorph Form G; FIG. 24 is a DSC-TGA thermogram of OXY133 polymorph Form H; and FIG. 25 is a DSC-TGA thermogram of OXY133 polymorph Form A and unknown.

In various other embodiments, a method is provided for preparing an OXY133 polymorph, the method including subjecting a slurry of anhydrous OXY133 to conditions sufficient to convert anhydrous OXY133 to the OXY133 polymorph selected from polymorph Form A, polymorph Form B, polymorph Form C, polymorph Form D, polymorph Form E, polymorph Form F, polymorph Form G, polymorph Form H, polymorph Form I or a mixture thereof, wherein OXY133 is prepared by reacting a diol having the formula:

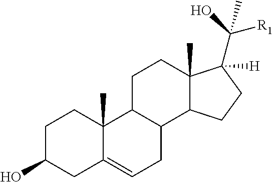

with borane, hydrogen peroxide and tetrahydrofuran to form the oxysterol or a pharmaceutically acceptable salt, hydrate or solvate thereof having the formula:

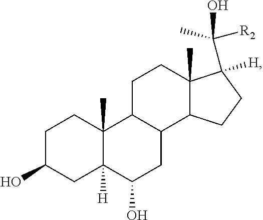

wherein R1 and R2 comprise a hexyl group and the diol comprises (3S,8S,9S,10R,13R,14S,17R)-10,13-dimethyl-17-[(S)-2-hydroxyoctan-yl]-2,3,4,7,8,9,11,12,14,15,16,17-dodecahydro-1H-cyclopenta[a]phenanthren-3-ol (OXY133).

These and other aspects of the present application will be further appreciated upon consideration of the following Examples, which are intended to illustrate certain particular embodiments of the application but are not intended to limit its scope, as defined by the claims.

### EXAMPLES

### Example 1

Preparation from Pregnenolone Acetate

8.25 mL n-hexylmagnesium chloride (2 M, 16.5 mmol) in was added to a solution of pregnenolone acetate in tetrahydrofuran under vigorous electromagnetic stirring and ice bath cooling. The pregnenolone acetate solution contained 1.79 g compound 1, pregnenolone acetate, (5 mmol) in 4.5 mL tetrahydrofuran. The addition took place over 2 minutes. After addition was completed, the mixture was stirred at room temperature for 3.5 hours, at which point the mixture had turned to a gel. The gel was then digested with a mixture of saturated aqueous NH₄Cl and MTBE (methyl tertiary-butyl ether). The organic layer was separated, washed with water three times and evaporated. The residue was separated by silica gel column chromatography using an EtOAc (ethyl acetate)/petroleum ether mixture (ratio 70/30) to give compound 2, a diol, as a white solid. 1.29 g (3.21 mmol) of the solid diol was extracted for a 64% isolated yield. The reaction is shown below in A:

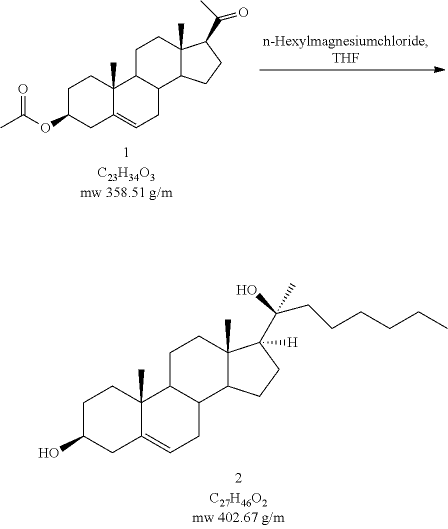 [SMILES: CC(=O)O[C@H]1CC[C@@]2(C)C(=CCC3C2CC[C@@]2(C)C3CC[C@@H]2C(C)=O)C1 ; CCCCCC[C@](C)(O)[C@H]1CCC2C3CC=C4C[C@@H](O)CC[C@]4(C)C3CC[C@@]21C]

The ¹H NMR data of the diol in CDCl₃ at 400 MHz illustrated the following: δ: 0.8-1.9 (40H), 1.98 (m, 1H), 2.09 (m, 1H), 2.23 (m, 1H), 2.29 (m, 1H), 3.52 (m, 1H), 5.35 (m, 1H) in FIG. 6. The ¹³C NMR data of the diol in CDCl₃ at 100 MHz illustrated the following: d: 13.6, 14.1, 19.4, 20.9, 22.4, 22.6, 23.8, 24.2, 26.4, 30.0, 31.3, 31.6, 31.8, 31.9, 36.5, 37.3, 40.1, 42.3, 42.6, 44.0, 50.1, 56.9, 57.6, 71.7, 75.2, 121.6, 140.8.

The diol created has an IUPAC name of (3S,8S,9S,10R,13R,14S,17R)-10,13-dimethyl-17-[(S)-2-hydroxyoctan-yl]-2,3,4,7,8,9,11,12,14,15,16,17-dodecahydro-1H-cyclopenta[a]phenanthren-3-ol.

### Example 2

Preparation from Pregnenolone

Alternatively to Example 1, compound 2 of reaction scheme A above can be prepared from pregnenolone shown below in B utilizing the same procedure as utilized for the conversion of compound 1 to compound 2. In this procedure 10 g of pregnenolone was converted to 7.05 g of compound 2, which accounted for a 55% yield.

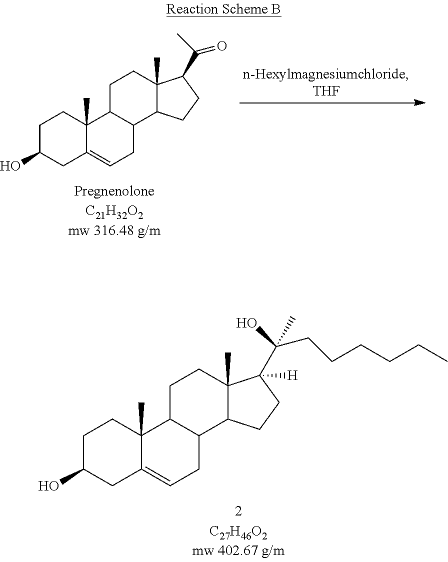 [SMILES: CC(=O)[C@H]1CCC2C3CC=C4C[C@@H](O)CC[C@]4(C)C3CC[C@@]21C ; CCCCCC[C@](C)(O)[C@H]1CCC2C3CC=C4C[C@@H](O)CC[C@]4(C)C3CC[C@@]21C]

2500 mL of n-hexylmagnesium chloride (2 M, 5 mol) was charged to a reactor and the solution was cooled to −5° C. A solution of pregnenolone acetate in tetrahydrofuran was charged to the reactor at a rate which maintained the internal reaction temperature below 1° C. The pregnenolone solution contained 500 g pregnenolone (1.4 mol) in 8 liters tetrahydrofuran. After the addition was complete, the mixture was held at 0° C. for 1 hour then allowed to warm to room temperature overnight. The reaction mixture had become a solid, gelatinous mass. 2 liters of additional tetrahydrofuran was added followed by 10 ml of glacial acetic acid. The reaction mixture was cooled to 5° C. and quenched by the addition of 350 ml of glacial acetic acid which gave a solution. The reaction mixture was concentrated under reduced pressure to a thick syrup. The compound was dissolved in dichloromethane, washed with water and finally washed with saturated sodium bicarbonate. The organic layer was concentrated under reduced pressure to an amber oil. Mass recovery was about 800 grams. The crude material was utilized as is in the next step.

The diol created has an IUPAC name of (3S,8S,9S,10R,13R,14S,17R)-10,13-dimethyl-17-[(S)-2-hydroxyoctan-yl]-2,3,4,7,8,9,11,12,14,15,16,17-dodecahydro-1H-cyclopenta[a]phenanthren-3-ol.

### Example 3

The crude hexyl diol product (800 grams) was dissolved in 8 liters of tetrahydrofuran, charged to a reactor, and was cooled to −5° C. 6300 mL of borane-tetrahydrofuran complex (1 M, 6.3 moles, 4.5 equivalents) in tetrahydrofuran was charged at a rate which maintained the internal reaction temperature below 1° C. Once the addition was complete, the reaction mixture was stirred at 0° C. for 1.5 hours then allowed to warm to room temperature overnight. The reaction is shown below.

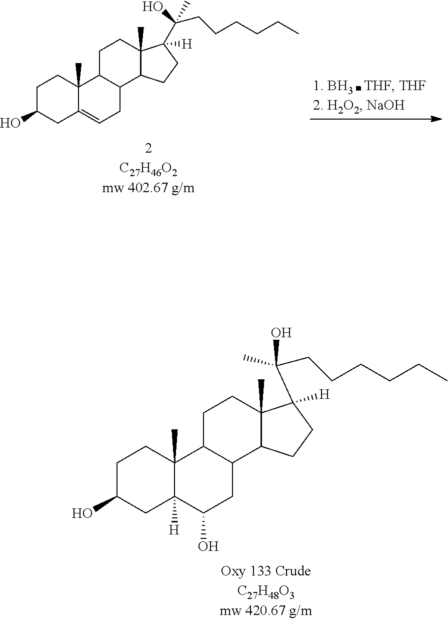 [SMILES: CCCCCC[C@](C)(O)[C@H]1CCC2C3CC=C4C[C@@H](O)CC[C@]4(C)C3CC[C@@]21C ; CCCCCC[C@](C)(O)[C@H]1CCC2C3C[C@H](O)[C@H]4C[C@@H](O)CC[C@]4(C)C3CC[C@@]21C]

The reaction mixture was quenched by addition of a mixture of 10% sodium hydroxide (4750 mL) and 30% hydrogen peroxide (1375 mL). The quench was extremely exothermic and required several hours to complete. The internal temperature was maintained below 10° C. After the addition of the quench volume was complete, the mixture was held cold for 1.5 hours then allowed to warm to room temperature overnight. 8 liters of dichloromethane was then added. The organic layer was isolated and washed with 7 liters of fresh water, and was concentrated under reduced pressure. The product was isolated as a viscous, oily mass which solidified on standing.

The product was dissolved in 4 liters of dichloromethane and was placed onto a silica gel column prepared in dichloromethane. The column was eluted first with 25% ethyl acetate to elute the 7-methyl-7-tridecyl alcohol by-product. Subsequently, the column was eluted with 10% methanol-ethyl acetate to solvate the OXY133. The collected fractions were combined and concentrated under reduced pressure to a waxy solid. The compound was dissolved in acetone-water mixture (3:1) and concentrated under reduced pressure to remove residual solvents. The resulting crude OXY133 was utilized in the next step.

Alternatively, the viscous product recovered from the hydroboration/oxidation can be solidified by stirring with heptanes, and the product isolated by filtration. The isolated product is suspended in methylene chloride (7.3 mL methylene chloride/g solid). The product was isolated by filtration and used as-is in the next step.

### Example 4

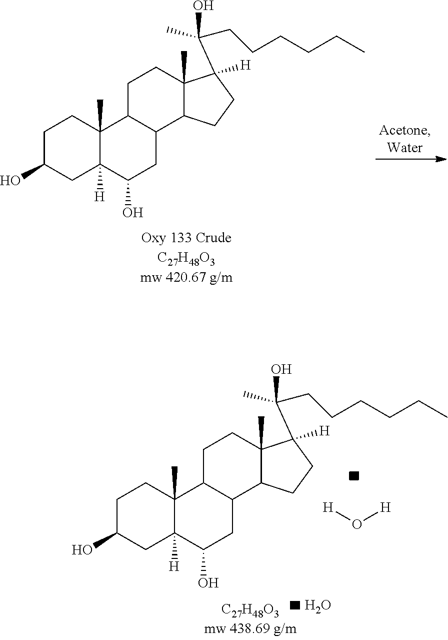

OXY133 was recrystallized by dissolving 630 grams of crude OXY133 into 1500 ml of a 3:1 acetone/water mixture at reflux, then cooling to room temperature. The crystalline solid was recovered by vacuum filtration and dried to afford 336 g, which was a 28% overall yield from compound 1. The OXY133 produced was monohydrous, and has an IUPAC name of (3S,5S,6S,8R,9S,10R,13S,14S,17S)-17-((S)-2-hydroxyoctan-2-yl)-10,13-dimethylhexadecahydro-1H-cyclopenta[a]phenanthrene-3,6-diol, monohydrate.

The ¹H NMR data of OXY133 in CDCl₃ at 400 MHz illustrated the following: δ: 0.66 (m, 1H), 0.85 (m, 10H), 1.23 (m, 18H), 1.47 (m, 9H), 1.68 (m, 4H), 1.81 (m, 1H), 1.99 (m, 1H), 2.06 (m. 1H), 2.18 (m, 1H), 3.42 (m, 1H), 3.58 (m, 1H). The ¹³C NMR data of OXY133 in CDCl₃ at 400 MHz illustrated the following: d: 13.7, 14.0, 14.3, 21.2, 22.5, 22.8, 23.9, 24.4, 26.6, 30.1, 31.1, 32.1, 32.5, 33.9, 36.5, 37.5, 40.4, 41.7, 43.1, 44.3, 51.9, 53.9, 56.5, 57.9, 69.6, 71.3, 75.4. The infrared spectroscopy data of OXY133 showed peaks at 3342 cm⁻¹, 2929 cm⁻¹, 2872 cm⁻¹, 2849 cm⁻¹. The turbo spray mass spectrometry data of the OXY133 showed peaks at 438.4 m/z [M+NH₄]+, 420.4 m/z (M-H₂O+NH₄]+, 403.4 m/z [M-H₂O+H]+, 385.4 m/z [M−2H₂O+H]+.

### Example 5

Alternative One-Vessel Procedure from Pregnenolone Acetate

100 mL n-hexylmagnesium chloride (2M in tetrahydrofuran, 200 mmol) was charged to a flask and cooled to −10° C. A solution containing 20 g pregnenolone acetate (56 mmol) in 200 ml of anhydrous tetrahydrofuran) was added dropwise, while maintaining the internal reaction temperature below −10° C. After the addition was completed, the mixture was stirred for 30 minutes then allowed to warm to room temperature. After 4 hours at room temperature, the mixture had become a gelatinous stirrable mass. The mixture was cooled to 0° C. and 200 mL Borane-tetrahydrofuran complex (1M in tetrahydrofuran, 200 mmol) was added dropwise, while maintaining the internal temperature below 0° C. Once addition was complete, the resulting solution was allowed to warm to room temperature overnight.

The mixture was cooled to 0° C. and quenched by the slow addition of a mixture of 10% NaOH (190 mL) and 30% H₂O₂ (55 mL). Once the quench was complete, the mixture was extracted with MTBE (800 mL total) resulting in an emulsion. Brine was added and the layers were separated. The organic phase was concentrated under reduced pressure to a clear, viscous oil. The oil was further purified utilizing the plug column method previously described.

### Example 6

Preparation of OXY133 Monohydrate from Anhydrous OXY133

2.0 g anhydrous OXY133 was added to 10 mL isopropanol in a 100 mL polyblock reactor. The mixture was heated to 30° C. and then stirred at 30° C. for 45 minutes until the solids were dissolved completely. Acceptable heating temperatures ranged from 25° C. to 35° C. A polish filtration step can be added after dissolving the anhydrous OXY133. The mixture of anhydrous OXY133 and isopropanol was then cooled to 5° C. 20 mL of water was then added to the cooled mixture over 120 minutes at 5° C., resulting in the formation of a precipitate approximately one third of the way through the addition of water. The water addition can also be done in temperature ranges from about 0° C. to about 20° C. with no significant effect on the crystal structure. The resulting mixture of OXY133, isopropanol and water was then stirred at 5° C. for 18 hours. However, it is recommended that the resulting mixture could be mixed for at least 2 hours at 5° C. after the water addition was complete to ensure that all solids were precipitated from the solution. A white solid was collected by rapidly filtering the mixture and then washing the solid with 2.0 mL of an isopropanol:water mixture in a ratio of 1:2 v/v. The filtered and washed solids were then dried in a vacuum oven at 20′C. Acceptable temperatures for drying can range from about 20° C. to about 30′C. It is noted that drying at temperatures above this range resulted in the slow conversion of OXY133 monohydrate to a different, unknown crystal form. The reagents utilized and the properties of the resulting OXY133 monohydrate of OXY133 Form A polymorph are summarized in Table 7 below.

TABLE 7

| Reacted | Molec. Weight | density | | | Lot |
|---|---|---|---|---|---|
| Compound | (g/mol) | (g/mL) | Equivalents | Amount/moles | Number |
| Anhydrous | 420.67 | NA | 1.0 | 2.0 g/.0047 | 55352-23-07 |
| OXY133 | | | | | |
| Isopropanol | NA | 0.786 | NA | 10 mL + 0.67 mL | CML-Bulk |
| Water (H₂O) | NA | 1.00 | NA | 20 mL + 1.33 mL | CML-Bulk |
| Isolated | | | | | Lot |
| Solid | Yield | KF | DSC/TGA | Appearance | Number |
| OXY133 | 1.96 g | 4.07% | 4.76% | white solid | 2891-28-1 |
| Monohydrate | (94% recovery) | | | | |

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A method for preparing an OXY133 polymorph, the method comprising subjecting a slurry of OXY133 to conditions sufficient to convert OXY133 to the OXY133 polymorph, wherein the OXY133 polymorph comprises polymorph Form A, polymorph Form B, polymorph Form C, polymorph Form D, polymorph Form E, polymorph Form F, polymorph Form G, polymorph Form H, polymorph Form I or a mixture thereof.

2. A method of claim 1, wherein the conditions comprise dissolving a slurry of OXY133 in a solvent and precipitating the OXY133 polymorph by adding an anti-solvent at a temperature sufficient to precipitate the OXY133 polymorph, wherein OXY133 comprises anhydrous OXY133 or OXY133 polymorph Form B; an OXY133 polymorph other than polymorph Form B; a hydrate of OXY133; or a solvate of OXY133.

3. A method of claim 2, wherein the conditions to convert to an OXY133 polymorph comprise mixing OXY133 with: (i) an isopropanol solvent, and a water anti-solvent in a ratio from about 1:1 v/v to about 1:2 v/v at a temperature from about 0° C. to about 20° C. to obtain OXY133 polymorph Form A or OXY133 monohydrate; (ii) a tetrahydrofuran solvent, and a water anti-solvent in a ratio of about 1:2 v/v at a temperature from about 10° C. to about 35° C. to obtain OXY133 polymorph Form A or OXY133 monohydrate; (iii) a tetrahydrofuran/acetone solvent, and a water anti-solvent at a temperature of about 35° C. to obtain OXY133 polymorph Form A or OXY133 monohydrate; (iv) an acetone solvent, and a water anti-solvent in a ratio of about 1:1 v/v at a temperature of about 15° C. to about 25° C. to obtain OXY133 polymorph Form A or OXY133 monohydrate; (v) an acetone solvent, and a water anti-solvent in a ratio of about 1:1 v/v at a temperature of about 30° C. to about 60° C. to obtain OXY133 polymorph Form C; (vi) a methanol solvent, and a water anti-solvent in a ratio of about 1:1 v/v at a temperature of about 20° C. to about 70° C. to obtain OXY133 polymorph Form D; (vii) water at a temperature from about 20° C. to about 70° C. to obtain OXY133 polymorph Form E; (viii) an acetone solvent, and a water anti-solvent at a temperature from about 5° C. to about 15° C. to obtain OXY133 polymorph Form F; (ix) an isopropanol solvent, and a water anti-solvent in a ratio of about 1:2 v/v at a temperature of about 40° C. to obtain OXY133 polymorph Form G; (x) an isopropanol solvent, and a water anti-solvent in a ratio of about 1:2 at a temperature of about −10° C. to obtain OXY133 polymorph Form H; (xi) a methanol/acetone solvent, and a water anti-solvent at temperature of about 20° C. to obtain OXY133 polymorph Form I; or (xii) acetone recrystallization at about 20° C. to obtain OXY133 polymorph Form I.

4. A method of claim 1, wherein OXY133 is prepared by reacting a diol having the formula: 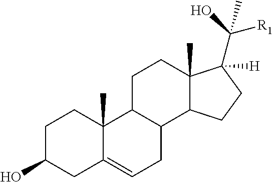
with borane, hydrogen peroxide and tetrahydrofuran to form an oxysterol or a pharmaceutically acceptable salt, hydrate or solvate thereof having the formula: 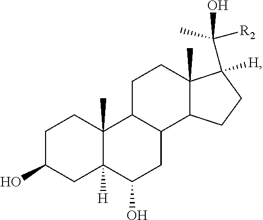
wherein R₁ and R₂ comprise a hexyl group and the diol comprises (3S,8S,9S,10R,13R,14S,17R)-10,13-dimethyl-17-[(S)-2-hydroxyoctan-yl]-2,3,4,7,8,9,11,12,14,15,16,17-dodecahydro-1H-cyclopenta[a]phenanthren-3-ol (OXY133).

5. A method of claim 1, wherein the OXY133 polymorph comprises (i) Form A that produces an X-ray powder diffraction pattern comprising one or more of the following reflections: 16.4, 17.91 and 20.94±0.2 degree 2θ; (ii) Form B that produces an X-ray powder diffraction pattern comprising one or more of the following reflections: 13.3, 16.1, and 18.82±0.2 degree 2θ; or a mixture thereof.

6. A method of claim 5, wherein (i) the X-ray powder diffraction pattern of OXY133 polymorph Form A further comprises one or more of the following reflections: 6.1, 12.3, and 18.6±0.2 degree 2θ; (ii) the X-ray powder diffraction pattern of OXY133 polymorph Form B further comprises one or more of the following reflections: 5.9, 11.9, and 17.96±0.2 degree 2θ; or a mixture thereof.

7. A method of claim 1, wherein a water content of OXY133 monohydrate or OXY133 polymorph Form A comprises a range from about 3.25% to about 4.1% by weight.

8. A method of claim 1, wherein OXY133 monohydrate or OXY133 polymorph Form A has a yield of from about 85% to about 94% by weight.

9. A method of claim 3, further comprising drying OXY133 monohydrate at 20° C.

10. A method of claim 9, wherein the drying occurs in a vacuum or a freeze dryer.

11. A polymorph of OXY133 which comprises polymorph Form A, polymorph Form B, polymorph Form C, polymorph Form D, polymorph Form E, polymorph Form F, polymorph Form G, polymorph Form H, polymorph Form I or a mixture thereof.

12. A polymorph of claim 11, wherein the OXY133 polymorph is polymorph Form A that produces an X-ray powder diffraction pattern comprising one or more of the following reflections: 16.4, 17.9 and 20.9±0.2 degree 2θ.

13. A polymorph of claim 12, wherein the X-ray powder diffraction pattern of polymorph Form A further comprises one or more of the following reflections: 6.1, 12.3, and 18.6±0.2 degree 2θ.

14. A polymorph of claim 11, wherein the OXY133 polymorph is polymorph Form B that produces an X-ray powder diffraction pattern comprising one or more of the following reflections: 13.3, 16.1, and 18.82±0.2 degree 2θ.

15. A polymorph of claim 14, wherein the X-ray powder diffraction pattern of polymorph Form B further comprises one or more of the following reflections: 5.9, 11.9, and 17.96±0.2 degree 2θ.

16. A polymorph of claim 12, wherein polymorph Form A is present in an amount from about 85% to about 94% by weight.

17. A polymorph of claim 12, wherein a water content of polymorph Form A or OXY133 monohydrate comprises from about 3.25% to about 4.1% by weight.

18. A pharmaceutical composition comprising an OXY133 polymorph which comprises polymorph Form A, polymorph Form B, polymorph Form C, polymorph Form D, polymorph Form E, polymorph Form F, polymorph Form G, polymorph Form H, polymorph Form I or a mixture thereof and a pharmaceutically acceptable excipient.

19. A pharmaceutical composition of claim 18, wherein the OXY133 polymorph comprises (i) Form A that produces an X-ray powder diffraction pattern comprising one or more of the following reflections: 16.4, 17.91 and 20.94±0.2 degree 2θ; (ii) Form B that produces an X-ray powder diffraction pattern comprising one or more of the following reflections: 13.3, 16.1, and 18.82±0.2 degree 2θ; or a mixture thereof.

20. A pharmaceutical composition of claim 19, wherein (i) the X-ray powder diffraction pattern of OXY133 polymorph Form A further comprises one or more of the following reflections: 6.1, 12.3, and 18.6±0.2 degree 2θ; (ii) the X-ray powder diffraction pattern of OXY133 polymorph Form B further comprises one or more of the following reflections: 5.9, 11.9, and 17.96±0.2 degree 2θ; or a mixture thereof.
